(19) United States
(12) Patent Application Publication
(10) Pub. No.: US 2020/0157190 A1
(43) Pub. Date: May 21, 2020

(54) MONOVALENT AND DIVALENT BINDING PROTEINS

(71) Applicant: ABCAM PLC, Cambridge (GB)

(72) Inventors: Jacinto VILLANUEVA, San Ramon, CA (US); Reem MAHRAT, Pleasanton, CA (US)

(21) Appl. No.: 16/470,533

(22) PCT Filed: Dec. 18, 2017
(86) PCT No.: PCT/US2017/067029

§ 371 Date: Jun. 17, 2019

Related U.S. Application Data
(60) Provisional application No. 62/436,214, filed on Dec. 19, 2016.

(51) Int. Cl. C07K 16/00; G01N 33/53

(52) U.S. Cl. CPC C07K 16/00; G01N 33/53; C07K 2317/526; C07K 2317/522; C07K 2317/35

(57) ABSTRACT

Provided herein are engineered monovalent and divalent antibodies having high specificity and affinity, as well as the use of such antibodies as research, diagnostic and therapeutic agents.

### CROSS REFERENCE TO RELATED APPLICATIONS

This application is a 35 U.S.C. 371 national stage filing of International Application No. PCT/US2017/067029, filed Dec. 18, 2017, which claims priority from U.S. Provisional Patent Application No. 62/436,214, filed Dec. 19, 2016. The contents of the aforementioned applications are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Sep. 20, 2019, is named CBJ-002US SL.txt and is 168,015 bytes in size.

### BACKGROUND

Engineered proteins, such as multispecific antibodies capable of binding two or more antigens, are known in the art. Specifically, efforts to increase the valency or the number of antigenic determinants that an individual antibody molecule can bind have lead to the development of bi-specific antibodies (see, for example, Jimenez et al., Molecular Cancer Therapeutics 2005:4(3)427-434, Lu et al. J. of Immun. Methods 1999:230, 159-171 and U.S. Patent Publication Nos. 20070014794 and 20050100543), as well as alternative antibody formats (see, for example, WO 2013/003652, U.S. Pat. No. 7,612,181, US2002/0127231, and US2002/0004587).

Such multispecific binding proteins can be generated using cell fusion, chemical conjugation, or recombinant DNA techniques. In particular, to improve efficiency of production of multiple epitope binding antibodies, a variety of recombinant methods have been developed. For example, methods of efficient production of bispecific antbodies have been developed, both as antibody fragments (Carter et al. (1995); Pluckthun et al (1997) Immunotechology 3:83-105; Todorovska et al. (2001) J. Immunol. Methods 248:47-66) and full length IgG formats (Carter (2001) J. Immunol. Methods 248:7-15). For example, production of homogeneous full-length IgG-like bispecific antbodies has been achieved by the so-called “knobs-into-holes” engineering for efficient Ig CH3 domain heterodimerization (Ridgway et al. 1996 Protein Eng. 9:617-21; Merchant et al. 1998 Nat. Biotech. 16:677-81) and by fusing single chain Fvs (scFv) of different specificities onto either the N- or the C-terminus of a full-length IgG molecule (Zhuang et al. Protein Eng. 2000 13:361-7; Coloma and Morrison Nat. Biotechnol. 199715:159-63). BsAbs have also been constructed by genetically fusing two single chain Fv (scFv) or Fab fragments with or without the use of flexible linkers (Mallender et al. J. Biol. Chem. 1994 269:199-206; Mack et al. Proc. Natl. Acad. Sci. USA. 1995 92:7021-5; Zapata et al. Protein Eng. 1995 8.1057-62), via a dimerization device such as leucine zipper (Kostelny et al. J. Immunol. 1992148:1 547-53; de Kruif et al. J. Biol. Chem. 1996 271:7630-4) and Ig C.lamda./CH1 domains (Muller et al. FEBS Lett. 422:259-64); by diabody (Holliger et al. (1993) Proc. Nat. Acad. Sci. USA. 1998 90:6444-8; Zhu et al. Bio/Technology (NY) 1996 14:192-6); Fab-scFv fusion (Schoonjans et al. J. Immunol. 2000 165:7050-7); and mini-antibody formats (Pack et al. Biochemistry 1992 31:1579-84; Pack et al. Bio/Technology 1993 11:1271-7).

Despite the advantages associated with alternative antibody formats, there is a need in the art for additional and improved functional binding proteins capable of binding epitopes with high specificity and affinity.

### SUMMARY

Provided herein are engineered monovalent and divalent antibodies having high specificity and affinity, as well as the use of such antibodies as diagnostic, therapeutic, and research agents.

In one aspect, a monovalent heavy chain binding protein is provided, comprising in amino to carboxyl terminal order, (a) a first heavy chain variable domain and all or a portion of a CH1 domain linked to (b) a second heavy chain variable domain and all or a portion of a constant region. In one embodiment, the monovalent heavy chain binding protein further comprises a full-length constant region comprising a hinge region, a CH2 domain, and a CH3 domain.

In another embodiment, the monovalent heavy chain binding protein further comprises a tag. Suitable tags include, but are not limited, to CBP, FLAG, GST, HA, HBH, MBP, Myc, polyhistidine, S-tag, SUMO, TAP, TRX, and V5. In one embodiment, the tag is linked to the carboxyl terminus of the CH3 domain. In another embodiment, the tag replaces the hinge to the CH3 domain(s).

In another embodiment, the first and second variable domains of the monovalent heavy chain bind to the same epitope. In another embodiment, the first and second variable domains comprise identical amino acid sequences. In another embodiment, the monovalent heavy chain binding protein is as depicted in FIG. 1. In another document, the first and second variable domains bind to two different epitopes. In another embodiment, the first and second variable domains comprise different amino acid sequences. In another embodiment, the monovalent heavy chain binding protein is as depicted in FIG. 2.

In another embodiment, the first variable domain and CH1 domain are linked to the second variable domain by a linker comprising a two amino acid repeat of proline (P) and Glutamine (Q) (e.g., comprising 3, 4, 5, 6, 7, 8, 9, 10, 11, 12, 13, 15, 15, 16, 17, 18, 19, 20, 21, 22, 23, 24, or 25 pairs of proline (P) and Glutamine (Q)). In another embodiment, the linker comprises Asparagine (N), Serine (S), Glycine (G), and/or Threonine (T). In another embodiment, the linker comprises the amino acid sequence set forth in SEQ ID NO:10. In another embodiment, the linker consists of the amino acid sequence set forth in SEQ ID NO:10. Glutamine (Q)].

The first and/or second variable domains can comprise any suitable amino acid sequence. In one embodiment, the first and/or second variable domains of the monovalent heavy chain binding protein comprise an amino acid sequence selected from the group consisting of SEQ ID NO:34 (corresponding to the heavy chain variable region of Antibody 1), SEQ ID NO:35 (corresponding to the heavy chain variable region of Antibody 2), and SEQ ID NO:36 (corresponding to the heavy chain variable region of Antibody 3). In a further embodiment, the light chain comprises the amino acid sequence set forth in SEQ ID NO:38 (corresponding to the light chain of Construct #1) and/or SEQ ID NO:40 (corresponding to the light chain of Construct #2).

In another embodiment, the monovalent heavy chain binding protein comprises an amino acid sequence selected from the group consisting of SEQ ID NO:21 (corresponding to Heavy Chain Construct #1), SEQ ID NO:24 (corresponding to Heavy Chain Construct #2), SEQ ID NO:27 (corresponding to Heavy Chain Construct #3), SEQ ID NO:30 (corresponding to Heavy Chain Construct #4), and SEQ ID NO:33 (corresponding to Heavy Chain Construct #5).

In another embodiment, the constant region of the monovalent heavy chain binding protein is an IgG1, IgG2, IgG3, IgG4, IgM, IgA1, IgA2, IgAsec, IgD, or IgE isotype.

In another embodiment, the monovalent heavy chain binding protein is a two chain monovalent heavy chain binding protein, wherein the first and second heavy chain variable domains are each paired (e.g., associated) with a light chain, e.g., via a disulfide bond. In one embodiment, the light chains of the two chain monovalent heavy chain binding protein are not linked to one another.

In another embodiment, the first and second variable domains of the two chain monovalent heavy chain bind to the same epitope. In another embodiment, the first and second variable domains comprise identical amino acid sequences. In another embodiment, the two chain monovalent heavy chain binding protein is as depicted in FIG. 3. In another document, the first and second variable domains bind to two different epitopes. In another embodiment, the first and second variable domains comprise different amino acid sequences. In another embodiment, the two chain monovalent heavy chain binding protein is as depicted in FIG. 4.

In another aspect, a single chain divalent heavy chain binding protein is provided, wherein the single chain divalent heavy chain binding protein comprises two of the single chain monovalent heavy chain binding proteins described herein. In one embodiment, the two monovalent heavy chain binding proteins of the single chain divalent heavy chain binding protein are linked by a disulfide bond. In another embodiment, the first and second variable domains of each of the monovalent heavy chains bind to the same epitope. In another embodiment, the first and second variable domains of each of the monovalent heavy chains comprise identical amino acid sequences. In another embodiment, the single chain divalent heavy chain binding protein is as depicted in FIG. 5. In another document, the first and second variable domains of each of the monovalent heavy chains bind to two different epitopes. In another embodiment, the first and second variable domains of each of the monovalent heavy chains comprise different amino acid sequences. In another embodiment, the single chain divalent heavy chain binding protein is as depicted in FIG. 6.

In another aspect, a two chain divalent heavy chain binding protein is provided, wherein the two chain divalent heavy chain binding protein comprises two of the two chain monovalent heavy chain binding proteins described herein. In one embodiment, the two monovalent two heavy chain binding proteins are linked by a disulfide bond. In another embodiment, the variable domains bind to the same epitope. In another embodiment, the variable domains comprise identical amino acid sequences. In another embodiment, the two chain divalent heavy chain binding protein is as depicted in FIG. 7. In another document, the first and second variable domains of each of the monovalent heavy chains bind to two different epitopes. In another embodiment, the first and second variable domains of each of the monovalent heavy chains comprise different amino acid sequences. In another embodiment, the two chain divalent heavy chain binding protein is as depicted in FIG. 8.

The variable domains of the monovalent or divalent heavy chain binding proteins described herein can be derived from any suitable source. In one embodiment, the variable domains are derived from a human, rabbit, mouse, rat, shark, human, goat, chicken, llama, or other camelid species. In another embodiment, the variable domains are derived from a monoclonal antibody.

The monovalent and divalent heavy chain binding proteins described herein are engineered to have increased binding specificity and affinity. In one embodiment, the binding affinity of the single chain monovalent heavy chain binding protein is synergistically increased compared to the binding affinity of the same binding protein having only a single variable domain. In another embodiment, the binding affinity of the two chain monovalent heavy chain binding protein is synergistically increased compared to the binding affinity of the same binding protein having only a single heavy variable domain and a single light chain variable domain. In another embodiment, the binding affinity of the single chain divalent heavy chain binding protein is synergistically increased compared to the binding affinity of the same binding protein wherein each chain has only one variable domain. In another embodiment, the binding affinity of the two chain divalent heavy chain binding protein is synergistically increased compared to the binding affinity of the same binding protein wherein each chain has only one variable domain.

In another embodiment, the increase in binding is subpicomolar. In another embodiment, the synergistic increase is about a 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90, fold, 95-fold, 100-fold, 105-fold, 110-fold, 115-fold, 120-fold, 125-fold, 130-fold, 135-fold, 140-fold, 145-fold, 150-fold, 155-fold, 160-fold, 165-fold, 170-fold, 175-fold, 180-fold, 185-fold, 190-fold, 195-fold, 200-fold, 205-fold, 210-fold, 215-fold, 220-fold, 225-fold, 230-fold, 235-fold, 240-fold, 245-fold, 250-fold, 255-fold, 260-fold, 265-fold, 270-fold, 275-fold, 280-fold, 285-fold, 290-fold, 295-fold, 300-fold, 305-fold, 310-fold, 315-fold, 320-fold, 325-fold, 330-fold, 335-fold, 340-fold, 345-fold, 350-fold, 355-fold, 360-fold, 365-fold, 370-fold, 375-fold, 380-fold, 385-fold, 390-fold, 395-fold, 400-fold, 405-fold, 410-fold, 415-fold, 420-fold, 425-fold, 430-fold, 435-fold, 440-fold, 445-fold, 450-fold, 455-fold, 460-fold, 465-fold, 470-fold, 475-fold, 480-fold, 485-fold, 490-fold, 495-fold, 500-fold, 505-fold, 510-fold, 515-fold, 520-fold, 525-fold, 530-fold, 535-fold, 540-fold, 545-fold, 550-fold, 555-fold, 560-fold, 565-fold, 570-fold, 575-fold, 580-fold, 585-fold, 590-fold, 595-fold, or 600-fold increase. In one embodiment, the synergistic increase is a 20-fold increase. In another embodiment, the synergistic increase is a 454-fold increase.

The monovalent and divalent binding proteins described herein are capable of binding one or more target molecules of interest, including, but not limited to: ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL 22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1(E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21 Wap1/Cip1); CDKN1B (p27Kip1); CDKN1C; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); C-MET; CMKLR1; CMKOR1 (RDC1); CNR1; COL18A1; COL1A1; COL4A3; COL6A1; CR2; CRP; CSF1(M-CSF); CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10 (IP-10); CXCL11(I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; DAB21P; DES; DKFZp451J0118; DNCL1; DPP4; E2F1; ECGF1; EDG1; EFNA1; EGFR, EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); FADD; FasL; FASN; FCER1A; FCER2; FCGR3A; FGF; FGF1 (aFGF); FGF10; FGF1; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSL1 (FRA-1); FY (DARC); GABRP (GABAa); GAGEB1; GAGEC1; GALNAC4S-6ST; GATA3; GDF5; GFI1; GGT1; GITR; GM-CSF; GNAS1; GNRH1; GPR2 (CCR10); GPR31; GPR44; GPR81 (FKSG80); GRCC10 (C10); GRP; GSN (Gelsolin); GSTP1; HAVCR2; HDAC4; HDAC5; HDAC7A; HDAC9; HER; HGF; HIF1A; HIP1; histamine and histamine receptors; HLA-A; HLA-DRA; HM74; HMOX1; HUMCYT2A; ICEBERG; ICOSL; ID2; IFN-α; IFNA1; IFNA2; IFNA4; IFNA5; IFNA6; IFNA7; IFNB1; IFNgamma; IFNW1; IGBP1; IGF; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2 IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMAS; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NROB1; NROB2; NR1D1; NR1D2; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGF; PDGFA; PDGFB; PECAM1; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p21Rac2); RARB; RGS1; RGS13; RGS3; RNF110 (ZNF144); ROBO2; S100A2; SCGB1D2 (lipophilin B); SCGB2A1(mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Sprl); ST6GAL1; STAB1; STATE; STEAP; STEAP2; TB4R2; TBX21; TCP10; TDGF1; TEK; TGFA; TGFB1; TGFB1I1; TGFB2; TGFB3; TGFB1; TGFBR1; TGFBR2; TGFBR3; TH1L; THB S1 (thrombospondin-1); THBS2; THBS4; THPO; TIE (Tie-1); TIGIT; TIMP3; tissue factor; TLR10; TLR2; TLR3; TLR4; TLR5; TLR6; TLR7; TLR8; TLR9; TNF; TNF-α; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNF SF13 (April); TNFSF13B; TNF SF14 (HVEM-L); TNF SF15 (VEGI); TNFSF18; TNFSF4 (0X40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase Iia); TP53; TPM1; TPM2; TRADD; TRAF1; TRAF2; TRAF3; TRAF4; TRAF5; TRAF6; TREM1; TREM2; TRPC6; TSLP; TWEAK; VEGF; VEGFB; VEGFC; versican; VHL C5; VLA-4; XCL1 (lymphotactin); XCL2 (SCM-1b); XCR1 (GPR5/CCXCR1); YY1; and ZFPM2. In one embodiment, the target molecule is present on the surface or within a cell. In another embodiment, the target molecule is secreted outside of a cell.

Compositions comprising the monovalent or divalent heavy chain binding proteins described herein and a carrier are also provided, as well as kits comprising the monovalent or divalent heavy chain binding proteins described herein and instructions for use.

Further provided are methods of detecting the presence or absence of a target molecule of interest in a biological sample comprising (A) contacting a sample with any of the monovalent or divalent heavy chain binding proteins described herein, wherein the binding protein specifically binds the target molecule of interest, and (B) detecting the presence or absence of at least one complex comprising the binding protein and target molecule of interest. In one embodiment, the biological sample is from a human patient.

Also provided are methods of diagnosing a patient as having a disease characterized by a target molecule of interest, comprising (A) contacting a sample obtained from the patient with any of the monovalent or divalent heavy chain binding proteins described herein, wherein the binding protein binds the target molecule of interest, and (B) diagnosing the patient as having the disease based on detection of at least one complex comprising the binding protein and target molecule of interest.

The target molecule of interest for use in the methods of detection and methods of diagnosis as described herein include any suitable molecule of interest, including those described herein the section entitled “Target Molecules”.

Methods of treating a patient (e.g., a human patient) having a disease (e.g., a cancer, infectious disease, inflammatory or autoimmune disorder) by administering an effective amount of any of the monovalent or divalent heavy chain binding proteins described herein, thereby treating the disease. In one embodiment, the method comprises administering an additional therapeutic agent to the patient. In a particular embodiment, the disease is a cancer selected from the group consisting of melanoma (e.g., metastatic malignant melanoma), renal cancer (e.g. clear cell carcinoma), prostate cancer (e.g. hormone refractory prostate adenocarcinoma), pancreatic adenocarcinoma, breast cancer, colon cancer, lung cancer (e.g. non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In another embodiment, the disease is an inflammatory or autoimmune disorder is selected from the group consisting of Crohn's disease, ulcerative colitis, inflammatory bowel disease, inflammatory fibrosis, scleroderma, lung fibrosis, and cirrhosis, rheumatoid arthritis (RA), osteoarthritis, osteoporosis, asthma (including allergic asthma), allergies, chronic obstructive pulmonary disease (COPD), multiple sclerosis, psoriasis, uveitis, graft versus host disease (GVHD), juvenile early-onset Type I diabetes, transplant rejection, SLE, and Sjögren's syndrome. In another embodiment, the disease is an infectious disease selected from the group consisting of human immunodeficiency viruses, hepatitis viruses class A, B and C, Eppstein Barr virus, human cytomegalovirus, human papilloma viruses, and herpes viruses.

Further provided are methods of increasing (e.g., synergistically increasing) the binding affinity of a binding protein (e.g., a monovalent or divalent binding protein) by linking one or more additional heavy chain variable regions to the amino terminus of the binding protein. In one embodiment, the additional heavy chain variable region is the same as (e.g., has the same amino acid sequence) the heavy chain variable region of the binding protein (e.g., VR1 and VR1). In another embodiment, the additional heavy chain variable region binds to the same epitope as the heavy chain variable region of the binding protein (e.g., VR1 and VR2). In another embodiment, the additional heavy chain variable region binds to the same target molecule as the heavy chain variable region of the binding protein (e.g., VR1 and VR2).

In one embodiment, the binding protein is a single chain binding protein. In another embodiment, the binding protein is a monovalent binding protein. In another embodiment, the binding protein is a single chain monovalent binding protein. In another embodiment, the binding protein is a two chain binding protein. In another embodiment, the binding protein is a two chain monovalent binding protein. In another embodiment, the binding protein is a divalent binding protein. In another embodiment, the binding protein is a two chain divalent binding protein. In another embodiment, the binding increases to subpicomolar levels.

Also provided are nucleic acids encoding the monovalent or divalent heavy chain binding proteins described herein, as well as expression vectors comprising such nucleic acids and host cells. In another aspect, methods of producing the monovalent or divalent binding proteins described herein comprising: culturing a host cell in culture medium under conditions wherein the nucleic acid sequence is expressed, thereby producing the binding protein; and recovering the binding protein from the host cell or culture medium. In another embodiment, the monovalent or divalent binding protein is coexpressed with a light chain.

### BRIEF DESCRIPTION OF THE DRAWINGS

FIG. 1 depicts an exemplary structure of a monovalent single chain (heavy) variable domain binding protein that recognizes one epitope (BD1 and BD1).

FIG. 2 depicts an exemplary structure of a monovalent single chain (heavy) variable domain binding protein that recognizes two different epitopes (BD1 and BD2).

FIG. 3 depicts an exemplary structure of a monovalent two chain (heavy and light) variable domain binding protein that recognizes one epitope (BD1 and BD1).

FIG. 4 depicts an exemplary structure of a monovalent two chain (heavy and light) variable domain binding protein that recognizes two different epitopes (BD1 and BD2).

FIG. 5 depicts an exemplary structure of a divalent single chain (heavy) variable domain binding protein that recognizes two different epitopes (BD1, BD2, BD1, and BD2).

FIG. 6 depicts an exemplary structure of a divalent single chain (heavy) variable domain binding protein that recognizes one epitope (BD1, BD1, BD1, and BD1).

FIG. 7 depicts an exemplary structure of a divalent two chain (heavy and light) variable domain binding protein that recognizes one epitope (BD1, BD1, BD1, and BD1).

FIG. 8 depicts an exemplary structure of a divalent two chain (heavy and light) variable domain binding protein that recognizes two different epitopes (BD1, BD2, BD1, and BD2).

FIG. 9 is a schematic depicting the heavy and light chain constructs.

FIG. 10 depicts the results of ELISA testing for specificity with two-chain secreted antibodies.

FIG. 11 is a graph which illustrates the sensitivity and specificity of the two-chain secreted antibodies by ELISA.

FIG. 12 depicts the IHC results of a stacked rabbit two-chain monoclonal antibody against Ag1 and Ag2.

FIG. 13 is a graph which illustrates the sensitivity and specificity of the single-chain secreted antibodies by ELISA. The stacked sample (Ab1-Ab1) and the original rabbit monoclonal (Ab1) was tested against Ag1 (positive control antigen) and Ag2 (negative control antigen) for specificity.

### DETAILED DESCRIPTION

Provided herein are engineered monovalent and divalent antibodies having high specificity and affinity, as well as the use of such antibodies as research, diagnostic and therapeutic agents.

### Definitions

For convenience, the meaning of certain terms and phrases used in the specification, examples, and appended claims, are provided below.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

One of ordinary skill in the art will appreciate that starting materials, biological and chemical materials, biological and chemical reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this disclosure.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although aspects of the present invention have been specifically disclosed by various embodiments which may include preferred embodiments, exemplary embodiments and optional features, modifications and variations of the concepts herein disclosed may be resorted to by those skilled in the art. Such modifications and variations are considered to be within the scope of embodiments of the invention as described and as may be defined by the appended claims.

The term “polypeptide” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “antibody” as used to herein includes whole antibodies and any antigen binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. In certain naturally occurring antibodies, the heavy chain constant region is comprised of three domains, CHL CH2 and CH3. In certain naturally occurring antibodies, each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 5×10⁻⁹ M or less, and most preferably between 10⁻⁷ M and 10⁻¹⁰ M or less, but does not bind with high affinity to unrelated antigens.

An immunoglobulin may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. In certain embodiments, the antibodies described herein are of the IgG1 or IgG2 subtype. Immunoglobulins, e.g., IgG1, exist in several allotypes, which differ from each other in at most a few amino acids. “Antibody” includes, by way of example, both naturally occurring and non-naturally occurring antibodies; monoclonal and polyclonal antibodies; chimeric and humanized antibodies; human and nonhuman antibodies; wholly synthetic antibodies; and single chain antibodies.

“Antigen binding site” refers to a binding site that comprises the VH and/or VL domain of an antibody, or at least one CDR thereof, provided that the antigen binding site binds specifically to its target antigen. For example, an antigen binding site may comprise, consist essentially of, or consist of a VHCDR3 alone or together with a VHCDR2 and optionally a VHCDR1. In certain embodiments, an antigen binding site comprises a VH domain and a VL domain, which may be present on the same polypeptide or on two different polypeptides, e.g., the VH domain is present on a heavy chain and a VL domain is present on a light chain.

“Antigen-binding portion” of an antibody refers to one or more fragments of an antibody that retain the ability to specifically bind to an antigen. It has been shown that the antigen-binding function of an antibody can be retained by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment which consists of a VH domain; and (vi) an isolated Complementarity Determining Region (“CDR”). Furthermore, although VL and VH are two domains of an Fv fragment, VL and VH are coded for by separate genes, they can be joined, using recombinant methods, by a synthetic linker that enables them to be made as a single protein chain in which the VL and VH regions pair to form monovalent proteins, known as single chain Fvs (scFvs) (see, e.g., U.S. Pat. No. 5,892,019). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from ITGB4 are tested for reactivity with the given anti-ITGBR antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)). The term “epitope mapping” refers to the process of identification of the molecular determinants for antibody-antigen recognition.

“Binding affinity” refers to the strength of a binding interaction and includes both the actual binding affinity as well as the apparent binding affinity. The actual binding affinity is a ratio of the association rate over the disassociation rate. The apparent affinity can include, for example, the avidity resulting from a polyvalent interaction. Dissociation constant (Kd), is typically the reciprocal of the binding affinity, and may be conveniently measured using a surface plasmon resonance assay (e.g., as determined using a ForteBio Octet platform (Pall ForteBio Corp.), a BIACORE 3000 instrument (GE Healthcare) or a cell binding assay, examples of which assays are described in Example 3 of U.S. Pat. No. 7,846,440.

“Specific binding,” “specifically binds,” “selective binding,” and “selectively binds,” as well as “binds specifically” “binds selectively,” when referring to the binding of a binding site to its target epitope or a combination of binding sites to their target epitopes, means that the binding site(s) exhibit(s) immunospecific binding to the target epitope(s). A binding site that binds specifically to an epitope exhibits appreciable affinity for a target epitope and, generally, does not exhibit cross-reactivity with other epitopes in that it does not exhibit appreciable affinity to any unrelated epitope and preferably does not exhibit affinity for any unrelated epitope that is equal to, greater than, or within two orders of magnitude lower than the affinity for the target epitope. “Appreciable” or preferred binding includes binding with a dissociation constant (Kd) of 10⁻⁸, 10⁻⁹M, 10⁻¹⁰, 10⁻¹¹, 10⁻¹² M, 10⁻¹³ M or an even lower Kd value. The Kd values can also be indicated as 10e-8, 10e-9 M, etc. Note that lower values for Kd (dissociation constant) indicate higher binding affinity, thus a Kd of 10⁻⁷ is a higher Kd value than a Kd of 10⁻⁸, but indicates a lower binding affinity than a Kd of 10⁻⁸. Dissociation constants with values of about 10⁻⁷ M, and even as low as about 10⁻⁸M, are at the high end of dissociation constants suitable for therapeutic antibodies. Binding affinities may be indicated by a range of dissociation constants, for example, 10⁻⁷ to 10⁻¹²M, 10⁻⁷ to 10⁻¹²M, 10⁻⁸ to 10⁻¹²M or better (i.e., or lower value dissociation constant). Dissociation constants in the nanomolar (10⁻⁹M) to picomolar (10⁻¹²M) range or lower are typically most useful for therapeutic antibodies. Suitable dissociation constants are Kds of 50 nM or less (i.e., a binding affinity of 50 nM or higher—e.g., a Kd of 45 nM) or Kds of 40 nM, 30 nM, 20 nM, 10 nM, 1 nM, 100 pM, 10 pM or 1 pM or less. Specific or selective binding can be determined according to any art-recognized means for determining such binding, including, for example, according to Scatchard analysis, competitive binding assays, ELISA, flow cytometry, fluorescence microscopy, Western blotting). Methods for analyzing binding affinity, cross-reactivity, and binding kinetics include standard assays known in the art, for example, Biacore™ surface plasmon resonance (SPR) analysis using a Biacore™ 2000 SPR instrument (Biacore AB, Uppsala, Sweden).

“CDR” or “complementarity determining region” refers to the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other.

“CH1 domain” refers to the heavy chain immunoglobulin constant domain located between the VH domain and the hinge. It spans EU positions 118-215. A CH1 domain may be a naturally occurring CH1 domain, or a naturally occurring CH1 domain in which one or more amino acids have been substituted, added or deleted, provided that the CH1 domain has the desired biological properties. A desired biological activity may be a natural biological activity, an enhanced biological activity or a reduced biological activity relative to the naturally occurring sequence.

“CH2 domain” refers to the heavy chain immunoglobulin constant domain that is located between the hinge and the CH3 domain. As defined here, it spans EU positions 237-340. A CH2 domain may be a naturally occurring CH2 domain, or a naturally occurring CH2 domain in which one or more amino acids have been substituted, added or deleted, provided that the CH2 domain has the desired biological properties. A desired biological activity may be a natural biological activity, an enhanced biological activity or a reduced biological activity relative to that of the naturally occurring domain.

“CH3 domain” refers to the heavy chain immunoglobulin constant domain that is located C-terminally of the CH2 domain and spans approximately 110 residues from the N-terminus of the CH2 domain, e.g., about positions 341-446b (EU numbering system). A CH3 domain may be a naturally occurring CH3 domain, or a naturally occurring CH3 domain in which one or more amino acids have been substituted, added or deleted, provided that the CH3 domain has the desired biological properties. A desired biological activity may be a natural biological activity, an enhanced biological activity or a reduced biological activity relative to that of the naturally occurring domain. A CH3 domain may or may not comprise a C-terminal lysine.

“CH4 domain” refers to the heavy chain immunoglobulin constant domain that is located C-terminally of the CH3 domain in IgM and IgE antibodies. A CH4 domain may be a naturally occurring CH4 domain, or a naturally occurring CH4 domain in which one or more amino acids have been substituted, added or deleted, provided that the CH4 domain has the desired biological properties. A desired biological activity may be a natural biological activity, an enhanced biological activity or a reduced biological activity relative to that of the naturally occurring domain.

“CL domain” refers to the light chain immunoglobulin constant domain that is located C-terminally to the VL domain. It spans about Kabat positions 107A-216. A CL domain may be a naturally occurring CL domain, or a naturally occurring CL domain in which one or more amino acids have been substituted, added or deleted, provided that the CL domain has the desired biological properties. A desired biological activity may be a natural biological activity, an enhanced biological activity or a reduced biological activity relative to that of the naturally occurring domain. A CL domain may or may not comprise a C-terminal lysine.

A “constant region” or domain of a light chain of an immunoglobulin is referred to interchangeably as a “CL,” “light chain constant region domain,” “CL region” or “CL domain.” A “constant region” or domain on a heavy chain (e.g., hinge, CH1, CH2 or CH3 domains) of an immunoglobulin is referred to interchangeably as a “CH,” “heavy chain constant domain,” “CH” region or “CH domain.”

A variable domain on an immunoglobulin light chain is referred to interchangeably as a “VL,” “light chain variable domain,” “VL region” or “VL domain.”

A variable domain on an immunoglobulin heavy chain is referred to interchangeably as a “VH,” “heavy chain variable domain,” “VH region” or “VH domain.”

“Domain” refers generally to a region, e.g., an independently folding, globular region or a non-globular region (e.g., a linker domain), of a heavy or light chain polypeptide which may comprise peptide loops (e.g., 1 to 4 peptide loops) that may be stabilized, for example, by a f3-pleated sheet and/or an intrachain disulfide bond. The constant and variable regions of immunoglobulin heavy and light chains are typically folded into domains. In particular, each one of the CH1, CH2, CH3, CH4, CL, VH and VL domains typically form a loop structure. “EU” indicates that amino acid positions in a heavy chain constant region, including amino acid positions in the CH1, hinge, CH2, and CH3 domains, are numbered herein according to the EU index numbering system (see Kabat et al., in “Sequences of Proteins of Immunological Interest”, U.S. Dept. Health and Human Services, 5^{th} edition, 1991).

“Fab” refers to the antigen binding portion of an antibody, comprising two chains: a first chain that comprises a VH domain and a CH1 domain and a second chain that comprises a VL domain and a CL domain. Although a Fab is typically described as the N-terminal fragment of an antibody that was treated with papain and comprises a portion of the hinge region, it is also used herein as referring to a binding domain wherein the heavy chain does not comprise a portion of the hinge.

“Fc region” refers to the portion of a single immunoglobulin heavy chain beginning in the hinge region just upstream of the papain cleavage site (i.e. residue 216 in IgG, taking the first residue of heavy chain constant region to be 114) and ending at the C-terminus of the antibody. Accordingly, a complete Fc region comprises at least a hinge, a CH2 domain, and a CH3 domain. Two Fc regions that are dimerized are referred to as “Fc” or “Fc dimer.” An Fc region may be a naturally occurring Fc region, or a naturally occurring Fc region in which one or more amino acids have been substituted, added or deleted, provided that the Fc region has the desired biological properties. A desired biological activity may be a natural biological activity, an enhanced biological activity or a reduced biological activity relative to that of the naturally occurring domain.

“Framework region” or “FR” or “FR region” includes the amino acid residues that are part of the variable region, but are not part of the CDRs (e.g., using the Kabat definition of CDRs). Therefore, a variable region framework is between about 100-120 amino acids in length but includes only those amino acids outside of the CDRs. For the specific example of a heavy chain variable region and for the CDRs as defined by Kabat et al., 1991, ibid., framework region 1 corresponds to the domain of the variable region encompassing amino acids 1-30; framework region 2 corresponds to the domain of the variable region encompassing amino acids 36-49; framework region 3 corresponds to the domain of the variable region encompassing amino acids 66-94, and framework region 4 corresponds to the domain of the variable region from amino acids 103 to the end of the variable region. The framework regions for the light chain are similarly separated by each of the light chain variable region CDRs. Similarly, using the definition of CDRs by Chothia et al. or McCallum et al. the framework region boundaries are separated by the respective CDR termini as described above. In preferred embodiments, the CDRs are as defined by Kabat.

“Full length antibody” or “full length Ab” is an antibody (“Ab”) that comprises one or more heavy chains and one or more light chains, which optionally may be connected. Each heavy chain is comprised of a heavy chain variable region (abbreviated herein as VH) and a heavy chain constant region. The heavy chain constant region is comprised of three domains CH1, CH2, and CH3, and optionally a fourth domain, CH4. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is typically composed of three CDRs and four FRs, arranged from amino-terminus to carboxyl-terminus in the following order: FR1, CDR1, FR2, CDR2, FR3, CDR3, and FR4. Immunoglobulin proteins can be of any type or class (e.g., IgG, IgE, IgM, IgD, IgA and IgY) or subclass (e.g., IgG1, IgG2, IgG3, IgG4, IgA1 and IgA2).

“Hinge” or “hinge region” or “hinge domain” refers to the flexible portion of a heavy chain located between the CH1 domain and the CH2 domain. It is approximately 25 amino acids long, and is divided into an “upper hinge,” a “middle hinge” or “core hinge,” and a “lower hinge.” A hinge may be a naturally occurring hinge, or a naturally occurring hinge in which one or more amino acids have been substituted, added or deleted, provided that the hinge has the desired biological properties. A desired biological activity may be a natural biological activity, an enhanced biological activity or a reduced biological activity relative to the naturally occurring sequence.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, monoclonal antibodies are advantageous in that they can be synthesized by hybridoma cells that are uncontaminated by other immunoglobulin producing cells. Alternative production methods are known to those trained in the art, for example, a monoclonal antibody may be produced by cells stably or transiently transfected with the heavy and light chain genes encoding the monoclonal antibody.

The modifier “monoclonal” indicates the character of the antibody as being obtained from a substantially homogeneous population of antibodies, and is not to be construed as requiring engineering of the antibody by any particular method. The term “monoclonal” is used herein to refer to an antibody that is derived from a clonal population of cells, including any eukaryotic, prokaryotic, or phage clone, and not the method by which the antibody was engineered. For example, the monoclonal antibodies to be used in accordance with the present invention may be made by the hybridoma method first described by Kohler et al., Nature, 256:495 (1975), or may be made by any recombinant DNA method (see, e.g., U.S. Pat. No. 4,816,567), including isolation from phage antibody libraries using the techniques described in Clackson et al., Nature, 352:624-628 (1991) and Marks et al., J. Mol. Biol., 222:581-597 (1991), for example. These methods can be used to produce monoclonal mammalian, chimeric, humanized, human, domain antibodies, single chain diabodies, vaccibodies, and linear antibodies.

The term “chimeric” antibodies includes antibodies in which at least one portion of the heavy and/or light chain is identical with or homologous to corresponding sequences in antibodies derived from a particular species or belonging to a particular antibody class or subclass, and at least one other portion of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)).

“Humanized” forms of nonhuman (e.g., murine) antibodies are chimeric antibodies that contain minimal sequence derived from nonhuman immunoglobulin. For the most part, humanized antibodies are human immunoglobulins (recipient antibody) in which the native CDR residues are replaced by residues from the corresponding CDR of a nonhuman species (donor antibody) such as mouse, rat, rabbit or nonhuman primate having the desired specificity, affinity, and capacity. In some instances, FW region residues of the human immunoglobulin are replaced by corresponding nonhuman residues. Furthermore, humanized antibodies may comprise residues that are not found in the recipient antibody or in the donor antibody. These modifications are made to further refine antibody performance. In general, a humanized antibody heavy or light chain will comprise substantially all of at least one or more variable domains, in which all or substantially all of the CDRs correspond to those of a nonhuman immunoglobulin and all or substantially all of the FWs are those of a human immunoglobulin sequence. In certain embodiments, the humanized antibody will comprise at least a portion of an immunoglobulin constant region (Fc), typically that of a human immunoglobulin. For further details, see, Jones et al., Nature, 321:522-525 (1986); Riechmann et al., Nature, 332:323-329 (1988); and Presta, Curr. Op. Struct. Biol., 2:593-596 (1992).

A “human antibody” can be an antibody derived from a human or an antibody obtained from a transgenic organism that has been “engineered” to produce specific human antibodies in response to antigenic challenge and can be produced by any method known in the art. In certain techniques, elements of the human heavy and light chain loci are introduced into strains of the organism derived from embryonic stem cell lines that contain targeted disruptions of the endogenous heavy chain and light chain loci. The transgenic organism can synthesize human antibodies specific for human antigens, and the organism can be used to produce human antibody-secreting hybridomas. A human antibody can also be an antibody wherein the heavy and light chains are encoded by a nucleotide sequence derived from one or more sources of human DNA. A fully human antibody also can be constructed by genetic or chromosomal transfection methods, as well as phage display technology, or in vitro activated B cells, all of which are known in the art.

“% identical” refers to two or more nucleic acid or polypeptide sequences or subsequences that are the same (100% identical) or have a specified percentage of nucleotide or amino acid residues that are the same, when the two sequences are aligned for maximum correspondence and compared. To align for maximum correspondence, gaps may be introduced into one of the sequences being compared. The amino acid residues or nucleotides at corresponding positions are then compared and quantified. When a position in the first sequence is occupied by the same residue as the corresponding position in the second sequence, then the sequences are identical at that position. The percent identity between the two sequences is a function of the number of identical positions shared by the sequences (e.g., % identity=# of identical positions/total # of positions (e.g., overlapping positions)×100). In certain embodiments, the two sequences are the same length. The determination that one sequence is a measured % identical with another sequence can be determined using a mathematical algorithm. A non-limiting example of a mathematical algorithm utilized for such comparison of two sequences is incorporated in the ALIGN program (version 2.0) which is part of the GCG sequence alignment software package. When utilizing the ALIGN program e.g., for comparing amino acid sequences, a PAM120 weight residue table, a gap length penalty of 12, and a gap penalty of 4 may be used. Additional algorithms for sequence analysis are well known in the art and many are available online.

The term “polynucleotide(s)” refers to nucleic acids such as DNA molecules and RNA molecules and analogs thereof (e.g., DNA or RNA generated using nucleotide analogs or using nucleic acid chemistry). As desired, the polynucleotides may be made synthetically, e.g., using art-recognized nucleic acid chemistry or enzymatically using, e.g., a polymerase, and, if desired, be modified. Typical modifications include methylation, biotinylation, and other art-known modifications. In addition, the nucleic acid molecule can be single-stranded or double-stranded and, where desired, linked to a detectable moiety.

The term “variant” with respect to a reference polypeptide refers to a polypeptide that possesses at least one amino acid mutation or modification (i.e., alteration) as compared to a native polypeptide. Variants generated by “amino acid modifications” can be produced, for example, by substituting, deleting, inserting and/or chemically modifying at least one amino acid in the native amino acid sequence.

An “amino acid modification” refers to a change in the amino acid sequence of a predetermined amino acid sequence. Exemplary modifications include an amino acid substitution, insertion and/or deletion.

An “amino acid modification at” a specified position, refers to the substitution or deletion of the specified residue, or the insertion of at least one amino acid residue adjacent the specified residue. By insertion “adjacent” a specified residue is meant insertion within one to two residues thereof. The insertion may be N-terminal or C-terminal to the specified residue.

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (Gin); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). Substitution with one or more non-naturally occurring amino acid residues is also encompassed by the definition of an amino acid substitution herein.

A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301 336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA.

An “amino acid insertion” refers to the incorporation of at least one amino acid into a predetermined amino acid sequence. While the insertion will usually consist of the insertion of one or two amino acid residues, the present application contemplates larger “peptide insertions”, e.g. insertion of about three to about five or even up to about ten amino acid residues. The inserted residue(s) may be naturally occurring or non-naturally occurring as disclosed above.

An “amino acid deletion” refers to the removal of at least one amino acid residue from a predetermined amino acid sequence.

The term “mutagenesis” refers to, unless otherwise specified, any art recognized technique for altering a polynucleotide or polypeptide sequence. Preferred types of mutagenesis include error prone PCR mutagenesis, saturation mutagenesis, or other site directed mutagenesis.

“Site-directed mutagenesis” is a technique standard in the art, and is conducted using a synthetic oligonucleotide primer complementary to a single-stranded phage DNA to be mutagenized except for limited mismatching, representing the desired mutation. Briefly, the synthetic oligonucleotide is used as a primer to direct synthesis of a strand complementary to the single-stranded phage DNA, and the resulting double-stranded DNA is transformed into a phage-supporting host bacterium. Cultures of the transformed bacteria are plated in top agar, permitting plaque formation from single cells that harbor the phage. Theoretically, 50% of the new plaques will contain the phage having, as a single strand, the mutated form; 50% will have the original sequence. Plaques of interest are selected by hybridizing with kinased synthetic primer at a temperature that permits hybridization of an exact match, but at which the mismatches with the original strand are sufficient to prevent hybridization. Plaques that hybridize with the probe are then selected, sequenced and cultured, and the DNA is recovered.

A “phage display library” is a protein expression library that expresses a collection of cloned protein sequences as fusions with a phage coat protein. Thus, the phrase “phage display library” refers herein to a collection of phage (e.g., filamentous phage) wherein the phage express an external (typically heterologous) protein. The external protein is free to interact with (bind to) other moieties with which the phage are contacted. Each phage displaying an external protein is a “member” of the phage display library.

The term “panning” is used to refer to the multiple rounds of screening process in identification and isolation of phages carrying compounds, such as antibodies, with high affinity and specificity to a target.

1. Single Chain Monovalent Binding Proteins

A single chain monovalent heavy chain binding protein refers to a construct comprising in amino to carboxyl terminal order, (a) a first heavy chain variable domain and all or a portion of a CH1 domain linked to (b) a second heavy chain variable domain and all or a portion of a constant region. In one embodiment, the monovalent heavy chain binding protein further comprises a full-length constant region comprising a hinge region, a CH2 domain, and a CH3 domain.

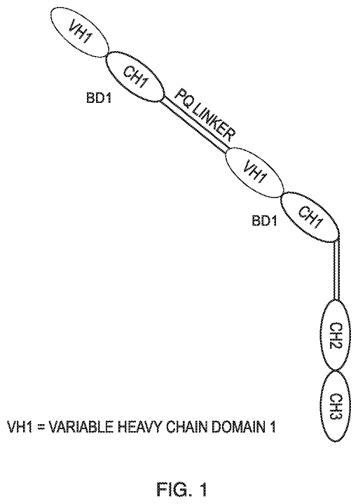

In one embodiment, the first and second variable domains of the monovalent heavy chain bind to the same epitope. In another embodiment, the first and second variable domains comprise identical amino acid sequences. In another embodiment, the monovalent heavy chain binding protein is as depicted in FIG. 1.

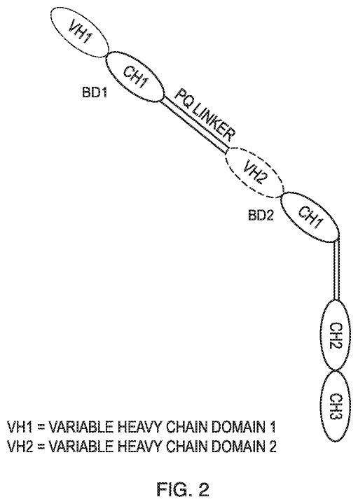

In another document, the first and second variable domains bind to two different epitopes. In another embodiment, the first and second variable domains comprise different amino acid sequences. In another embodiment, the monovalent heavy chain binding protein is as depicted in FIG. 2.

2. Two Chain Monovalent Binding Proteins

A two chain monovalent heavy chain binding protein refers to a monovalent heavy chain binding protein, wherein the first and second heavy chain variable domains are each paired (e.g., associated) with a light chain, e.g., via a disulfide bond.

In one embodiment, the light chains of the two chain monovalent heavy chain binding protein are not linked to one another.

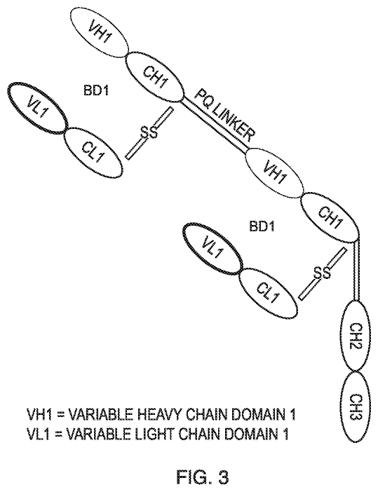

In another embodiment, the first and second variable domains of the two chain monovalent heavy chain bind to the same epitope. In another embodiment, the first and second variable domains comprise identical amino acid sequences. In another embodiment, the two chain monovalent heavy chain binding protein is as depicted in FIG. 3.

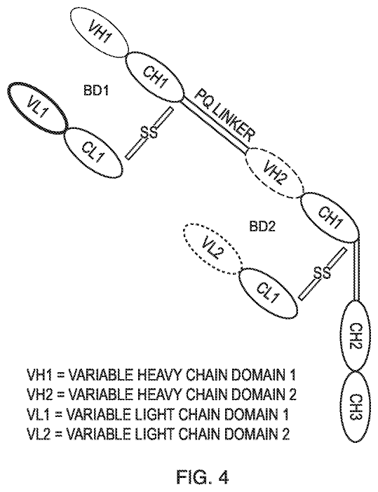

In another embodiment, the first and second variable domains bind to two different epitopes. In another embodiment, the first and second variable domains comprise different amino acid sequences. In another embodiment, the two chain monovalent heavy chain binding protein is as depicted in FIG. 4.

3. Single Chain Divalent Binding Proteins

A single chain divalent heavy chain binding protein refers to a construct comprising two of the single chain monovalent heavy chain binding proteins described herein. In one embodiment, the two monovalent heavy chain binding proteins of the single chain divalent heavy chain binding protein are linked by a disulfide bond.

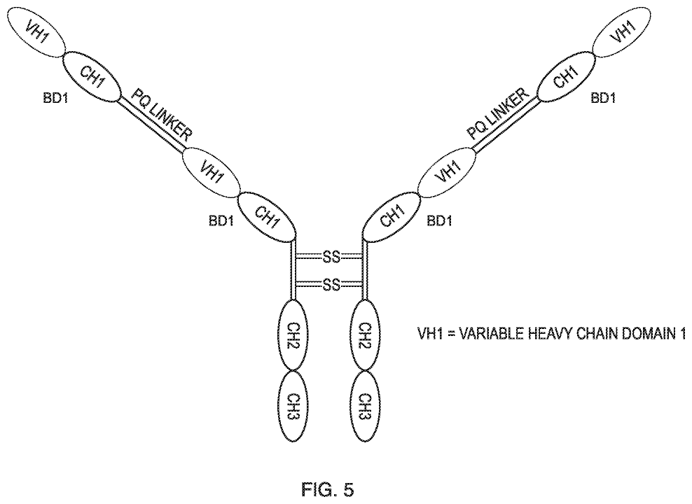

In one embodiment, the first and second variable domains of each of the monovalent heavy chains bind to the same epitope. In another embodiment, the first and second variable domains of each of the monovalent heavy chains comprise identical amino acid sequences. In another embodiment, the single chain divalent heavy chain binding protein is as depicted in FIG. 5.

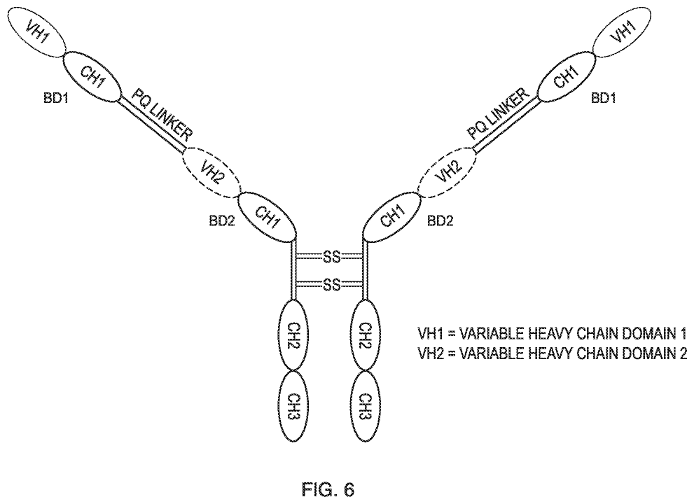

In another document, the first and second variable domains of each of the monovalent heavy chains bind to two different epitopes. In another embodiment, the first and second variable domains of each of the monovalent heavy chains comprise different amino acid sequences. In another embodiment, the single chain divalent heavy chain binding protein is as depicted in FIG. 6.

4. Divalent Two Chain Binding Proteins

A divalent two chain heavy chain binding protein refers to a construct comprising two of the two chain monovalent heavy chain binding proteins described herein. In one embodiment, the two monovalent two heavy chain binding proteins are linked by a disulfide bond.

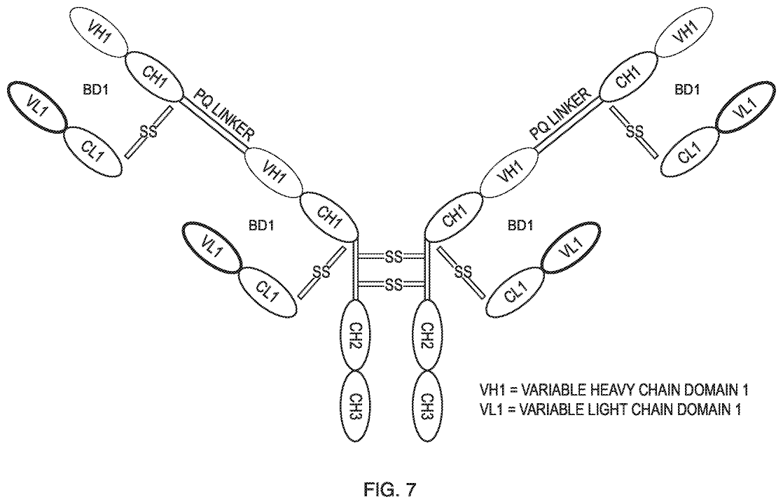

In one embodiment, the variable domains bind to the same epitope. In another embodiment, the variable domains comprise identical amino acid sequences. In another embodiment, the two chain divalent heavy chain binding protein is as depicted in FIG. 7.

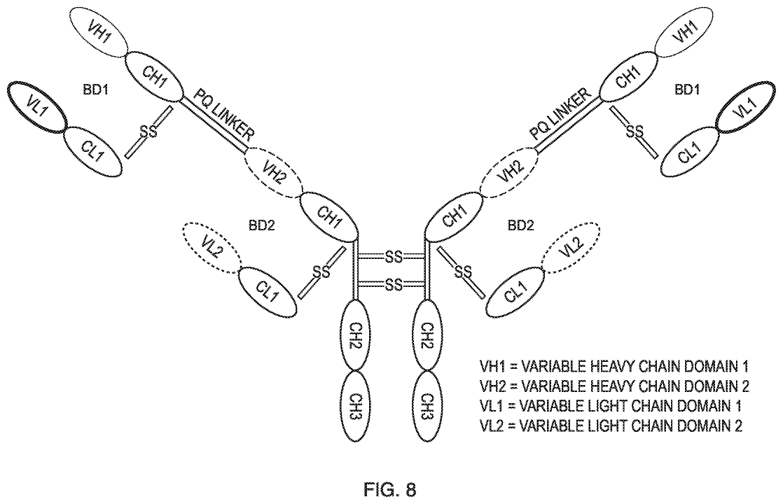

In another document, the first and second variable domains of each of the monovalent heavy chains bind to two different epitopes. In another embodiment, the first and second variable domains of each of the monovalent heavy chains comprise different amino acid sequences. In another embodiment, the two chain divalent heavy chain binding protein is as depicted in FIG. 8.

5. Exemplary Sequences

The sequences of the monovalent or divalent heavy chain binding proteins described herein can be derived from any suitable source. In one embodiment, the variable domains are derived from a human, rabbit, mouse, rat, shark, human, goat, chicken, llama, or other camelid species. In another embodiment, the variable domains are derived from a monoclonal antibody.

In one embodiment, the signal peptide used is one that is found naturally upstream from the 2-chain or single-chain antibody and contains the N-terminal methionine. For rabbit antibodies, the typical leader sequence and corresponding nucleotide sequence can consist of:

(SEQ ID NO: 1)
atggagactgggctgcgctggcttctcctggtcgctgtgctcaaaggtg
tccagtgt
(SEQ ID NO: 2)
M E T G L R W L L L V A V L K G V Q C

For llama single chain antibodies, the leader sequence and corresponding nucleotide sequence can consist of:

(SEQ ID NO: 3)
atggagaggggctgagagggtggtcctggctgactactacaaggtgtc
caggctgag
(SEQ ID NO: 4)
M E L G L S W V V L A A L L Q G V Q A E

In one embodiment, the variable domain and CH1 domain of the first heavy chain consists of all CDRs and framework regions between the CDRs, as well as the complete CH1 domain. This region is in-frame with the signal peptide. For rabbit antibodies, this can contain the cysteine residue [C] of the heavy chain that is connected with a cysteine residue of the light chain, e.g., as set forth below.

(SEQ ID NO: 5)
cagtcgctggaggagtccgggggtcgcctggtcacgcctgggacacccctgacactcacc
(SEQ ID NO: 6)
Q S L E E S G G R L V T P G T P L T L T
tgcacagtctctggaatcgacctcacttcctactacatgacctgggtccgccagggtcca
C T V S G I D L T S Y Y M T W V R Q G P
gggaaggggctggaatggatcggcatcattagtactgatggtagcgcatcctacgcgaac
G K G L E W I G I I S T D G S A S Y A N
tgggcgaaaggccgattcaccatctccaaaacctcgaccacggtggatctgaaaatcacc
W A K G R F T I S K T S T T V D L K I T
agtccgacaaccgacgacacggccacctatttttgtgccagagcttacgatacttatggt
S P T T D D T A T Y F C A R A Y D T Y G
tcgggttatgatggtgtctttggcatctggggcccaggcaccctggtcaccgtctcctca
S G Y D G V F G I W G P G T L V T V S S
gggcaacctaaggctccatcagtatcccactggccccctgctgcggggacacacccagc
G Q P K A P S V F P L A P C C G D T P S
tccacggtgaccctgggctgcctggtcaaaggctacctcccggagccagtgaccgtgacc
S T V T L G C L V K G Y L P E P V T V T
tggaactcgggcaccctcaccaatggggtacgcaccttcccgtccgtccggcagtcctca
W N S G T L T N G V R T F P S V R Q S S
ggcctctactcgctgagcagcgtggtgagcgtgacctcaagcagccagcccgtcacctgc
G L Y S L S S V V S V T S S S Q P V T C
aacgtggcccacccagccaccaacaccaaagtggacaagaccgttgcgccctcgacatgc
N V A H P A T N T K V D K T V A P S T C
agcaagcccacgtgcccaccccctgaactcctg
S K P T C P P P E L L

For llama antibodies, the HHV1 and CH1 domains of the first antibody can consist of:

(SEQ ID NO: 7)
gtgcagaggtggagtagggggaggcttggtgcggcctggggggtactgagactcatc
(SEQ ID NO: 8)
V Q L V E S G G G L V R P G G S L R L I
tgtgaagcctttggaagcatcttcagcgtgaataccatgggctggtaccgccagaccccg
C E A F G S I F S V N T M G W Y R Q T P
gggaagcagcgggagttggtcgcaattatgactagtgaaggtacgacaaattatgcagat
G K Q R E L V A I M T S E G T T N Y A D
ttcgtgaagggccgattcaccatctccagagacaacgccaaggacacagtgtatctacaa
F V K G R F T I S R D N A K D T V Y L Q
atgaacagcctgaattctgaggacacggccgtctattattgtctcttgaaattgccattt
M N S L N S E D T A V Y Y C L L K L P F
acccttaaggagtattggggccaggggacccaggtcaccgtctccgcagaacccaagaca
T L K E Y W G Q G T Q V T V S A E P K T
ccaaaa
P K

In one embodiment, the variable domain and CH1 domain, hinge region, and CH2 and CH3 domains of the second heavy chain exclude any natural signal peptide and is in-frame with the PQ Linker. Essentially, this includes all of the sequence found between but not including the signal peptide and the region downstream from the translational stop site. For 2-chain rabbit antibodies, the sequence can consist of:

(SEQ ID NO: 11)
cagtcgctggaggagtccgggggtcgcctggtcacgcctgggacacccctgacactcacc
(SEQ ID NO: 12)
Q S L E E S G G R L V T P G T P L T L T
tgcacagtctctggaatcgacctcacttcctactacatgacctgggtccgccagggtcca
C T V S G I D L T S Y Y M T W V R Q G P
gggaaggggctggaatggatcggcatcattagtactgatggtagcgcatcctacgcgaac
G K G L E W I G I I S T D G S A S Y A N
tgggcgaaaggccgattcaccatctccaaaacctcgaccacggtggatctgaaaatcacc
W A K G R F T I S K T S T T V D L K I T
agtccgacaaccgacgacacggccacctattrngtgccagagcttacgatacttatggt
S P T T D D T A T Y F C A R A Y D T Y G
tcgggttatgatggtgtctttggcatctggggcccaggcaccctggtcaccgtctcctca
S G Y D G V F G I W G P G T L V T V S S
gggcaacctaaggctccatcagtcttcccactggccccctgctgcggggacacacccagc
G Q P K A P S V F P L A P C C G D T P S
tccacggtgaccctgggctgcctggtcaaaggctacctcccggagccagtgaccgtgacc
S T V T L G C L V K G Y L P E P V T V T
tggaactcgggcaccctcaccaatggggtacgcaccttcccgtccgtccggcagtcctca
W N S G T L T N G V R T F P S V R Q S S
ggcctctactcgctgagcagcgtggtgagcgtgacctcaagcagccagcccgtcacctgc
G L Y S L S S V V S V T S S S Q P V T C
aacgtggcccacccagccaccaacaccaaagtggacaagaccgttgcgccctcgacatgc
N V A H P A T N T K V D K T V A P S T C
agcaagcccacgtgcccaccccctgaactcctggggggaccgtctgtcttcatcttcccc
S K P T C P P P E L L G G P S V F I F P
ccaaaacccaaggacaccctcatgatctcacgcacccccgaggtcacatgcgtggtggtg
P K P K D T L M I S R T P E V T C V V V
gacgtgagccaggatgaccccgaggtgcagttcacatggtacataaacaacgagcaggtg
D V S Q D D P E V Q F T W Y I N N E Q V
cgcaccgcccggccgccgctacgggagcagcagttcaacagcacgatccgcgtggtcagc
R T A R P P L R E Q Q F N S T I R V V S
accctccccatcgcgcaccaggactggctgaggggcaaggagttcaagtgcaaagtccac
T L P I A H Q D W L R G K E F K C K V H
aacaaggcactcccggcccccatcgagaaaaccatctccaaagccagagggcagcccctg
N K A L P A P I E K T I S K A R G Q P L
gagccgaaggtctacaccatgggccctccccgggaggagctgagcagcaggtcggtcagc
E P K V Y T M G P P R E E L S S R S V S
ctggcctgcatgatcaacggcttctacccttccgacatctcggtggagtgggagaagaac
L A C M I N G F Y P S D I S V E W E K N
gggaaggcagaggacaactacaagaccacgccggccgtgctggacagcgacggctcctac
G K A E D N Y K T T P A V L D S D G S Y
ttcctctacagcaagctctcagtgcccacgagtgagtggcagcggggcgacgtcttcacc
F L Y S K L S V P T S E W Q R G D V F T
tgctccgtgatgcacgaggccttgcacaaccactacacgcagaagtccatctcccgctct
C S V M H E A L H N H Y T Q K S I S R S
ccgggtaaatga
P G K -

For single-chain llama antibodies, the sequence can consist of:

(SEQ ID NO: 13)
gctcaggtgcagctggtggagtctgggggaggcttggtgcagcctggggggtctctgagg
(SEQ ID NO: 14)
A Q V Q L V E S G G G L V Q P G G S L R
ctctcctgtgtagcctctggaaacatgttcggcattaacacgatgggctggttccgcgcg
L S C V A S G N M F G I N T M G W F R A
gctccagggaaagagcgtcagtttgtagcagctgataatttcagtggtggcaggaagaat
A P G K E R Q F V A A D N F S G G R K N
attgacttctccgtgaggggtcgattcaccatgtcaagagacgccgccagcaacacgata
I D F S V R G R F T M S R D A A S N T I
tatctacaaatgaacagcctgaaagttgaggacacggccgtttattactgcgcagcgggt
Y L Q M N S L K V E D T A V Y Y C A A G
gcgaatacgatgatgattgtggcggacaattacgaatactggggtcaggggacccaggtc
A N T M M I V A D N Y E Y W G Q G T Q V
accgtctcctcagaacccaagacaccaaaaaagcttccacagcctcagccgcagccccag
T V S S E P K T P K K L P Q P Q P Q P Q
ccacaaccacaacccaatcctacaacagaatccaagtgtcccaaatgtccagcccctgag
P Q P Q P N P T T E S K C P K C P A P E
ctcctgggagggccctcagtcttcatcttccccccgaaacccaaggacgtcctctccatt
L L G G P S V F I F P P K P K D V L S I
tctgggaggcccgaggtcacgtgcgttgtggtagacgtgggccaggaagaccccgaggtc
S G R P E V T C V V V D V G Q E D P E V
agtttcaactggtacattgatggcgctgaggtgcgaacggccaacacgaggccaaaagag
S F N W Y I D G A E V R T A N T R P K E
gaacagttcaacagcacgtaccgcgtggtcagcgtcctgcccatccagcaccaggactgg
E Q F N S T Y R V V S V L P I Q H Q D W
ctgacggggaaggaattcaagtgcaaggtcaacaacaaagctctcccggcccccatcgag
L T G K E F K C K V N N K A L P A P I E
aagaccatctccaaggccaaagggcagacccgggagccgcaggtgtacgccctggcccca
K T I S K A K G Q T R E P Q V Y A L A P
caccgggaagagctggccaaggacaccgtgagcgtaacctgcctggtcaaaggcttctac
H R E E L A K D T V S V T C L V K G F Y
ccacctgatatcaacgttgagtggcagaggaacggtcagccggagtcagagggcacctac
P P D I N V E W Q R N G Q P E S E G T Y
gccaccacgccaccccagctggacaacgacgggacctacttcctctacagcaagctctcg
A T T P P Q L D N D G T Y F L Y S K L S
gtgggaaagaacacgtggcagcggggagaaaccttcacctgtgtggtgatgcatgaggcc
V G K N T W Q R G E T F T C V V M H E A
ctgtga
L -

The first and/or second variable domains can comprise any suitable amino acid sequence.

In one embodiment, the first and/or second variable domains of the monovalent heavy chain binding protein comprise an amino acid sequence selected from the group consisting of SEQ ID NO:34 (corresponding to the heavy chain variable region of Antibody 1), SEQ ID NO:35 (corresponding to the heavy chain variable region of Antibody 2), and SEQ ID NO:36 (corresponding to the heavy chain variable region of Antibody 3).

In a further embodiment, the light chain comprises the amino acid sequence set forth in SEQ ID NO:38 (corresponding to the light chain of Construct #1) and/or SEQ ID NO:40 (corresponding to the light chain of Construct #2).

In another embodiment, the monovalent heavy chain binding protein comprises an amino acid sequence selected from the group consisting of SEQ ID NO:21 (corresponding to Heavy Chain Construct #1), SEQ ID NO:24 (corresponding to Heavy Chain Construct #2), SEQ ID NO:27 (corresponding to Heavy Chain Construct #3), SEQ ID NO:30 (corresponding to Heavy Chain Construct #4), and SEQ ID NO:33 (corresponding to Heavy Chain Construct #5).

6. Linkers

A variety of linkers can be used in the binding proteins described herein. “Linked to” refers to direct or indirect linkage or connection of, in context, amino acids or nucleotides. “Linker” refers to one or more amino acids connecting two domains or regions together. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). Additional linkers suitable for use can be found in the Registry of Standard Biological Parts at http://partsregistry.org/Protein_domains/Linker (see also, e.g., Crasto CJ and Feng JA. LINKER: a program to generate linker sequences for fusion proteins. Protein Eng 2000 May; 13(5) 309-12 and George RA and Heringa J. An analysis of protein domain linkers: their classification and role in protein folding. Protein Eng 2002 November; 15(11) 871-9). A linker may be 1-10, 10-20, 20-30, 30-40, 40-50, 50-60, 60-70, 70-80, 80-90 or at least 90-100 amino acids long.

In one embodiment, the first variable domain and CH1 domain of the monovalent binding proteins described herein are linked to the second variable domain by a linker comprising a two amino acid repeat of proline (P) and Glutamine (Q) (e.g., comprising 3, 4, 5, 6, 7, 8, 9, 10, 11, 12, 13, 15, 15, 16, 17, 18, 19, 20, 21, 22, 23, 24, or 25 pairs of proline (P) and Glutamine (Q)). In another embodiment, the linker comprises Asparagine (N), Serine (S), and/or Threonine (T).

In another embodiment, the linker is a 2-amino acid repeat of 16 pairs of Proline (P) and Glutamine (Q) that is in-frame with the variable domain and CH1 domain of the first heavy chain and comprises the following sequences:

(SEQ ID NO: 9)
cctcaaccccaaccacaaccgcaacctcagccccagccacagccgcagc
cgcagccacaaccccagcctcaaccacaaccccagcctcaacctcag
(SEQ ID NO: 10)
P Q P Q P Q P Q P Q P Q P Q P Q P Q P Q P Q P Q P
Q P Q P Q P Q

In one embodiment, the linker comprises the amino acid sequence set forth in SEQ ID NO:10. In another embodiment, the linker consists of the amino acid sequence set forth in SEQ ID NO:10. Glutamine (Q)].

In another embodiment, the linker is not a CH1, CH2, CH3, or FC domain.

7. Tags

The binding proteins described herein can further comprise a tag. Suitable tags include, but are not limited, to CBP, G-ST, HA, Mitt MBP, Myc, polyhistidine, S-tag, SUMO, TAP, TRX, and V5. In one embodiment, the tag is linked to the carboxyl terminus of the CH3 domain. In another embodiment, the tag replaces the hinge to the CH3 domain(s).

An exemplary construct containing variations of the C-terminus (and corresponding 3′ DNA sequences) of the second single heavy chain are set forth below.

a) His-tag fused to the end of the CH3 domain:

(SEQ ID NO: 15)
gctcaggtgcagctggtggagtctgggggaggcttggtgcagcctggggggtctctgagg
(SEQ ID NO: 16)
A Q V Q L V E S G G G L V Q P G G S L R
ctctcctgtgtagcctctggaaacatgttcggcattaacacgatgggctggttccgcgcg
L S C V A S G N M F G I N T M G W F R A
gctccagggaaagagcgtcagtttgtagcagctgataatttcagtggtggcaggaagaat
A P G K E R Q F V A A D N F S G G R K N
attgacttctccgtgaggggtcgattcaccatgtcaagagacgccgccagcaacacgata
I D F S V R G R F T M S R D A A S N T I
tatctacaaatgaacagcctgaaagttgaggacacggccgtttattactgcgcagcgggt
Y L Q M N S L K V E D T A V Y Y C A A G
gcgaatacgatgatgattgtggcggacaattacgaatactggggtcaggggacccaggtc
A N T M M I V A D N Y E Y W G Q G T Q V
accgtctcctcagaacccaagacaccaaaaaagcttccacagcctcagccgcagccccag
T V S S E P K T P K K L P Q P Q P Q P Q
ccacaaccacaacccaatcctacaacagaatccaagtgtcccaaatgtccagcccctgag
P Q P Q P N P T T E S K C P K C P A P E
ctcctgggagggccctcagtcttcatcttccccccgaaacccaaggacgtcctctccatt
L L G G P S V F I F P P K P K D V L S I
tctgggaggcccgaggtcacgtgcgttgtggtagacgtgggccaggaagaccccgaggtc
S G R P E V T C V V V D V G Q E D P E V
agtttcaactggtacattgatggcgctgaggtgcgaacggccaacacgaggccaaaagag
S F N W Y I D G A E V R T A N T R P K E
gaacagttcaacagcacgtaccgcgtggtcagcgtcctgcccatccagcaccaggactgg
E Q F N S T Y R V V S V L P I Q H Q D W
ctgacggggaaggaattcaagtgcaaggtcaacaacaaagctctcccggcccccatcgag
L T G K E F K C K V N N K A L P A P I E
aagaccatctccaaggccaaagggcagacccgggagccgcaggtgtacgccctggcccca
K T I S K A K G Q T R E P Q V Y A L A P
caccgggaagagctggccaaggacaccgtgagcgtaacctgcctggtcaaaggcttctac
H R E E L A K D T V S V T C L V K G F Y
ccacctgatatcaacgttgagtggcagaggaacggtcagccggagtcagagggcacctac
P P D I N V E W Q R N G Q P E S E G T Y
gccaccacgccaccccagctggacaacgacgggacctacttcctctacagcaagctctcg
A T T P P Q L D N D G T Y F L Y S K L S
gtgggaaagaacacgtggcagcggggagaaaccttcacctgtgtggtgatgcatgaggcc
V G K N T W Q R G E T F T C V V M H E A
ctggttccctctagagggcccttcgaaggtaagcctatccctaaccctctcctcggtctc
L V P S R G P F E G K P I P N P L L G L
gattctacgcgtaccggtcatcatcaccatcaccatcaccatggaggacagtga
D S T R T G H H H H H H H H G G Q -

b) His-tag fused to CH1 domain:

(SEQ ID NO: 17)
gtgcagctggtggagtctgggggaggcttggtgcagcctggggggtctctgaggctccc
(SEQ ID NO: 18)
V Q L V E S G G G L V Q P G G S L R L S
tgtgtagcctctggaaacatgttcggcattaacacgatgggctggttccgcgcggctcca
C V A S G N M F G I N T M G W F R A A P
gggaaagagcgtcagtttgtagcagctgataatttcagtggtggcaggaagaatattgac
G K E R Q F V A A D N F S G G R K N I D
ttctccgtgaggggtcgattcaccatgtcaagagacgccgccagcaacacgatatatcta
F S V R G R F T M S R D A A S N T I Y L
caaatgaacagcctgaaagttgaggacacggccgtttattactgcgcagcgggtgcgaat
Q M N S L K V E D T A V Y Y C A A G A N
acgatgatgattgtggcggacaattacgaatactggggtcaggggacccaggtcaccgtc
T M M I V A D N Y E Y W G Q G T Q V T V
tcctcagaacccaagacaccaaaaagcttgatatcacgcgtaccggtcatcatcaccat
S S E P K T P K K L D I T R T G H H H H
caccatcaccatggaggacagtgaagatcccccgacctcgacctctggctaataaaggaa
H H H H G G Q -

8. Target Molecules

The monovalent and divalent binding proteins described herein are capable of binding one or more target molecules of interest, including, but not limited to: ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; ANGPT1; ANGPT2; ANGPTL3; ANGPTL4; ANPEP; APC; APOC1; AR; AZGP1 (zinc-a-glycoprotein); B7.1; B7.2; BAD; BAFF; BAG1; BAI1; BCL2; BCL6; BDNF; BLNK; BLR1 (MDR15); BlyS; BMP1; BMP2; BMP3B (GDF10); BMP4; BMP6; BMP8; BMPR1A; BMPR1B; BMPR2; BPAG1 (plectin); BRCA1; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP-1); MCAF; CCL20 (MIP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3/CMKBR3); CCR4; CCR5 (CMKBR5/ChemR13); CCR6 (CMKBR6/CKR-L3/STRL 22/DRY6); CCR7 (CKR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD-22; CD24; CD28; CD3; CD37; CD38; CD3E; CD3G; CD3Z; CD4; CD40; CD40L; CD44; CD45RB; CD52; CD69; CD72; CD74; CD79A; CD79B; CD8; CD80; CD81; CD83; CD86; CDH1(E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21 Wap1/Cip1); CDKN1B (p27Kip1); CDKN1C; CDKN2A (p16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3; CLDN7 (claudin-7); CLN3; CLU (clusterin); C-MET; CMKLR1; CMKOR1 (RDC1); CNR1; COL18A1; COL1A1; COL4A3; COL6A1; CR2; CRP; CSF1(M-CSF); CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10 (IP-10); CXCL11(I-TAC/IP-9); CXCL12 (SDF1); CXCL13; CXCL14; CXCL16; CXCL2 (GRO2); CXCL3 (GRO3); CXCL5 (ENA-78/LIX); CXCL6 (GCP-2); CXCL9 (MIG); CXCR3 (GPR9/CKR-L2); CXCR4; CXCR6 (TYMSTR/STRL33/Bonzo); CYB5; CYC1; CYSLTR1; DAB21P; DES; DKFZp451J0118; DNCL1; DPP4; E2F1; ECGF1; EDG1; EFNA1; EGFR, EFNA3; EFNB2; EGF; EGFR; ELAC2; ENG; ENO1; ENO2; ENO3; EPHB4; EPO; ERBB2 (Her-2); EREG; ERK8; ESR1; ESR2; F3 (TF); FADD; FasL; FASN; FCER1A; FCER2; FCGR3A; FGF; FGF1 (aFGF); FGF10; FGF1; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR3; FIGF (VEGFD); FIL1 (EPSILON); FIL1 (ZETA); FLJ12584; FLJ25530; FLRT1 (fibronectin); FLT1; FOS; FOSL1 (FRA-1); FY (DARC); GABRP (GABAa); GAGEB1; GAGEC1; GALNAC4S-6ST; GATA3; GDF5; GFI1; GGT1; GITR; GM-CSF; GNAS1; GNRH1; GPR2 (CCR10); GPR31; GPR44; GPR81 (FKSG80); GRCC10 (C10); GRP; GSN (Gelsolin); GSTP1; HAVCR2; HDAC4; HDAC5; HDAC7A; HDAC9; HER; HGF; HIF1A; HIP1; histamine and histamine receptors; HLA-A; HLA-DRA; HM74; HMOX1; HUMCYT2A; ICEBERG; ICOSL; ID2; IFN-α; IFNA1; IFNA2; IFNA4; IFNA5; IFNA6; IFNA7; IFNB1; IFNgamma; IFNW1; IGBP1; IGF; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; IL1F9; IL1HY1; IL1R1; IL1R2; IL1RAP; IL1RAPL1; IL1RAPL2; IL1RL1; IL1RL2 IL1RN; IL2; IL20; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; IRAK1; IRAK2; ITGA1; ITGA2; ITGA3; ITGA6 (a6 integrin); ITGAV; ITGB3; ITGB4 (b 4 integrin); JAG1; JAK1; JAK3; JUN; K6HF; KAI1; KDR; KITLG; KLF5 (GC Box BP); KLF6; KLK10; KLK12; KLK13; KLK14; KLK15; KLK3; KLK4; KLK5; KLK6; KLK9; KRT1; KRT19 (Keratin 19); KRT2A; KRTHB6 (hair-specific type II keratin); LAMAS; LEP (leptin); Lingo-p75; Lingo-Troy; LPS; LTA (TNF-b); LTB; LTB4R (GPR16); LTB4R2; LTBR; MACMARCKS; MAG or Omgp; MAP2K7 (c-Jun); MDK; MIB1; midkine; MIF; MIP-2; MKI67 (Ki-67); MMP2; MMP9; MS4A1; MSMB; MT3 (metallothionectin-III); MTSS1; MUC1 (mucin); MYC; MYD88; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NR0B1; NROB2; NR1D1; NR1D2; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZ1; OPRD1; P2RX7; PAP; PART1; PATE; PAWR; PCA3; PCNA; PDGF; PDGFA; PDGFB; PECAM1; PF4 (CXCL4); PGF; PGR; phosphacan; PIAS2; PIK3CG; PLAU (uPA); PLG; PLXDC1; PPBP (CXCL7); PPID; PR1; PRKCQ; PRKD1; PRL; PROC; PROK2; PSAP; PSCA; PTAFR; PTEN; PTGS2 (COX-2); PTN; RAC2 (p21Rac2); RARB; RGS1; RGS13; RGS3; RNF110 (ZNF144); ROBO2; S100A2; SCGB1D2 (lipophilin B); SCGB2A1(mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPP1; SPRR1B (Spr1); ST6GAL1; STAB1; STATE; STEAP; STEAP2; TB4R2; TBX21; TCP10; TDGF1; TEK; TGFA; TGFB1; TGFB1I1; TGFB2; TGFB3; TGFB1; TGFBR1; TGFBR2; TGFBR3; TH1L; THB S1 (thrombospondin-1); THBS2; THBS4; THPO; TIE (Tie-1); TIGIT; TIMP3; tissue factor; TLR10; TLR2; TLR3; TLR4; TLR5; TLR6; TLR7; TLR8; TLR9; TNF; TNF-α; TNFAIP2 (B94); TNFAIP3; TNFRSF11A; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNF SF13 (April); TNFSF13B; TNF SF14 (HVEM-L); TNF SF15 (VEGI); TNFSF18; TNFSF4 (0X40 ligand); TNFSF5 (CD40 ligand); TNFSF6 (FasL); TNFSF7 (CD27 ligand); TNFSF8 (CD30 ligand); TNFSF9 (4-1BB ligand); TOLLIP; Toll-like receptors; TOP2A (topoisomerase Iia); TP53; TPM1; TPM2; TRADD; TRAF1; TRAF2; TRAF3; TRAF4; TRAF5; TRAF6; TREM1; TREM2; TRPC6; TSLP; TWEAK; VEGF; VEGFB; VEGFC; versican; VHL C5; VLA-4; XCL1 (lymphotactin); XCL2 (SCM-1b); XCR1 (GPR5/CCXCR1); YY1; and ZFPM2.

9. Compositions

In another aspect, compositions comprising the monovalent or divalent binding proteins described herein are provided, as well as methods of using such compositions for diagnostic purposes or to treat a disease in a patient. The compositions provided herein contain one or more of the monovalent or divalent binding proteins disclosed herein, formulated together with a carrier (e.g., a “pharmaceutically acceptable carrier”). As used herein, “pharmaceutically acceptable carrier” includes any and all solvents, dispersion media, coatings, antibacterial and antifungal agents, isotonic and absorption delaying agents, and the like that are physiologically compatible. The carrier can be a solvent or dispersion medium containing, for example, water, ethanol, polyol (for example, glycerol, propylene glycol, and liquid polyethylene glycol, and the like), and suitable mixtures thereof. Saline solutions and aqueous dextrose and glycerol solutions can be employed as liquid carriers, particularly for injectable solutions. Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any excipient, diluent or agent is incompatible with the active compound, use thereof in the pharmaceutical compositions provided herein is contemplated. Supplementary active compounds (e.g., additional anti-cancer agents) can also be incorporated into the compositions.

Therapeutic compositions typically must be sterile and stable under the conditions of manufacture and storage. The composition can be formulated as a solution, microemulsion, liposome, or other ordered structure suitable to high drug concentration. The composition, if desired, can also contain minor amounts of wetting or solubility enhancing agents, stabilizers, preservatives, or pH buffering agents. In many cases, it will be useful to include isotonic agents, for example, sodium chloride, sugars, polyalcohols such as mannitol, sorbitol, glycerol, propylene glycol, and liquid polyethylene glycol in the composition. Prolonged absorption of the injectable compositions can be brought about by including in the composition an agent that delays absorption, for example, monostearate salts and gelatin

In the context of treating a disease in a patient, preferably, the carrier is suitable for intravenous, intramuscular, subcutaneous, parenteral, spinal or epidermal administration (e.g., by injection or infusion). Depending on the route of administration, the monovalent or divalent binding protein may be coated in a material to protect them from the action of acids and other natural conditions that may inactivate proteins. For example, the monovalent or divalent binding protein may be administered to a patient in an appropriate carrier, for example, in liposomes, or a diluent. Pharmaceutically acceptable diluents include saline and aqueous buffer solutions. Liposomes include water-in-oil-in-water CGF emulsions, as well as conventional liposomes. The composition can be administered by a variety of methods known in the art. As will be appreciated by the skilled artisan, the route and/or mode of administration will vary depending upon the desired results.

Pharmaceutical compositions may be administered alone or in combination therapy, i.e., combined with other agents. For example, the combination therapy can include a monovalent or divalent binding protein described herein with at least one additional therapeutic agent (e.g., an anti-cancer agent). Pharmaceutical compositions can also be administered in conjunction with an anti-cancer treatment modality, such as radiation therapy and/or surgery.

10. Nucleic Acids, Expression Vectors, Host Cells, and Methods of Production

Further provided herein are nucleic acids, e.g., DNA and RNA, encoding the monovalent and divalent binding proteins described herein. Exemplary nucleotide sequences provided herein are those encoding the encoding sequences set forth in the Figures.

Nucleic acids, e.g., DNA, that comprise a nucleotide sequence that is at least about 70%, 75%, 80%, 90%, 95%, 97%, 98% or 99% identical to a nucleotide sequence encoding a polypeptide described herein or a nucleotide sequence set forth herein are also encompassed herein. Such nucleotide sequences may encode a protein set forth herein or may encode a protein that is at least 70%, 75%, 80%, 85%, 90%, 95%, 97%, 98% or 99% identical or similar to a protein set forth herein or a portion thereof (e.g., a domain), such as an amino acid sequence set forth in any one of the Figures.

In certain embodiments, a nucleotide sequence encoding a polypeptide is linked to a sequence that enhances or promotes the expression of the nucleotide sequence in a cell to produce a protein. Such nucleic acids may be encompassed within a vector, e.g., an expression vector.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” as used herein refers to polynucleotide sequences which are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences.

For the purposes of being secreted, the constructs described herein preferably comprises a signal sequence, which is normally cut off after secretion to provide a mature polypeptide. “Leader sequence,” “signal peptide,” or a “secretory leader,” are terms are used interchangeably, contains a sequence comprising amino acid residues that directs the intracellular trafficking of the polypeptide to which it is a part. Polypeptides contain secretory leaders, signal peptides or leader sequences, typically at their N-terminus. These polypeptides may also contain cleavage sites where the leader sequences may be cleaved from the rest of the polypeptides by signal endopeptidases. Such cleavage results in the generation of mature polypeptides. Cleavage typically takes place during secretion or after the intact polypeptide has been directed to the appropriate cellular compartment. Exemplary signal sequences are disclosed above in the “Exemplary Sequences” section.

“Transformation” refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. Preferred eukaryotic cells include protist, fungal, plant and animal cells. In one embodiment, HEK293 cells and CHO cells are used as host cells. Expression in NSO cells is described by, e.g., Barnes, L. M., et al, Cytotechnology 32 109-123 (2000); Barnes, L. M., et al., Biotech. Bioeng. 73 261-270 (2001). Transient expression is described by, e.g., Durocher, Y., et al., Nucl. Acids. Res. 30 E9 (2002). Cloning of variable domains is described by Orlandi, R., et al., Proc. Natl. Acad. Sci. USA 86 3833-3837 (1989); Carter, P., et al., Proc. Natl. Acad. Sci. USA 89 4285-4289 (1992); and Norderhaug, L., et al., J. Immunol. Methods 204 77-87 (1997). An exemplary transient expression system (HEK 293) is described by Schlaeger, E.-J., and Christensen, K., in Cytotechnology 30 71-83 (1999) and by Schlaeger, E.-J., in J. Immunol. Methods 194 191-199 (1996).

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)), which is incorporated herein by reference for any purpose.

Methods for recombinant production are widely known in the state of the art and comprise protein expression in prokaryotic and eukaryotic cells with subsequent isolation of the antibody and usually purification to a pharmaceutically acceptable purity. For the expression of the binding proteins in a host cell, nucleic acids encoding the respective polypeptides are inserted into expression vectors by standard methods. Expression is performed in appropriate prokaryotic or eukaryotic host cells (such as CHO cells, NSO cells, SP2/0 cells, HEK293 cells, COS cells, PER.C6 cells, yeast, or E. coli cells), and the binding protein is recovered from the cells (supernatant or cells after lysis). General methods for recombinant production of antibodies are well-known in the state of the art and described, for example, in the review articles of Makrides, S. C., Protein Expr. Purif 17 183-202 (1999); Geisse, S., et al, Protein Expr. Purif. 8 271-282 (1996); Kaufman, R. J., MoI. Biotechnol. 16 151-161 (2000); Werner, R. G., Drug Res. 48 870-880 (1998).

The monovalent and divalent binding proteins may be suitably separated from the culture medium by conventional immunoglobulin purification procedures. Purification can be performed in order to eliminate cellular components or other contaminants, e.g. other cellular nucleic acids or proteins, by standard techniques, including alkaline/SDS treatment, CsCl banding, column chromatography, agarose gel electrophoresis, and others well known in the art. See Ausubel, F., et al., ed. Current Protocols in Molecular Biology, Greene Publishing and Wiley Interscience, New York (1987). Different methods are well established and widespread used for protein purification, such as affinity chromatography with microbial proteins (e.g. protein A or protein G affinity chromatography), ion exchange chromatography (e.g. cation exchange (carboxylmethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and electrophoretical methods (such as gel electrophoresis, capillary electrophoresis) (Vijayalakshmi, M. A. Appl. Biochem. Biotech. 75 93-102 (1998)). DNA and RNA encoding the monovalent and divalent binding proteins are readily isolated and sequenced using conventional procedures.

In another aspect, methods of producing the monovalent or divalent binding proteins described herein are provided comprising: culturing a host cell in culture medium under conditions wherein the nucleic acid sequence is expressed, thereby producing the binding protein; and recovering the binding protein from the host cell or culture medium. In another embodiment, the monovalent or divalent binding protein is coexpressed with a light chain.

11. Kits

Also provided are kits (e.g., diagnostic kits) comprising one or more of the monovalent or divalent binding proteins described herein. Such kits are useful in the methods for diagnosis, detection or monitoring described herein. The kits may include a label indicating the intended use of the contents of the kit and optionally including instructions for use of the kit in diagnosing or treating a disease. These kits may optionally comprise a detectable label, e.g. indicator enzymes, radiolabels, fluorophores, or paramagnetic particles. Kits may include informative pamphlets, for example, pamphlets informing one how to use reagents to practice a method disclosed herein. The term “pamphlet” includes any writing, marketing materials or recorded material supplied on or with the kit, or which otherwise accompanies the kit.

12. Methods of Detection and Monitoring

Further provided are methods of detecting the presence or absence of a target molecule of interest in a biological sample comprising (A) contacting a sample with any of the monovalent or divalent heavy chain binding proteins described herein, wherein the binding protein specifically binds the target molecule of interest, and (B) detecting the presence or absence of at least one complex comprising the binding protein and target molecule of interest. The target molecule of interest can include any suitable molecule of interest and include, but are not limited to, the exemplary molecules disclosed above in the “Target Molecules” section.

The term “sample”, as used herein, is used in its broadest sense. A “biological sample”, as used herein, includes, but is not limited to, any quantity of a substance from a living thing or formerly living thing. Such living things include, but are not limited to, humans, mice, rats, monkeys, dogs, rabbits and other animals. In one embodiment, the biological sample is from a human patient. Such substances include, but are not limited to, blood, serum, urine, synovial fluid, cells, organs, tissues, bone marrow, lymph nodes and spleen. In one embodiment, the sample comprising cells, such as cancer cells.

Also provided are methods of monitoring (e.g., determining) the regression, progression, course and/or onset, of a disease (such as cancer) comprising detection and/or determination of quantity of cells in a biological sample which express a target molecule of interest. Such methods can be used to detect whether a subject has a disease, is at (increased) risk of developing a disease, or whether a treatment regimen is efficient.

The methods of monitoring preferably comprise a detection of and/or determination of the quantity of the cells which express the target molecule of interest in a first sample at a first point in time and in a further sample at a second point in time, wherein the regression, progression, course and/or onset of a disease may be determined by comparing the two samples.

Typically, the level of cells expressing a target molecule of interest in a biological sample is compared to a reference level, wherein a deviation from said reference level is indicative of the presence and/or stage of a disease in a subject. The reference level may be a level as determined in a control sample (e.g., from a healthy tissue or subject, in particular a patient without a disease) or a median level from healthy subjects. A “deviation” from said reference level designates any significant change, such as an increase by at least 10%, 20%, or 30%, preferably by at least 40% or 50%, or even more.

In one embodiment, the presence of cells expressing a target molecule of interest and/or a quantity of the target-expressing cells which is increased compared to a reference level, e.g., compared to a patient without a disease, indicates the presence of or risk for (i.e., a potential for a development of) a disease in the patient. “Being at risk” means that a subject, i.e., a patient, is identified as having a higher than normal chance of developing a disease compared to the general population. In addition, a subject who has had, or who currently has, a disease, is a subject who has an increased risk for developing a disease, as such a subject may continue to develop a disease.

In another embodiment, a quantity of cells expressing a target molecule of interest which is decreased compared to a biological sample taken earlier from a patient may indicate a regression, a positive course, e.g., a successful treatment, or a reduced risk for an onset of a disease in a patient.

In another embodiment, a quantity of cells expressing a target molecule of interest which is increased compared to a biological sample taken earlier from a patient may indicate a progression, a negative course, e.g., an unsuccessful treatment, recurrence or metastatic behaviour, an onset or a risk for an onset of a disease in said patient.

Detection of the complex can be carried out in a number of ways, including but not limited to, ELISA, competitive binding assays, Western blot, flow cytometry, lateral flow, or Immunohistochemistry (IHC). In one embodiment, the monovalent or divalent heavy chain binding protein that specifically binds a target molecule of interest is directly or indirectly bound to a label that provides for detection, e.g. indicator enzymes, radiolabels, fluorophores, or paramagnetic particles. For example, the monovalent or divalent heavy chain binding protein may be directly or indirectly bound to a label that functions to: (i) provide a detectable signal; (ii) interact with a second label to modify the detectable signal provided by the first or second label, e.g. FRET (Fluorescence Resonance Energy Transfer); (iii) affect mobility, e.g., electrophoretic mobility, by charge, hydrophobicity, shape, or other physical parameters, or (iv) provide a capture moiety, e.g., affinity, antibody/antigen, or ionic complexation. Suitable as label are structures, such as fluorescent labels, luminescent labels, chromophore labels, radioisotopic labels, isotopic labels, preferably stable isotopic labels, isobaric labels, enzyme labels, particle labels, in particular metal particle labels, magnetic particle labels, polymer particle labels, small organic molecules such as biotin, ligands of receptors or binding molecules such as cell adhesion proteins or lectins, label-sequences comprising nucleic acids and/or amino acid residues which can be detected by use of binding agents, etc. Labels comprise, in a nonlimiting manner, barium sulfate, iocetamic acid, iopanoic acid, calcium ipodate, sodium diatrizoate, meglumine diatrizoate, metrizamide, sodium tyropanoate and radio diagnostic, including positron emitters such as fluorine-18 and carbon-11, gamma emitters such as iodine-123, technetium-99m, iodine-131 and indium-111, nuclides for nuclear magnetic resonance, such as fluorine and gadolinium.

In one embodiment, the target molecule is detected via IHC. IHC refers to the process of detecting antigens (e.g., proteins) in cells of a tissue section. Immunohistochemical staining is widely used in the diagnosis of abnormal cells, such as those found in cancerous tumors. Visualising a target molecule can be accomplished in a number of ways. In the most common instance, the binding protein is conjugated to an enzyme, such as peroxidase, that can catalyse a colour-producing reaction. Alternatively, the binding protein can also be tagged to a fluorophore, such as fluorescein or rhodamine. Preparation of the sample is critical to maintain cell morphology, tissue architecture and the antigenicity of target epitopes. This requires proper tissue collection, fixation and sectioning. Paraformaldehyde is usually used with fixation. Depending on the purpose and the thickness of the experimental sample, either thin (about 4-40 .mu.m) sections are sliced from the tissue of interest, or if the tissue is not very thick and is penetrable it is used whole. The slicing is usually accomplished through the use of a microtome, and slices are mounted on slides. The sample may require additional steps to make the epitopes available for the binding protein, including deparaffinization and antigen retrieval. Detergents like Triton X-100 are generally used in IHC to reduce surface tension, allowing less reagent to be used to achieve better and more even coverage of the sample.

13. Methods of Increasing Binding Affinity

The monovalent and divalent heavy chain binding proteins described herein are engineered to have increased binding specificity and affinity. Accordingly, provided are methods of increasing (e.g., synergistically increasing) the binding affinity of a binding protein (e.g., a monovalent or divalent binding protein) by linking one or more additional heavy chain variable regions to the amino terminus of the binding protein. A “synergistic increase” in binding of a given binding protein refers to an increase in binding affinity that is greater than the sum of the individual binding affinities (greater than two fold) of the same binding protein (i.e., having the same variable region or variable region that binds to the same epitope or target).

In one embodiment, the additional heavy chain variable region is the same as (e.g., has the same amino acid sequence) the heavy chain variable region of the binding protein (e.g., VR1 and VR1). In another embodiment, the additional heavy chain variable region binds to the same epitope as the heavy chain variable region of the binding protein (e.g., VR1 and VR2). In another embodiment, the additional heavy chain variable region binds to the same target molecule as the heavy chain variable region of the binding protein (e.g., VR1 and VR2).

In one embodiment, the binding protein is a single chain binding protein. In another embodiment, the binding protein is a monovalent binding protein. In another embodiment, the binding protein is a single chain monovalent binding protein. In another embodiment, the binding protein is a two chain binding protein. In another embodiment, the binding protein is a two chain monovalent binding protein. In another embodiment, the binding protein is a divalent binding protein. In another embodiment, the binding protein is a two chain divalent binding protein. In another embodiment, the binding increases to subpicomolar levels.

In another embodiment, the synergistic increase is about a 5-fold, 10-fold, 15-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90, fold, 95-fold, 100-fold, 105-fold, 110-fold, 115-fold, 120-fold, 125-fold, 130-fold, 135-fold, 140-fold, 145-fold, 150-fold, 155-fold, 160-fold, 165-fold, 170-fold, 175-fold, 180-fold, 185-fold, 190-fold, 195-fold, 200-fold, 205-fold, 210-fold, 215-fold, 220-fold, 225-fold, 230-fold, 235-fold, 240-fold, 245-fold, 250-fold, 255-fold, 260-fold, 265-fold, 270-fold, 275-fold, 280-fold, 285-fold, 290-fold, 295-fold, 300-fold, 305-fold, 310-fold, 315-fold, 320-fold, 325-fold, 330-fold, 335-fold, 340-fold, 345-fold, 350-fold, 355-fold, 360-fold, 365-fold, 370-fold, 375-fold, 380-fold, 385-fold, 390-fold, 395-fold, 400-fold, 405-fold, 410-fold, 415-fold, 420-fold, 425-fold, 430-fold, 435-fold, 440-fold, 445-fold, 450-fold, 455-fold, 460-fold, 465-fold, 470-fold, 475-fold, 480-fold, 485-fold, 490-fold, 495-fold, 500-fold, 505-fold, 510-fold, 515-fold, 520-fold, 525-fold, 530-fold, 535-fold, 540-fold, 545-fold, 550-fold, 555-fold, 560-fold, 565-fold, 570-fold, 575-fold, 580-fold, 585-fold, 590-fold, 595-fold, or 600-fold increase. In one embodiment, the synergistic increase is a 20-fold increase. In another embodiment, the synergistic increase is a 454-fold increase.

In another embodiment, the binding protein is as depicted in FIG. 1. In another embodiment, the binding protein is as depicted in FIG. 2. In another embodiment, the binding protein is as depicted in FIG. 3. In another embodiment, the binding protein is as depicted in FIG. 4. In another embodiment, the binding protein is as depicted in FIG. 5. In another embodiment, the binding protein is as depicted in FIG. 6. In another embodiment, the binding protein is as depicted in FIG. 7. In another embodiment, the binding protein is as depicted in FIG. 8.

In another embodiment, the first variable domain and CH1 domain are linked to the second variable domain by a linker comprising or consisting of the amino acid sequence set forth in SEQ ID NO:10.

In another embodiment, the binding protein is a two chain monovalent heavy chain binding protein, wherein the first and second heavy chain variable domains are each paired (e.g., associated) with a light chain, e.g., via a disulfide bond. In one embodiment, the light chains of the two chain monovalent heavy chain binding protein are not linked to one another.

14. Methods of Diagnosis and Treatment

Also provided are methods of treating a patient (e.g., a human patient) having a disease (e.g., a cancer, infectious disease, inflammatory or autoimmune disorder) by administering an effective amount of any of the monovalent or divalent heavy chain binding proteins described herein, thereby treating the disease. In one embodiment, the method comprises administering an additional therapeutic agent to the patient.

The terms “treat,” “treating,” and “treatment,” as used herein, refer to therapeutic or preventative measures described herein. The methods of “treatment” employ administration to a patient the monovalent or divalent heavy chain binding proteins disclosed herein in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” refers to the amount of a therapy which is sufficient to reduce or ameliorate the severity and/or duration of a disease or one or more symptoms thereof, prevent the advancement of a disease, cause regression of a disease, prevent the recurrence, development, onset or progression of one or more symptoms associated with a disease, detect a disease, or enhance or improve the prophylactic or therapeutic effect(s) of another therapy (e.g., prophylactic or therapeutic agent).

In addition, methods of diagnosing a patient as having a disease characterized by a target molecule of interest are provided, comprising (A) contacting a sample obtained from the patient with any of the monovalent or divalent heavy chain binding proteins described herein, wherein the binding protein binds the target molecule of interest, and (B) diagnosing the patient as having the disease based on detection of at least one complex comprising the binding protein and target molecule of interest. The target molecule of interest can include any suitable molecule of interest and include, but are not limited to, the exemplary molecules disclosed above in the “Target Molecules” section.

In one embodiment, the disease is cancer. Exemplary cancers include, but are not limited to melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

In another embodiment, the disease is an autoimmune disease. An “autoimmune disease” herein is a disease or disorder arising from and directed against an individual's own tissues or a co-segregate or manifestation thereof or resulting condition therefrom. Examples of autoimmune diseases or disorders include, but are not limited to arthritis (rheumatoid arthritis such as acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, and ataxic sclerosis, inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), pyoderma gangrenosum, erythema nodosum, primary sclerosing cholangitis, episcleritis), respiratory distress syndrome, including adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, allergic conditions, allergic reaction, eczema including allergic or atopic eczema, asthma such as asthma bronchiale, bronchial asthma, and auto-immune asthma, conditions involving infiltration of T cells and chronic inflammatory responses, chronic pulmonary inflammatory disease, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis including lymphomatoid granulomatosis, Wegener's granulomatosis, agranulocytosis, vasculitides, including vasculitis (including large vessel vasculitis (including polymyalgia rheumatica and giant cell (Takayasu's) arteritis), medium vessel vasculitis (including Kawasaki's disease and polyarteritis nodosa), microscopic polyarteritis, CNS vasculitis, necrotizing, cutaneous, or hypersensitivity vasculitis, systemic necrotizing vasculitis, and ANCA-associated vasculitis, such as Churg-Strauss vasculitis or syndrome (CSS)), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, immune complex nephritis, antibody-mediated nephritis, neuromyelitis optica, polyneuropathies, chronic neuropathy such as IgM polyneuropathies or IgM-mediated neuropathy, thrombocytopenia (as developed by myocardial infarction patients, for example), including thrombotic thrombocytopenic purpura (TTP) and autoimmune or immune-mediated thrombocytopenia such as idiopathic thrombocytopenic purpura (ITP) including chronic or acute ITP, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barré-syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis (e.g., benign monoclonal gammopathy and monoclonal gammopathy of undetermined significance, MGUS), peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine ophthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia areata, CREST syndrome (calcinosis, Raynaud's phenomenon, esophageal dysmotility, sclerodactyly, and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endomyocardial fibrosis, diffuse interstitial pulmonary fibrosis, interstitial lung fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired spenic atrophy, infertility due to antispermatozoan antibodies, non-malignant thymoma, vitiligo, SCID and Epstein-Barr virus-associated diseases, acquired immune deficiency syndrome (AIDS), parasitic diseases such as Leishmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

In another embodiment, the disease is an infectious disease. In one embodiment, the infectious disease relates to an agent selected from the group consisting of: a virus, a bacterium, a fungus, and a protozoan parasite. Exemplary infectious diseases include, but are not limited to human immunodeficiency viruses, hepatitis viruses class A, B and C, Eppstein Barr virus, human cytomegalovirus, human papilloma viruses, herpes viruses, leishmaniasis, toxoplasmosis, cryptosporidiosis, sleeping sickness, and malaria.

All references cited throughout this application, for example patent documents including issued or granted patents or equivalents; patent application publications; and non-patent literature documents or other source material; are hereby incorporated by reference herein in their entireties, as though individually incorporated by reference. Any sequence listing and sequence listing information is considered part of the disclosure herewith.

The following examples should not be construed as limiting the scope of this disclosure.

### EXAMPLES

### Example 1: Generation of Heavy Chain Constructs for 2-Chain and Single Chain Antibodies

Five full length heavy chain constructs comprising “stacked” variable regions were generated as follows. The basic expression construct for the “stacked” heavy chain constructs, included (a) a signal peptide, (b) the variable domain and CH1 domain of a first heavy chain (VH1-CH1), (c) a linker consisting of a two-amino acid repeat (PQ Linker), and (d) the variable domain and CH1 domain of the second heavy chain (VH2-CH1), as well as (e) a hinge region, CH2 domain, and CH3 domain.

Heavy Chain Construct 1 (SEQ ID NOS 19-20, respectively, in order of appearance) contained a heavy chain sequence linked and in-frame with a second heavy chain sequence and is set forth below. Both heavy chain sequences were developed from a single 2-chain antibody (Antibody 1). Bold and underlined DNA sequences are restriction sites used for subcloning to build the constructs. The protein sequence in [symbol] is the signal peptide. The underlined sequences are the two variable domains in tandem of Antibody 1. The PQ Linker is highlighted.

```
                                                          gctagcgacgctcaccatggagactgggctg

[symbol]

cgctggcttctcctggtcgctgtgctcaaaggtgtccagtgtcagtcggtggaggagtcc
  R W L L L V A V L K G V Q C Q S V E E S
gggggtcgcctggtcacgcctgggacacccctgacactcacctgcacagtctctggattc
  G G R L V T P G T P L T L T C T V S G F
tccctcaataccaatgcaataaggtgggtccgccaggctccagggaaggggctggaatgg
  S L N T N A I R W V R Q A P G K G L E W
atcggagccattgatgctagtggtaacacatactacgcgagctgggcgaaaggccgattc
  I G A I D A S G N T Y Y A S W A K G R F
accatctccaaaacctcgaccacggtggatctgaaaatcaccagtccgacaaccgaggac
  T I S K T S T T V D L K I T S P T T E D
acggccacctatttctgtaacagatttaatactgccaacatctggggcccaggcaccctg
  T A T Y F C N R F N T A N I W G P G T L
gtcaccgtctcctcagggcaacctaaggctccatcagtcttcccactggccccctgctgc
  V T V S S G Q P K A P S V F P L A P C C
ggggacacacccagctccacggtgaccctgggctgcctggtcaaaggctacctcccggag
  G D T P S S T V T L G C L V K G Y L P E
ccagtgaccgtgacctggaactcgggcaccctcaccaatggggtacgcaccttcccgtcc
  P V T V T W N S G T L T N G V R T F P S
gtccggcagtcctcaggcctctactcgctgagcagcgtggtgagcgtgacctcaagcagc
  V R Q S S G L Y S L S S V V S V T S S S
cagcccgtcacctgcaacgtggcccacccagccaccaacaccaaagtggacaagaccgtt
  Q P V T C N V A H P A T N T K V D K T V
gcgccctcgacatgcagcaagcccatgtgcccaccccctgaactcctgggcggccgccct
  A P S T C S K P M C P P P E L L G G R P
caaccccaaccacaaccgcaacctcagccccagccacagccgcagccgcagccacaaccc
  Q P Q P Q P Q P Q P Q P Q P Q P Q P Q P
cagcctcaaccacaaccccagcctcaacctcagtctagacagtoggtggaggagtccggg
  Q P Q P Q P Q P Q P Q S R Q S V E E S G
ggtcgcctggtcacgcctgggacacccctgacactcacctgcacagtctctggattctcc
  G R L V T P G T P L T L T C T V S G F S
ctcaataccaatgcaataaggtgggtccgccaggctccagggaaggggctggaatggatc
  L N T N A I R W V R Q A P G K G L E W I
ggagccattgatgctagtggtaacacatactacgcgagctgggcgaaaggccgattcacc
  G A I D A S G N T Y Y A S W A K G R F T
atctccaaaacctcgaccacggtggatctgaaaatcaccagtccgacaaccgaggacacg
  I S K T S T T V D L K I T S P T T E D T
gccacctatttctgtaacagatttaatactgccaacatctggggcccaggcaccctggtc
  A T Y F C N R F N T A N I W G P G T L V
accgtctcctcagggcaacctaaggctccatcagtcttcccactggccccctgctgcggg
  T V S S G Q P K A P S V F P L A P C C G
gacacacccagctccacggtgaccctgggctgcctggtcaaaggctacctcccggagcca
  D T P S S T V T L G C L V K G Y L P E P
gtgaccgtgacctggaactcgggcaccctcaccaatggggtacgcaccttcccgtccgtc
  V T V T W N S G T L T N G V R T F P S V
cggcagtcctcaggcctctactcgctgagcagcgtggtgagcgtgacctcaagcagccag
  R Q S S G L Y S L S S V V S V T S S S Q
cccgtcacctgcaacgtggcccacccagccaccaacaccaaagtggacaagaccgttgcg
  P V T C N V A H P A T N T K V D K T V A
ccctcgacatgcagcaagcccacgtgcccaccccctgaactcctggggggaccgtctgtc
  P S T C S K P T C P P P E L L G G P S V
ttcatcttccccccaaaacccaaggacaccctcatgatctcacgcacccccgaggtcaca
  F I F P P K P K D T L M I S R T P E V T
tgcgtggtggtggacgtgagccaggatgaccccgaggtgcagttcacatggtacataaac
  C V V V D V S Q D D P E V Q F T W Y I N
aacgagcaggtgcgcaccgcccggccgccgctacgggagcagcagttcaacagcacgatc
  N E Q V R T A R P P L R E Q Q F N S T I
cgcgtggtcagcaccctccccatcgcgcaccaggactggctgaggggcaaggagttcaag
  R V V S T L P I A H Q D W L R G K E F K
tgcaaagtccacaacaaggcactcccggcccccatcgagaaaaccatctccaaagccaga
  C K V H N K A L P A P I E K T I S K A R
gggcagcccctggagccgaaggtctacaccatgggccctccccgggaggagctgagcagc
  G Q P L E P K V Y T M G P P R E E L S S
aggtcggtcagcctggcctgcatgatcaacggcttctacccttccgacatctcggtggag
  R S V S L A C M I N G F Y P S D I S V E
tgggagaagaacgggaaggcagaggacaactacaagaccacgccggccgtgctggacagc
  W E K N G K A E D N Y K T T P A V L D S
gacggctcctacttcctctacagcaagctctcagtgcccacgagtgagtggcagcggggc
  D G S F L Y Y S K L S V P T S E W Q R G
gacgtcttcacctgctccgtgatgcacgaggccttgcacaaccactacacgcagaagtcc
  D V F T C S V M H E A L H N H Y T Q K S
atctoccgctctccgggtaaatgaggatcc
  I S R S P G K -
```

Heavy Chain Construct 2 (SEQ ID NOS 22-23, respectively, in order of appearance) contained a heavy chain sequence linked and in-frame with a second heavy chain sequence and is set forth below. Both heavy chain sequences were developed from a single 2-chain antibody (Antibody 2). Bold and underlined DNA sequences are restriction sites used for subcloning to build the constructs. The protein sequence in [symbol] is the signal peptide. The underlined sequences are the two variable domains in tandem of Antibody 1. The PQ Linker is highlighted.

```
                                                          gctagcgacgctcaccatggagactgggctg

[symbol]

cgctggcttctcctggtcgctgtgctcaaaggtgtccagtgtcagtcgctggaggagtcc [symbol]
  Q S L E E S
gggggtcgcctggtctcgcctgggacacccctgacactcacctgcaccgcctctggattc
  G G R L V S P G T P L T L T C T A S G F
tccctcagtagcaatgcaatgggctgggtccgccaggctccaggggaggggctggaatac
  S L S S N A M G W V R Q A P G E G L E Y
atcggaatcattaggcctggtggtagcacatactacgcgagctgggcgaaaggccgattc
  I G I I R P G G S T Y Y A S W A K G R F
accatctccaaaacctcgtcgaccacggtggatctgaaaatgaccagtctgacaaccgag
  T I S K T S S T T V D L K M T S L T T E
gacacggccacctatttctgtgccagagcctgggacatctggggcccaggcaccctggtc
  D T A T Y F C A R A W D I W G P G T L V
accgtctccttagggcaacctaaggctccatcagtcttcccactggccccctgctgcggg
  T V S L G Q P K A P S V F P L A P C C G
gacacacccagctccacggtgaccctgggctgcctggtcaaaggctacctcccggagcca
  D T P S S T V T L G C L V K G Y L P E P
gtgaccgtgacctggaactcgggcaccctcaccaatggggtacgcaccttcccgtccgtc
  V T V T W N S G T L T N G V R T F P S V
cggcagtcctcaggcctctactcgctgagcagcgtggtgagcgtgacctcaagcagccag
  R Q S S G L Y S L S S V V S V T S S S Q
cccgtcacctgcaacgtggcccacccagccaccaacaccaaagtggacaagaccgttgcg
  P V T C N V A H P A T N T K V D K T V A
ccctcgacatgcagcaagcccacgtgcccaccccctgaactcctgggcggccgccctcaa
  P S T C S K P T C P P P E L L G G R P Q
ccccaaccacaaccgcaacctcagccccagccacagccgcagccgcagccacaaccccag
  P Q P Q P Q P Q P Q P Q P Q P Q P Q P Q
cctcaaccacaaccccagcctcaacctcag [symbol] cagtcgctggaggagtccgggggt
  P Q P Q P Q P Q P Q S R Q S L E E S G G
cgcctggtctcgcctgggacacccctgacactcacctgcaccgcctctggattctccctc
  R L V S P G T P L T L T C T A S G F S L
agtagcaatgcaatgggctgggtccgccaggctccaggggaggggctggaatacatcgga
  S S N A M G W V R Q A P G E G L E Y I G
atcattaggcctggtggtagcacatactacgcgagctgggcgaaaggccgattcaccatc
  I I R P G G S T Y Y A S W A K G R F T I
tccaaaacctcgtcgaccacggtggatctgaaaatgaccagtctgacaaccgaggacacg
  S K T S S T T V D L K M T S L T T E D T
gccacctatttctgtgccagagcctgggacatctggggcccaggcaccctggtcaccgtc
  A T Y F C A R A W D I W G P G T L V T V
tccttagggcaacctaaggctccatcagtcttcccactggccccctgctgcggggacaca
  S L G Q P K A P S V F P L A P C C G D T
cccagctccacggtgaccctgggctgcctggtcaaaggctacctcccggagccagtgacc
  P S S T V T L G C L V K G Y L P E P V T
gtgacctggaactcgggcaccctcaccaatggggtacgcaccttcccgtccgtccggcag
  V T W N S G T L T N G V R T F P S V R Q
tcctcaggcctctactcgctgagcagcgtggtgagcgtgacctcaagcagccagcccgtc
  S S G L Y S L S S V V S V T S S S Q P V
acctgcaacgtggcccacccagccaccaacaccaaagtggacaagaccgttgcgccctcg
  T C N V A H P A T N T K V D K T V A P S
acatgcagcaagcccacgtgcccaccccctgaactcctggggggaccgtctgtcttcatc
  T C S K P T C P P P E L L G G P S V F I
ttccccccaaaacccaaggacaccctcatgatctcacgcacccccgaggtcacatgcgtg
  F P P K P K D T L M I S R T P E V T C V
gtggtggacgtgagccaggatgaccccgaggtgcagttcacatggtacataaacaacgag
  V V D V S Q D D P E V Q F T W Y I N N E
caggtgcgcaccgcccggccgccgctacgggagcagcagttcaacagcacgatccgcgtg
  Q V R T A R P P L R E Q Q F N S T I R V
gtcagcaccctccccatcgcgcaccaggactggctgaggggcaaggagttcaagtgcaaa
  V S T L P I A H Q D W L R G K E F K C K
gtccacaacaaggcactcccggcccccatcgagaaaaccatctccaaagccagagggcag
  V H N K A L P A P I E K T I S K A R G Q
cccctggagccgaaggtctacaccatgggccctccccgggaggagctgagcagcaggtcg
  P L E P K V Y T M G P P R E E L S S R S
gtcagcctggcctgcatgatcaacggcttctacccttccgacatctcggtggagtgggag
  V S L A C M I N G F Y P S D I S V E W E
aagaacgggaaggcagaggacaactacaagaccacgccggccgtgctggacagcgacggc
  K N G K A E D N Y K T T P A V L D S D G
tcctacttcctctacagcaagctctcagtgcccacgagtgagtggcagcggggcgacgtc
  S F L Y Y S K L S V P T S E W Q R G D V
ttcacctgctccgtgatgcacgaggccttgcacaaccactacacgcagaagtccatctcc
  F T C S V M H E A L H N H Y T Q K S I S
cgctctccgggtaaatga [symbol]
  R S P G K -
```

Heavy Chain Construct 3 (SEQ ID NOS 25-26, respectively, in order of appearance) contained one heavy sequence from Antibody 1 linked and in-frame with a second heavy chain sequence from Antibody 2, as set forth below. Bold and underlined DNA sequences are restriction sites used for subcloning to build the constructs. The protein sequence in [symbol] is the signal peptide. The variable domain of Antibody 1 is underlined and the variable domain of Antibody 2 is in underlined italics. The PQ Linker is highlighted.

```
                                                          gctagcgacgctcaccatggagactgggctg

[symbol]

cgctggcttctcctggtcgctgtgctcaaaggtgtccagtgtcagtcggtggaggagtcc [symbol]
    Q S V E E S
gggggtcgcctggtcacgcctgggacacccctgacactcacctgcacagtctctggattc
  G G R L V T P G T P L T L T C T V S G F
tccctcaataccaatgcaataaggtgggtccgccaggctccagggaaggggctggaatgg
  S L N T N A I R W V R Q A P G K G L E W
atcggagccattgatgctagtggtaacacatactacgcgagctgggcgaaaggccgattc
  I G A I D A S G N T Y Y A S W A K G R F
accatctccaaaacctcgaccacggtggatctgaaaatcaccagtccgacaaccgaggac
  T I S K T S T T V D L K I T S P T T E D
acggccacctatttctgtaacagatttaatactgccaacatctggggcccaggcaccctg
  T A T Y F C N R F N T A N I W G P G T L
gtcaccgtctcctcagggcaacctaaggctccatcagtcttcccactggccccctgctgc
  V T V S S G Q P K A P S V F P L A P C C
ggggacacacccagctccacggtgaccctgggctgcctggtcaaaggctacctcccggag
  G D T P S S T V T L G C L V K G Y L P E
ccagtgaccgtgacctggaactcgggcaccctcaccaatggggtacgcaccttcccgtcc
  P V T V T W N S G T L T N G V R T F P S
gtccggcagtcctcaggcctctactcgctgagcagcgtggtgagcgtgacctcaagcagc
  V R Q S S G L Y S L S S V V S V T S S S
cagcccgtcacctgcaacgtggcccacccagccaccaacaccaaagtggacaagaccgtt
  Q P V T C N V A H P A T N T K V D K T V
gcgccctcgacatgcagcaagcccatgtgcccaccccctgaactcctgggcggccgccct
  A P S T C S K P M C P P P E L L G G R P
caaccccaaccacaaccgcaacctcagccccagccacagccgcagccgcagccacaaccc
  Q P Q P Q P Q P Q P Q P Q P Q P Q P Q P
cagcctcaaccacaaccccagcctcaacctcagtctagacagtcgctggaggagtccggg
  Q P Q P Q P Q P Q P Q S R Q S L E E S G
ggtcgcctggtctcgcctgggacacccctgacactcacctgcaccgcctctggattctcc
  G R L V S P G T P L T L T C T A S G F S
ctcagtagcaatgcaatgggctgggtccgccaggctccaggggaggggctggaatacatc
  L S S N A M G W V R Q A P G E G L E Y I
ggaatcattaggcctggtggtagcacatactacgcgagctgggcgaaaggccgattcacc
  G I I R P G G S T Y Y A S W A K G R F T
atctccaaaacctcgtcgaccacggtggatctgaaaatgaccagtctgacaaccgaggac
  I S K T S S T T V D L K M T S L T T E D
acggccacctatttctgtgccagagcctgggacatctggggcccaggcaccctggtcacc
  T A T Y F C A R A W D I W G P G T L V T
gtctccttagggcaacctaaggctccatcagtcttcccactggccccctgctgcggggac
  V S L G Q P K A P S V F P L A P C C G D
acacccagctccacggtgaccctgggctgcctggtcaaaggctacctcccggagccagtg
  T P S S T V T L G C L V K G Y L P E P V
accgtgacctggaactcgggcaccctcaccaatggggtacgcaccttcccgtccgtccgg
  T V T W N S G T L T N G V R T F P S V R
cagtcctcaggcctctactcgctgagcagcgtggtgagcgtgacctcaagcagccagccc
  Q S S G L Y S L S S V V S V T S S S Q P
gtcacctgcaacgtggcccacccagccaccaacaccaaagtggacaagaccgttgcgccc
  V T C N V A H P A T N T K V D K T V A P
tcgacatgcagcaagcccacgtgcccaccccctgaactcctggggggaccgtctgtcttc
  S T C S K P T C P P P E L L G G P S V F
atcttccccccaaaacccaaggacaccctcatgatctcacgcacccccgaggtcacatgc
  I F P P K P K D T L M I S R T P E V T C
gtggtggtggacgtgagccaggatgaccccgaggtgcagttcacatggtacataaacaac
  V V D V D S Q D D P E V Q F T W Y I N N
gagcaggtgcgcaccgcccggccgccgctacgggagcagcagttcaacagcacgatccgc
  E Q V R T A R P P L R E Q Q F N S T I R
gtggtcagcaccctccccatcgcgcaccaggactggctgaggggcaaggagttcaagtgc
  V V S T L P I A H Q D W L R G K E F K C
aaagtccacaacaaggcactcccggcccccatcgagaaaaccatctccaaagccagaggg
  K V H N K A L P A P I E K T I S K A R G
cagcccctggagccgaaggtctacaccatgggccctccccgggaggagctgagcagcagg
  Q P L E P K V Y T M G P P R E E L S S R
tcggtcagcctggcctgcatgatcaacggcttctacccttccgacatctcggtggagtgg
  S V S L A C M I N G F Y P S D I S V E W
gagaagaacgggaaggcagaggacaactacaagaccacgccggccgtgctggacagcgac
  E K N G K A E D N Y K T T P A V L D S D
ggctcctacttcctctacagcaagctctcagtgcccacgagtgagtggcagcggggcgac
  G S Y F L Y S K L S V P T S E W Q R G D
gtcttcacctgctccgtgatgcacgaggccttgcacaaccactacacgcagaagtccatc
  V F T C S N M H E A L H N H Y T Q K S I
tcccgctctccgggtaaatgaggatcc
  S R S P G K -
```

Heavy Chain Construct 4 (SEQ ID NOS 28-29, respectively, in order of appearance) contained one heavy sequence from Antibody 2 linked and in-frame with a second heavy chain sequence from Antibody 1, as set forth below. Bold and underlined DNA sequences are restriction sites used for subcloning to build the constructs. The protein sequence in [symbol] is the signal peptide. The variable domain of Antibody 2 is underlined and the variable domain of Antibody 1 is in underlined italics. The PQ Linker is highlighted.

```
                                                          gctagcgacgctcaccatggagactgggctg

[symbol]

cgctggcttctcctggtcgctgtgctcaaaggtgtccagtgtcagtcgctggaggagtcc [symbol]
  Q S L E E S
gggggtcgcctggtctcgcctgggacacccctgacactcacctgcaccgcctctggattc
  G G R L V S P G T P L T L T C T A S G F
tccctcagtagcaatgcaatgggctgggtccgccaggctccaggggaggggctggaatac
  S L S S N A M G W V R Q A P G E G L E Y
atcggaatcattaggcctggtggtagcacatactacgcgagctgggcgaaaggccgattc
  I G I I R P G G S T Y Y A S W A K G R F
accatctccaaaacctcgtcgaccacggtggatctgaaaatgaccagtctgacaaccgag
  T I S K T S S T T V D L K M T S L T T E
gacacggccacctatttctgtgccagagcctgggacatctggggcccaggcaccctggtc
  D T A T Y F C A R A W D I W G P G T L V
accgtctccttagggcaacctaaggctccatcagtcttcccactggccccctgctgcggg
  T V S L G Q P K A P S V F P L A P C C G
gacacacccagctccacggtgaccctgggctgcctggtcaaaggctacctcccggagcca
  D T P S S T V T L G C L V K G Y L P E P
gtgaccgtgacctggaactcgggcaccctcaccaatggggtacgcaccttcccgtccgtc
  V T V T W N S G T L T N G V R T F P S V
cggcagtcctcaggcctctactcgctgagcagcgtggtgagcgtgacctcaagcagccag
  R Q S S G L Y S L S S V V S V T S S S Q
cccgtcacctgcaacgtggcccacccagccaccaacaccaaagtggacaagaccgttgcg
  P V T C N V A H P A T N T K V D K T V A
ccctcgacatgcagcaagcccacgtgcccaccccctgaactcctgggcggccgccctcaa
  P S T C S K P T C P P P E L L G G R P Q
ccccaaccacaaccgcaacctcagccccagccacagccgcagccgcagccacaaccccag
  P Q P Q P Q P Q P Q P Q P Q P Q P Q P Q
cctcaaccacaaccccagcctcaacctcag [symbol] cagtcggtggaggagtccgggggt
  P Q P Q P Q P Q P Q S R Q S V E E S G G
cgcctggtcacgcctgggacacccctgacactcacctgcacagtctctggattctccctc
  R L V T P G T P L T L T C T V S G F S L
aataccaatgcaataaggtgggtccgccaggctccagggaaggggctggaatggatcgga
  N T N A I R W V R Q A P G K G L E W I G
gccattgatgctagtggtaacacatactacgcgagctgggcgaaaggccgattcaccatc
  A I D A S G N T Y Y A S W A K G R F T I
tccaaaacctcgaccacggtggatctgaaaatcaccagtccgacaaccgaggacacggcc
  S K T S T T V D L K I T S P T T E D T A
acctatttctgtaacagatttaatactgccaacatctggggcccaggcaccctggtcacc
  T Y F C N R E N T A N I W G P G T L V T
gtctcctcagggcaacctaaggctccatcagtcttcccactggccccctgctgcggggac
  V S S G Q P K A P S V F P L A P C C G D
acacccagctccacggtgaccctgggctgcctggtcaaaggctacctcccggagccagtg
  T P S S T V T L G C L V K G Y L P E P V
accgtgacctggaactcgggcaccctcaccaatggggtacgcaccttcccgtccgtccgg
  T V T W N S G T L T N G V R T F P S V R
cagtcctcaggcctctactcgctgagcagcgtggtgagcgtgacctcaagcagccagccc
  Q S S G L Y S L S S V V S V T S S S Q P
gtcacctgcaacgtggcccacccagccaccaacaccaaagtggacaagaccgttgcgccc
  V T C N V A H P A T N T K V D K T V A P
tcgacatgcagcaagcccacgtgcccaccccctgaactcctggggggaccgtctgtcttc
  S T C S K P T C P P P E L L G G P S V F
atcttccccccaaaacccaaggacaccctcatgatctcacgcacccccgaggtcacatgc
  I F P P K P K D T L M I S R T P E V T C
gtggtggtggacgtgagccaggatgaccccgaggtgcagttcacatggtacataaacaac
  V V V D V S Q D D P E V Q F T W Y I N N
gagcaggtgcgcaccgcccggccgccgctacgggagcagcagttcaacagcacgatccgc
  E Q V R T A R P P L R E Q Q F N S T I R
gtggtcagcaccctccccatcgcgcaccaggactggctgaggggcaaggagttcaagtgc
  V V S T L P I A H Q D W L R G K E F K C
aaagtccacaacaaggcactcccggcccccatcgagaaaaccatctccaaagccagaggg
  K V H N K A L P A P I E K T I S K A R G
cagcccctggagccgaaggtctacaccatgggccctccccgggaggagctgagcagcagg
  Q P L E P K V Y T M G P P R E E L S S R
tcggtcagcctggcctgcatgatcaacggcttctacccttccgacatctcggtggagtgg
  S V S L A C M I N G F Y P S D I S V E W
gagaagaacgggaaggcagaggacaactacaagaccacgccggccgtgctggacagcgac
  E K N G K A E D N Y K T T P A V L D S D
ggctcctacttcctctacagcaagctctcagtgcccacgagtgagtggcagcggggcgac
  G S F L Y Y S K L S V P T S E W Q R G D
gtcttcacctgctccgtgatgcacgaggccttgcacaaccactacacgcagaagtccatc
  V F T C S V M H E A L H N H Y T Q K S I
tcccgctctccgggtaaatgaggatcc
  S R S P G K -
```

Heavy Chain Construct 5 (SEQ ID NOS 31-32, respectively, in order of appearance) contained one heavy sequence from Antibody 3 linked and in-frame with a second heavy chain sequence, as set forth below. Both heavy chain sequences were developed from a single chain Antibody 3. Bold and underlined DNA sequences are restriction sites used for subcloning to build the constructs. The protein sequence in bold italics is the signal peptide. The underlined sequences are the two variable domains in tandem of Antibody 3. The PQ Linker is highlighted. The His-tag near the C-terminus of the protein is in

[symbol]

```
gctagcaggactgagcacagacgactcaccatggagctggggctgagctgggtggtcctg

[symbol]

gctgctctactacaaggtgtccaggctgaggtgcagctggtggagtctgggggaggcttg [symbol]
  V Q L V E S G G G L
gtgcagcctggggggtctctgaggctctcctgtgtagcctctggaaacatgttcggcatt
  V Q P G G S L R L S C V A S G N M F G I
aacacgatgggctggttccgcgcggctccagggaaagagcgtcagtttgtagcagctgat
  N T M G W F R A A P G K E R Q F V A A D
aatttcagtggtggcaggaagaatattgacttctccgtgaggggtcgattcaccatgtca
  N F S G G R K N I D F S V R G R F T M S
agagacgccgccagcaacacgatatatctacaaatgaacagcctgaaagttgaggacacg
  R D A A S N T I Y L Q M N S L K V E D T
gccgtttattactgcgcagcgggtgcgaatacgatgatgattgtggcggacaattacgaa
  A V Y Y C A A G A N T M M I V A D N Y E
tactggggtcaggggacccaggtcaccgtctcctcagaacccaagacaccaaaaagcggc
  Y W G Q G T Q V T V S S E P K T P K S G
cgccctcaaccccaaccacaaccgcaacctcagccccagccacagccgcagccgcagcca
  R P Q P Q P Q P Q P Q P Q P Q P Q P Q P
caaccccagcctcaaccacaaccccagcctcaacctcagtctagagctcaggtgcagctg
  Q P Q P Q P Q P Q P Q P Q S R A Q V Q L
gtggagtctgggggaggcttggtgcagcctggggggtctctgaggctctcctgtgtagcc
  V E S G G G L V Q P G G S L R L S C V A
tctggaaacatgttcggcattaacacgatgggctggttccgcgcggctccagggaaagag
  S G N M E G I N T M G W E R A A P G K E
cgtcagtttgtagcagctgataatttcagtggtggcaggaagaatattgacttctccgtg
  R Q F V A A D N F S G G R K N I D F S V
aggggtcgattcaccatgtcaagagacgccgccagcaacacgatatatctacaaatgaac
  R G R F T M S R D A A S N T I Y L Q M N
agcctgaaagttgaggacacggccgtttattactgcgcagcgggtgcgaatacgatgatg
  S L K V E D T A V Y Y C A A G A N T M M
attgtggcggacaattacgaatactggggtcaggggacccaggtcaccgtctcctcagaa
  I V A D N Y E Y W G Q G T Q V T V S S E
cccaagacaccaaaaaagcttccacagcctcagccgcagccccagccacaaccacaaccc
  P K T P K K L P Q P Q P Q P Q P Q P Q P
aatcctacaacagaatccaagtgtcccaaatgtccagcccctgagctcctgggagggccc
  N P T T E S K C P K C P A P E L L G G P
tcagtcttcatcttccccccgaaacccaaggacgtcctctccatttctgggaggcccgag
  S V F I F P P K P K D V L S I S G R P E
gtcacgtgcgttgtggtagacgtgggccaggaagaccccgaggtcagtttcaactggtac
  V T C V V V D V G Q E D P E V S F N W Y
attgatggcgctgaggtgcgaacggccaacacgaggccaaaagaggaacagttcaacagc
  I D G A E V R T A N T R P K E E Q F N S
acgtaccgcgtggtcagcgtcctgcccatccagcaccaggactggctgacggggaaggaa
  T Y R V V S V L P I Q H Q D W L T G K E
ttcaagtgcaaggtcaacaacaaagctctcccggcccccatcgagaagaccatctccaag
  F K C K V N N K A L P A P I E K T I S K
gccaaagggcagacccgggagccgcaggtgtacgccctggccccacaccgggaagagctg
  A K G Q T R E P Q V Y A L A P H R E E L
gccaaggacaccgtgagcgtaacctgcctggtcaaaggcttctacccacctgatatcaac
  A K D T V S V T C L V K G E Y P P D I N
gttgagtggcagaggaacggtcagccggagtcagagggcacctacgccaccacgccaccc
  V E W Q R N G Q P E S E G T Y A T T P P
cagctggacaacgacgggacctacttcctctacagcaagctctcggtgggaaagaacacg
  Q L D N D G T Y F L Y S K L S V G K N T
tggcagcggggagaaaccttcacctgtgtggtgatgcatgaggccctgggatccagcacc
  W Q R G E T F T C V V M H E A L G S S T
ggtcaccatcaccaccatcaccaccacgggtga
  G [symbol]   G -
```

### Example 2: Generation of Light Chain Constructs for 2-Chain Antibodies

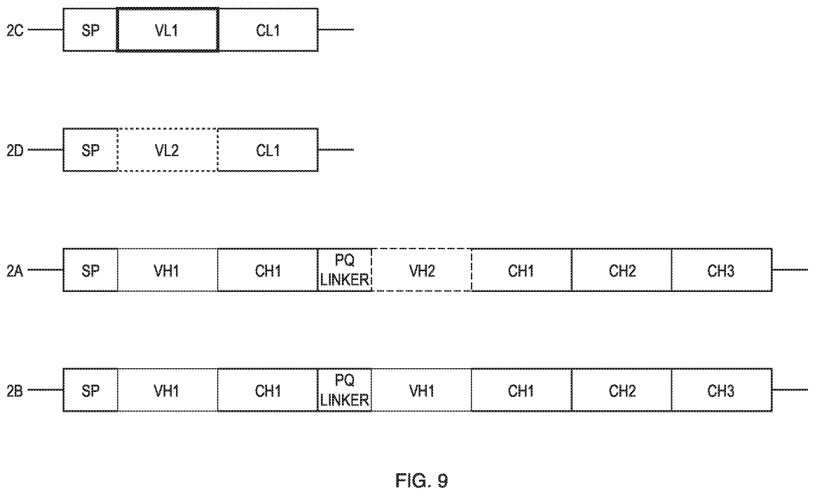

DNA plasmids corresponding to the Light Chains were constructed in order to create the following two functional 2-chain rabbit antibodies (FIG. 9). The Light Chain constructs consisted of the natural signal peptide present on light chains and contained the full length light chain sequence.

Light Chain Construct 1 (SEQ ID NOS 37-38, respectively, in order of appearance) was derived from Antibody 1. The translated protein comprising the full length Antibody 1 is underlined.

```
atggacacgagggcccccactcagctgctggggctcctgctgctctggctcccaggtgcc
  M D T R A P T Q L L G L L L L W L P G A
acatttgcgcaagtgctgacccagactgcatcgcccgtgtctgcagctgtgggaggcaca
  T F A Q V L T Q T A S P V S A A V G G T
gtcaccatcagttgccagtccagtcagagtgttyataataccaataacttatcctggtat
  V T I S C Q S S Q S V X N T N N L S W Y
cagcagaaaccarggcagcctcccaagcgcctgatctattctgcatccactctggaatct
  Q Q K P X Q P P K R L I Y S A S T L E S
ggggtcccatcgcggttcaaaggcagtggatctgggacacagttcactctcaccatcagc
  G V P S R F K G S G S G T Q F T L T I S
gacctggagtgtgacgatgctgccacttactactgtgcaggcggttatagtgatattaat
  D L E C D D A A T Y Y C A G G Y S D I N
gctttcggcggagggaccgaggtggtcgtcaaaggtgatccagttgcacctactgtcctc
  A F G G G T E V V V K G D P V A P T V L
atcttcccaccatctgctgatcttgtggcaactggaacagtcaccatcgtgtgtgtggcg
  I F P P S A D L V A T G T V T I V C V A
aataaatactttcccgatgtcaccgtcacctgggaggtggatggcaccacccaaacaact
  N K Y F P D V T V T W E V D G T T Q T T
ggcatcgagaacagtaaaacaccgcagaattctgcagattgtacctacaacctcagcagc
  G I E N S K T P Q N S A D C T Y N L S S
actctgacactgaccagcacacagtacaacagccacaaagagtacacctgcaaggtgacc
  T L T L T S T Q Y N S H K E Y T C K V T
cagggcacgacctcagtcgtccagagcttcaataggggtgactgttag
  Q G T T S V V Q S F N R G D C -
```

Light Chain Construct 2 (SEQ ID NOS 39-40, respectively, in order of appearance) was derived from Antibody 2. The translated protein comprising the full length Antibody 2 is underlined.

```
atggacacgagggcccccactcagctgctgggactcctgctgctctggctcccaggtgcc
  M D T R A P T Q L L G L L L L W L P G A
agatgtgcattcgaattgacccagactccatcctccgtggaggcagctgtgggaggcaca
  R C A F E L T Q T P S S V E A A V G G T
gtcaccatcaggtgccaggccagtcagagcattagcaaccaactatcctggtatcagcag
  V T I R C Q A S Q S I S N Q L S W Y Q Q
aaaccagggcagcctcccaagcgcctgatctacagggcatccactctggcatctggggtc
  K P G Q P P K R L I Y R A S T L A S G V
ccagcgcgtttcaaaggcagtggatctggtacagcgttcactctcaccatcagcgacctg
  P A R F K G S G S G T A F T L T I S D L
gagtgtgccgatggtgccacttactactgtcaaagtttttattatactgatgttattgat
  E C A D G A T Y Y C Q S F Y Y T D V I D
tataatgctttcggcggagggaccgaggtggtggtcacaggtgatccagttgcacctact
  Y N A F G G G T E V V V T G D P V A P T
gtcctcatcttcccaccagctgctgatcaggtggcaactggaacagtcaccatcgtgtgt
  V L I F P P A A D Q V A T G T V T I V C
gtggcgaataaatactttcccgatgtcaccgtcacctgggaggtggatggcaccacccaa
  V A N K Y F P D V T V T W E V D G T T Q
acaactggcatcgagaacagtaaaacaccgcagaattctgcagattgtacctacaacctc
  T T G I E N S K T P Q N S A D C T Y N L
agcagcactctgacactgaccagcacacagtacaacagccacaaagagtacacctgcaag
  S S T L T L T S T Q Y N S H K E Y T C K
gtgacccagggcacgacctcagtcgtccagagcttcaataggggtgactgttag
  V T Q G T T S V V Q S F N R G D C -
```

### Example 3: Expression in Mammalian Expression Vector

A number of transfections were completed and the secreted antibodies were purified and tested in a variety of assays as described in Example 4. The following constructs were transfected or co-transfected into HEK293 cells using 293 Fectin:

a) Heavy Chain Construct 1 with Light Chain Construct 1 (VH1−VH1/VL1)
b) Heavy Chain Construct 2 with Light Chain Construct 2 (VH2−VH2/VL2)
c) Heavy Chain Construct 3 with Light Chain Construct 1 (VH1−VH2/VL1)
d) Heavy Chain Construct 3 with Light Chain Construct 2 (VH1−VH2/VL2)
e) Heavy Chain Construct 3 with Light Chain Constructs 1+2 (VH1−VH2/VL1+VL2)
f) Heavy Chain Construct 4 with Light Chain Construct 1 (VH2−VH1/VL1)
g) Heavy Chain Construct 4 with Light Chain Construct 2 (VH2−VH1/VL2)
h) Heavy Chain Construct 4 with Light Chain Constructs 1+2 (VH2−VH1/VL1+VL2)
i) Original Heavy Chain (VH1) and Light Chain (VL1) that recognizes Antigen 1.
j) Original Heavy Chain (VH2) and Light Chain (VL2) that recognizes Antigen 2.
k) Heavy Chain Construct 5 with 2 VHH domains against Rabbit IgG
l) Original Heavy Chain Construct of 1 VHH domain against Rabbit IgG

Supernatants from these samples were collected and the secreted antibodies present within these supernatants were tested by enzyme-linked immunosorbent assay (ELISA) and Immunohistochemistry (IHC), or purified using Protein A (for 2-chain antibodies with Fc) or

Nickel (for His-tagged single chain antibodies).

### Example 4: Functional Testing of Antibodies Using ELISA and/or IHC

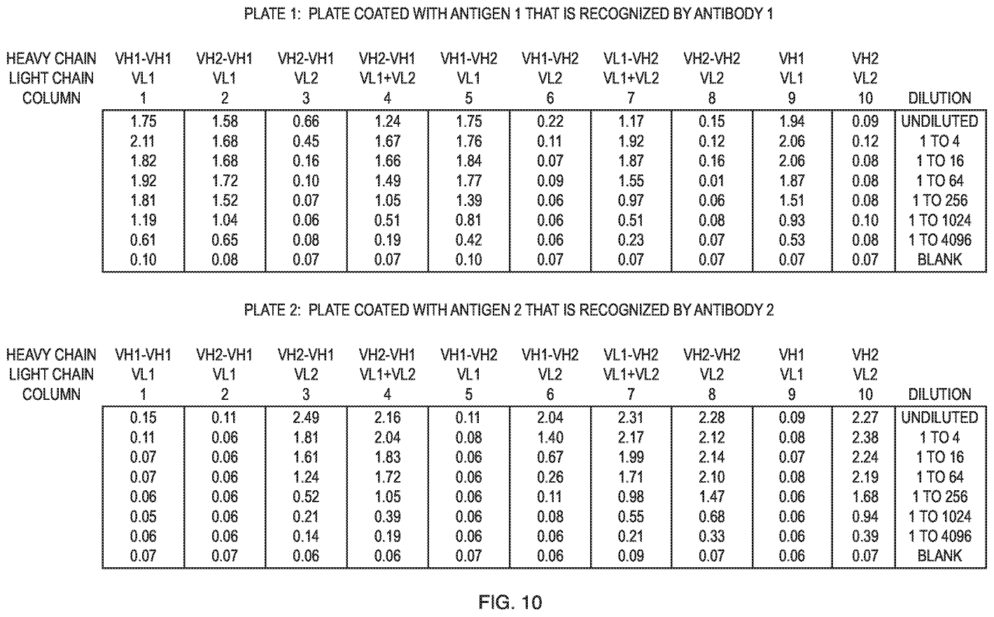

Two-chain secreted antibodies were tested for specificity by ELISA (See FIG. 10). ELISA testing was performed with standard procedures using the supernatants obtained from transfections listed above (a-j). Serially diluted (1 to 4) samples were tested with both Antigen 1 and Antigen 2 to test for specificity. The original VH1/VL1 rabbit monoclonal is specific for Antigen 1 (compare Plate 1: Column 9 with Plate 2: Column 9). VH2/VL2 rabbit monoclonal is specific for Antigen 2 (compare Plate 2: Column 10 with Plate 1: Column 10). As summarized in Table 1, the activity of the each antibody was retained when the heavy chain and corresponding light chain were present in the transfection.

TABLE 1

| Heavy Chain | Light Chain | Antigen 1 | Antigen 2 |
|---|---|---|---|
| VH1-VH1 | VL1 | + | − |
| VH2-VH1 | VL1 | + | − |
| VH2-VH1 | VL2 | − | + |
| VH2-VH1 | VL1 + VL2 | + | + |
| VH1-VH2 | VL1 | + | − |
| VH1-VH2 | VL2 | − | + |
| VH1-VH2 | VL1 + VL2 | + | + |
| VH1 only | VL1 | + | − |
| VH2 only | VH2 | − | + |

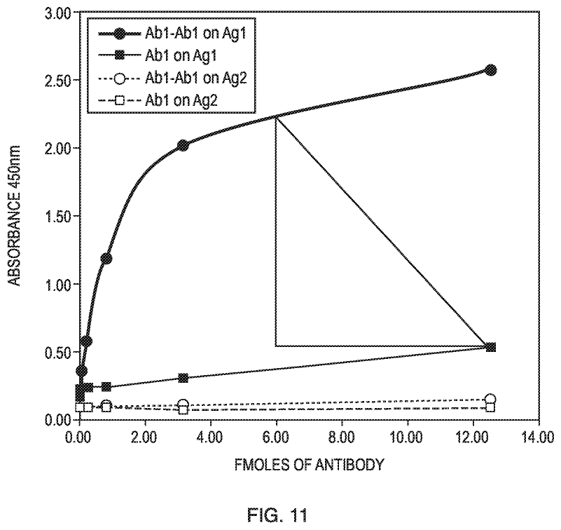

The experiments corresponding to FIG. 10 did not take into account relative amounts (in terms of moles) of the antibodies. However the experiments corresponding to FIG. 11 illustrate the sensitivity and specificity of the 2-chain secreted antibodies by ELISA. ELISA testing was performed with standard procedures. As above, the stacked sample (Ab1-Abl) and the original rabbit monoclonal (Ab1) was tested against Ag1 (positive control antigen) and Ag2 (negative control antigen) to test for specificity. Both the original and stacked 2-chain antibodies were specific for Ag1 and did not recognize Ag2. As can be seen from FIG. 11, the binding of the stacked rabbit monoclonal was much greater than twice the binding of the original, single copy, monoclonal as depicted by the right triangle in FIG. 11. This shows that the activity of the secreted stacked 2-chain antibody was not additive, but instead synergistic when compared to the original 2-chain rabbit monoclonal.

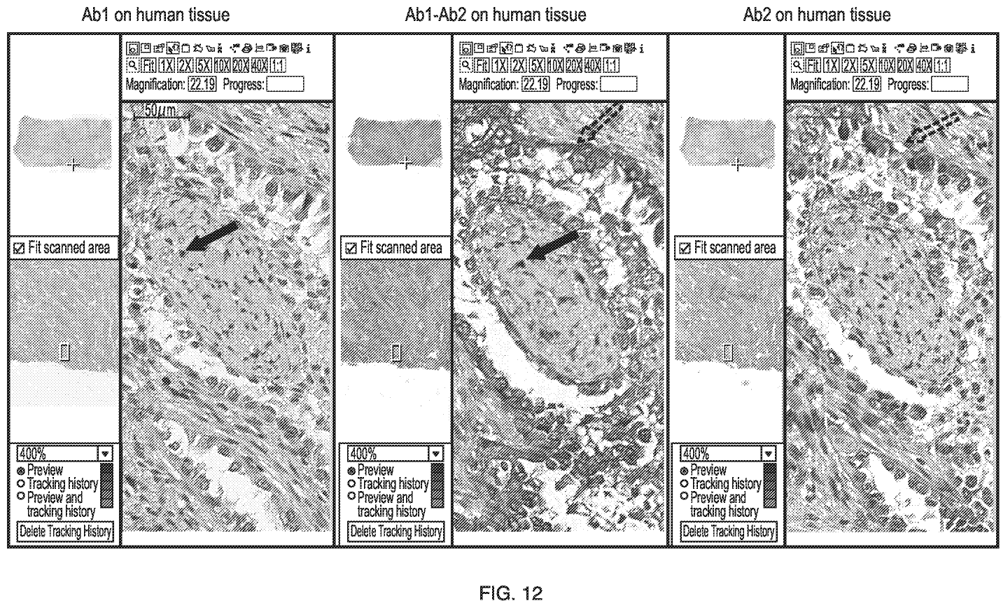

With respect to specificity, FIG. 12 illustrates that in the 2 chain stacked conformation (whereby 2 different antibodies were covalently bound (Ab1-Ab2)), both antibodies recognized their targets. The solid black arrow points to cells that Ab1 recognizes. The dashed arrow points to membranes that are stained by Ab2. In the stacked antibody, both staining patterns are visible.

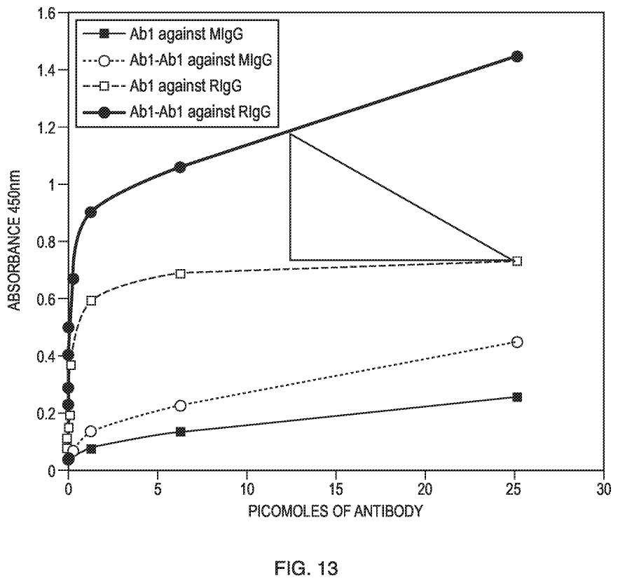

Single chain secreted antibodies were tested by ELISA (see FIG. 13). ELISA testing was performed with standard procedures using the supernatants obtained from transfections listed above (k-1). Serially diluted (1 to 4) samples were tested against Rabbit IgG (which was used to generate the original monoclonal) and Mouse IgG (as a negative control) to test for specificity. The original single-chain VH1 llama monoclonal was specific for Rabbit IgG (compare black lines). The stacked single-chain VH1-VH1 antibody was specific for Rabbit IgG as well (compare gray lines). Again, as shown in FIG. 13, the binding of the stacked llama monoclonal (dotted gray line) was much greater than twice the binding of a single copy monoclonal (dotted black line) as depicted by the right triangle in FIG. 13. This result showed that the activity of the secreted, stacked single chain antibody was not additive, but rather synergistic when compared to the original single chain antibody.

### Example 5: Monovalent and Divalent Antibody Binding

Three rabbit monoclonal antibodies were identified that exhibited specificity to certain peptide sequences. These three rabbit monoclonal antibodies (VAR₁, VAR₂, and VAR₃) were typical in that the heavy chains were encoded by a single variable domain sequence. The heavy chains of these three antibodies were engineered so that each would contain two of the same variable domain (VAR₁-VAR₁, VAR₂-VAR₂, VAR₃-VAR₃), similar to the constructs of Heavy Chain Construct 1 and Heavy Chain Construct 2 (discussed above).

It was hypothesized that modifying these antibodies so that they contain four binding domains instead of the two present in the original antibodies would result in a synergistic effect on binding affinity/avidity. Therefore, the binding of these modified antibodies would be greater than twice the binding measurement of the original antibody. This hypothesize was confirmed by the experiments described herein. Specifically, as discussed in further detail below, in the worst case, the binding of the modified antibody was 10 times stronger than the original antibody. In the best case, the binding of the modified antibody was approximately 440 times stronger than the original antibody.

Initially, a scouting assay was performed on all three of the original rabbit monoclonal antibodies and the modified versions versus their peptide ligands. Subsequently, full kinetics analyses were performed on two of the three sets.

Specifically, affinity/Avidity measurements of six divalent rabbit monoclonal antibodies against three peptides were conducted to determine if the modifications resulted in increased binding to target peptides. Affinity/avidity measurements were determined using by surface plasmon resonance. Biacore is a surface plasmon resonance machine that performs real time measurement of molecular interaction by changes in mass-induced refractory index. Ligand is captured on a chip surface backed by colloidal gold membrane. Refractory index of polarized light shifts when change in molecular mass occurs due to analyte binding or dissociation from captured ligand. Changes in this refractory index are captured as a sensorgram and a real time measurement of association constant and dissociation constant is calculated by Biacore curve fitting program. Equilibrium binding constant (K_{D}) is calculated from the ratio of dissociation constant (kd) and association constant (ka). With the SPR analysis, information such as specificity, affinity (how strong is the binding), kinetics (how fast are the kinetics), and activity (if the ligand immobilized s 100% active) can be captured.

The following abbreviations are used in this Example.

TABLE 2
Abbreviations

| | |
|---|---|
| SA chip | Streptavidin Coated Chip |
| CM5 | Carboxy methylated dextran chip |
| Chip | |
| SPR | Surface Plasmon Resonance |
| RU | Response Units |
| R_{L} | Ligand RU |
| K_{D} | Equilibrium binding affinity constant. |
| | K_{D} is also defined as the analyte concentration at which 50% of |
| | the maximum available ligand is in complex form at equilibrium |
| Analyte | One of the interacting molecule flown over the surface. In these |
| | experiments, the antibody is the analyte that binds to the peptide |
| | (ligand). |
| Ligand | One of the interacting molecule immobilized or captured on the |
| | surface. In these experiments, the peptide is the ligand to which |
| | the antibody (analyte) binds to. |
| Rmax | Maximum binding capacity (in RU) of ligand |
| | captured/immobilized on the surface |
| MW | Molecular weight |
| HBS- | HEPES buffered saline supplemented with EDTA and P20 |
| EP | |
| ka | Association rate constant in M⁻¹s⁻¹ |
| kd | Dissociation rate constant in s⁻¹ |
| χ² | Chi squared |

A. Exemplary Antibodies and Peptides

VAR₁ Antibody is a divalent monoclonal antibody encoded by a heavy chain cDNA with a single variable domain and a light chain cDNA with a single variable domain. Since this antibody is divalent, there are two binding domains per antibody molecule. VAR₁ antibody is specific for Peptide₁.

VAR₁-VAR₁ Antibody is a divalent monoclonal antibody encoded by a heavy chain cDNA with two variable domains in tandem and a light chain cDNA with a single variable domain. Since this antibody is divalent, there are four binding domains per antibody molecule. VAR₁-VAR₁ antibody is specific for Peptide₁.

VAR₂ Antibody is a divalent monoclonal antibody encoded by a heavy chain cDNA with a single variable domain and a light chain cDNA with a single variable domain. Since this antibody is divalent, there are two binding domains per antibody molecule. VAR₂ antibody is specific for Peptide₂.

VAR₂-VAR₂ Antibody is a divalent monoclonal antibody encoded by a heavy chain cDNA with two variable domains in tandem and a light chain cDNA with a single variable domain. Since this antibody is divalent, there are four binding domains per antibody molecule. VAR₂-VAR₂ antibody is specific for Peptide₂.

VAR₃ Antibody is a divalent monoclonal antibody encoded by a heavy chain cDNA with a single variable domain and a light chain cDNA with a single variable domain. Since this antibody is divalent, there are two binding domains per antibody molecule. VAR₃ antibody is specific for Peptide₃.

VAR₃-VAR₃ Antibody is a divalent monoclonal antibody encoded by a heavy chain cDNA with two variable domains in tandem and a light chain cDNA with a single variable domain. Since this antibody is divalent, there are four binding domains per antibody molecule. VAR₃-VAR₃ antibody is specific for Peptide₃.

B. Binding Experiments

The Biacore 3000 was used. Antibodies were used as analytes and peptides were used as ligands. The analytes and ligands used are set forth below in Table 3 and 4, respectively

TABLE 3
Analytes

| | Analyte | | | | | |
|---|---|---|---|---|---|---|
| | 1 | 2 | 3 | 4 | 5 | 6 |
| Sample ID | VAR₁ | VAR₁ - | VAR₂ | VAR₂- | VAR₃ | VAR₃- |
| | Antibody | VAR₁ | Antibody | Var₂ | Antibody | Var₃ |
| | | Antibody | | Antibody | | Antibody |
| MW | 150 | 250 | 150 | 250 | 150 | 250 |
| Species | Rabbit | Rabbit | Rabbit | Rabbit | Rabbit | Rabbit |
| Buffer composition | PBS + | PBS + | PBS + | PBS + | PBS + | PBS + |
| | 0.05% | 0.05% | 0.05% | 0.05% | 0.05% | 0.05% |
| | Azide | azide | azide | azide | azide | azide |
| Concentration | 0.3 | 0.3 | 0.2 | 0.3 | 0.5 mg/ml | 1 mg/ml |
| | mg/ml | mg/ml | mg/ml | mg/ml | | |
| Any Tags | None | None | None | None | None | None |
| Purity | Purified | Purified | Purified | Purified | Purified | Purified |
| | mAb | mAb | mAb | mAb | mAb | mAb |
| Storage temperature | 4c | 4c | 4c | 4c | 4c | 4c |

TABLE 4
Ligands

| | Ligand Immobilized/Captured on Surface | | |
|---|---|---|---|
| | 1 | 2 | 3 |
| Sample ID | Peptide₁ | Peptide₂ | Peptide₃ |
| MW | 1.244 kDa | 1.68 kDa | 2.36 kDa |
| Species | | | |
| Buffer composition | DMSO | DMSO | DMSO |
| Concentration | 20 mg/ml | 20 mg/ml | 20 mg/ml |
| Any Tags | Biotinylated | Biotinylated | Biotinylated |
| Purity | 70% | 70% | 70% |
| Storage Temp. | −20 c. | −20 c. | −20 c. |

Binding experiments were performed on Biacore 3000 at 25° C. The sensor chip was a certified grade SA chip. The assay buffer was 10 mM HEPES buffer (pH 7.4), 150 mM NaCl, 3 mM EDTA, 0.05% P20 (polyoxoethylenesorbitan). The regeneration or surface activation buffer was 10 mM Glycine HCl Ph 1.75 for CM5 chip and 1M NaCl/50 mM NaOH for SA chip. Biocytin was used at 10 ug/ml. All the assays were performed at room temperature. The sensor chip and running buffer were equilibrated to room temperature.

Flow cells 2 and 3 of the SA chip were coated captured by biotinylated peptide at an RU as indicated. The flow cell 1 was untreated and was used as blank for reference subtraction. The unoccupied sites were blocked with biocytin. The analyte was flowed over the chip at a single concentration. Binding of analyte to the ligand was monitored in real time.

The amount (R_{L}) of protein to be captured on the chip depends on the ligand and analyte molecular weight and were determined by the following formula: R_{Max}=MW_{A}/MW_{L} ·R_{L} MW_{A} is the molecular weight of the analyte. MW_{L} is the molecular weight of the ligand

K_{D} was defined by the following equation: Biacore measures real time ka (on rate) and kd (off rate) based on their curve fitting program.

K_{D}, ka and kd were measured using BIA evaluation software with Langmuir 1:1 curve fitting model.

Chi square (χ²) analysis on the observed and the fitted curve was performed to determine the accuracy of the fitted curve compared to the measured curve. χ² value of ˜2 was considered significant (accurate) and below 1 was highly significant (highly accurate). For the interaction with fast off rate, steady state kinetic analysis was performed to determine KD.

C. Scouting Analysis

For determining the working conditions, a simple scouting analysis was performed at single analyte concentration. Scouting results determine Yes/No binding and the analyte concentration range to be used for full kinetic analysis. After each run and or assay, the surface was regenerated to remove any bound analyte and equilibrated with running buffer until a stable baseline was established.

The results of the scouting analysis are set forth below.

TABLE 5
VAR₁ Antibody

| | | | | | | | |
|---|---|---|---|---|---|---|---|
| | | | | Rmax | Conc. Of | | |
| Ligand | Analyte | ka(1/Ms) | kd(1/s) | (RU) | Analyte | KD(M) | Chi² |

| | | | | | | |
|---|---|---|---|---|---|---|
| Peptide₁ | 1.57 × 10⁷ | 2.90 × 10⁻³ | 140 | 10 nM | 1.85 × 10⁻¹⁰ | 0.436 |
| 350 RU | | | | | | |

TABLE 6
VAR₁ -VAR₁ Antibody

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| Peptide₁ | VAR₁ - | 1.13 × 10⁶ | 2.87 × 10⁻⁵ | 1300 | 10 nM | 2.53 × 10⁻¹¹ | 7.88 |
| 350 RU | VAR₁ | | | | | | |
| | Antibody | | | | | | |

TABLE 7
VAR₂ Antibody

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| Peptide₂ | VAR₂ | 2.44 × 10⁶ | 4.24 × 10⁻⁵ | 166 | 10 nM | 1.73 × 10⁻¹¹ | 0.0347 |
| 15 RU | | | | | | | |

TABLE 8
VAR₂ -VAR₂ Antibody

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| Peptide₂ | VAR₂- | 5.57 × 10⁶ | 1.18 × 10⁻⁶ | 341 | 10 nM | 2.13 × 10⁻¹³ | 29.8 |
| 15 RU | VAR₂ | | | | | | |
| | Antibody | | | | | | |

TABLE 9
VAR₃ Antibody

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| Peptide₃ | VAR₃ | 1.23 × 10⁶ | 3.68 × 10⁻⁵ | 115 | 10 nM | 2.99 × 10⁻¹¹ | 0.0136 |
| 15 RU | | | | | | | |

TABLE 10
VAR₃ - VAR₃ Antibody

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| Peptide₃ | VAR₃₋ | 1.94 × 10⁶ | 1.10 × 10⁻⁶ | 127 | 10 nM | 5.68 × 10⁻¹³ | 0.339 |
| 15 RU | VAR₃ | | | | | | |
| | Antibody | | | | | | |

From this data, the comparisons of binding affinity/avidity were calculated between the original antibodies (VAR₁, VAR₂, and VAR₃) and their modified counterparts (VAR₁-VAR₁, VAR₂-VAR₂, VAR₃-VAR₃), as shown in Table 11.

TABLE 11
Comparisons

| | | | | Fold |
|---|---|---|---|---|
| | | | VAR_{N}- VAR_{N} | Increase |
| | | | Antibody K_{D} | in binding |
| | VAR_{N} | VAR_{N}-VAR_{N} | V̅A̅R̅_̅{̅N̅}̅ A̅n̅t̅i̅b̅o̅d̅y̅ | affinity/ |
| Peptide | Antibody K_{D} | Antibody K_{D} | K_{D} | avidity |
| Peptide₁ | 1.85 × 10⁻¹⁰ | 2.53 × 10⁻¹¹ | 0.137 | 7.3-fold |
| Peptide₂ | 1.73 × 10⁻¹¹ | 2.13 × 10⁻¹³ | 0.012 | 83.3-fold |
| Peptide₃ | 2.99 × 10⁻¹¹ | 5.68 × 10⁻¹³ | 0.019 | 52.6-fold |

Increases in binding affinity and avidity were observed in all cases when the divalent antibodies containing four sets of binding domains (encoded by VAR_{N}-VAR_{N} heavy chain constructs) were compared with their original versions containing only two sets of binding domains (encoded by VAR_{N} heavy chain constructs). If the modification of doubling binding domains (from 2 to 4) were additive, then the results were considered doubling of affinity. If the modifications were synergistic, then the result of doubling binding domains were affinity increases of greater than 2-fold.

D. Full Kinetics

Since the scouting analysis was performed at a single analyte concentration and provides a rough estimate, full kinetic analysis was performed to confirm the scouting analysis and provides a more complete data set. Therefore, antibody sets that were specific for Peptide 2 and 3 were further evaluated by full kinetic analysis.

Full kinetics was performed at a range of analyte concentrations with two fold serial dilution in the assay buffer. Analyte was flowed over the surface of ligand, starting from lowest 0 nM concentration to the highest.

Chi square (χ²) analysis was carried out between the actual Sensorgram (colored line) and the sensorgram generated from the BlAnalysis software (black line) to determine the accuracy of the analysis.

χ² value within 1-2 was considered significant (accurate) and below 1 was highly significant (highly accurate).

The results of the full kinetics analysis are set forth below in Tables 12-15.

TABLE 12
Full Kinetics - VAR₂ Antibody

| | | | | Rmax | Conc. of | | |
|---|---|---|---|---|---|---|---|
| Ligand | Analyte | ka(1/Ms) | kd(1/s) | (RU) | Analyte | KD(M) | Chi² |
| Peptide₂ | VAR₂ Antibody | 8.89 × 10⁵ | 3.11 × 10⁻⁴ | 126 | 0-5 nM | 3.49 × 10⁻¹⁰ | 0.0638 |
| 15 RU | | | | | | | |

TABLE 13
Full Kinetics - VAR₂ - VAR₂ Antibody

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| Peptide₂ | VAR₂- | 7.16 × 10⁶ | 5.53 × 10⁻⁶ | 171 | 0-2.5 nM | 7.73 × 10⁻¹³ | 0.649 |
| 15 RU | VAR₂ | | | | | | |
| | Antibody | | | | | | |

TABLE 14
Full Kinetics - VAR₃ Antibody

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| Peptide₃ | VAR₃ | 1.35 × 10⁶ | 3.67 × 10⁻⁵ | 94.5 | 0-10 nM | 2.72 × 10⁻¹¹ | 0.655 |
| 15 RU | Antibody | | | | | | |

TABLE 15
Full Kinetics - VAR₃ - VAR₃ Antibody

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| Peptide₃ | VAR₃- | 2.04 × 10⁶ | 2.85 × 10⁻⁶ | 108 | 0-10 nM | 1.39 × 10⁻¹² | 1.6 |
| 15 RU | VAR₃ | | | | | | |
| | Antibody | | | | | | |

From the full kinetics data set, the comparisons of binding affinity/avidity were calculated between the original antibodies (VAR₂ and VAR₃) and their modified counterparts (VAR₂-VAR₂ and VAR₃-VAR₃), as shown in Table 16.

TABLE 16
Comparison

| | | | | Fold |
|---|---|---|---|---|
| | | | VAR_{N}-VAR_{N} | Increase |
| | | | Antibody K_{D} | in binding |
| | VAR_{N} | VAR_{N}-VAR_{N} | V̅A̅R̅_̅{̅N̅}̅ A̅n̅t̅i̅b̅o̅d̅y̅ | affinity/ |
| Peptide | Antibody K_{D} | Antibody K_{D} | K_{D} | avidity |
| Peptide₂ | 3.49 × 10⁻¹⁰ | 7.73 × 10⁻¹³ | 0.0022 | 454.5-fold |
| Peptide₃ | 2.72 × 10⁻¹¹ | 1.39 × 10⁻¹² | 0.0511 | 19.6-fold |

This data reveals that increasing the binding domains of these antibodies by a factor of two results in binding affinity/avidity increases of greater than a factor of two. With respect to the VAR₂ antibody compared with the VAR₂-VAR₂ modified version of the antibody, the increase is 454 fold. With respect to the VAR₃ antibody compared to its modified version VAR₃-VAR₃ antibody, the increase is almost 20 fold. This data clearly demonstrates that the modification of increasing the binding domains as described herein is not merely additive, but instead has a synergistic effect on affinity/avidity.

SUMMARY OF SEQUENCE LISTING

SEQ ID NO: 1
Exemplary Leader Sequence for Rabbit Antibody (nucleotide)
atggagactgggctgcgctggcttctcctggtcgctgtgctcaaaggtgtccagtgt
SEQ ID NO: 2
Exemplary Leader Sequence for Rabbit Antibody (amino acid)
M E T G L R W L L L V A V L K G V Q C
SEQ ID NO: 3
Exemplary Leader Sequence for Llama Single Chain Antibody (nucleotide)
Atggagaggggctgagagggtggtcctggctgactactacaaggtgtccaggctgag
SEQ ID NO: 4
Exemplary Leader Sequence for Llama Single Chain Antibody (amino acid)
M E L G L S W V V L A A L L Q G V Q A E
SEQ ID NO: 5
Exemplary variable domain and CH1 domain of first heavy chain of Rabbit Antibody
(nucleotide)
cagtcgctggaggagtccgggggtcgcctggtcacgcctgggacacccctgacactcacctgcacagtactggaatcgacctc
acttcctactacatgacctgggtccgccagggtccagggaaggggctggaatggatcggcatcattagtactgatggtagcgc
atcctacgcgaactgggcgaaaggccgattcaccataccaaaacctcgaccacggtggatctgaaaatcaccagtccgacaac
cgacgacacggccacctatttttgtgccagagcttacgatacttatggttcgggttatgatggtgtattggcatctggggccc
aggcaccaggtcaccgtacctcagggcaacctaaggctccatcagtcttcccactggccccctgctgcggggacacacccagc
tccacggtgaccctgggctgcctggtcaaaggctacctcccggagccagtgaccgtgacctggaactcgggcaccctcaccaa
tggggtacgcaccttcccgtccgtccggcagtcctcaggcctctactcgctgagcagcgtggtgagcgtgacctcaagcagcc
agcccgtcacctgcaacgtggcccacccagccaccaacaccaaagtggacaagaccgttgcgccctcgacatgcagcaagccc
acgtgcccaccccctgaactcctg
SEQ ID NO: 6
Exemplary variable domain and CH1 domain of first heavy chain of Rabbit Antibody
(amino acid)
Q S L E E S G G R L V T P G T P L T L T C T V S G I D L T S Y Y M T W V R Q G P G K
G L E W I G I I S T D G S A S Y A N W A K G R F T I S K T S T T V D L K I T S P T T
D D T A T Y F C A R A Y D T Y G S G Y D G V F G I W G P G T L V T V S S G Q P K A P
S V F P L A P C C G D T P S S T V T L G C L V K G Y L P E P V T V T W N S G T L T N
G V R T F P S V R Q S S G L Y S L S S V V S V T S S S Q P V T C N V A H P A T N T K
V D K T V A P S T C S K P T C P P P E L L
SEQ ID NO: 7
Exemplary variable domain and CH1 domain of first heavy chain of Llama Antibody
(nucleotide)
gtgcagctggtggagtctgggggaggcttggtgcggcctggggggtctctgagactcatctgtgaagcctttggaagcatctt
cagcgtgaataccatgggctggtaccgccagaccccggggaagcagcgggagttggtcgcaattatgactagtgaaggtacga
caaattatgcagatttcgtgaagggccgattcaccatctccagagacaacgccaaggacacagtgtatctacaaatgaacagc
ctgaattctgaggacacggccgtctattattgtctcttgaaattgccatttacccttaaggagtattggggccaggggaccca
ggtcaccgtctccgcagaacccaagacaccaaaa
SEQ ID NO: 8
Exemplary variable domain and CH1 domain of first heavy chain of Llama Antibody
(amino acid)
V Q L V E S G G G L V R P G G S L R L I C E A F G S I F S V N T M G W Y R Q T P G K
Q R E L V A I M T S E G T T N Y A D F V K G R F T I S R D N A K D T V Y L Q M N S L
N S E D T A V Y Y C L L K L P F T L K E Y W G Q G T Q V T V S A E P K T P K
SEQ ID NO: 9
Exemplary Linker (nucleotide)
cctcaaccccaaccacaaccgcaacctcagccccagccacagccgcagccgcagccacaaccccagcctcaaccacaacccca
gcctcaacctcag
SEQ ID NO: 10
Exemplary Linker (amino acid)
P Q P Q P Q P Q P Q P Q P Q P Q P Q P Q P Q P Q P Q P Q P Q P Q
SEQ ID NO: 11
Exemplary variable domain and CH1 domain, hinge region, and CH2 and CH3 domains
of Second Heavy Chain-Rabbit Antibody (nucleotide)
cagtcgctggaggagtccgggggtcgcctggtcacgcctgggacacccctgacactcacctgcacagtctctggaatcgacct
cacttcctactacatgacctgggtccgccagggtccagggaaggggctggaatggatcggcatcattagtactgatggtagcg
catcctacgcgaactgggcgaaaggccgattcaccatctccaaaacctcgaccacggtggatctgaaaatcaccagtccgaca
accgacgacacggccacctatttttgtgccagagcttacgatacttatggttcgggttatgatggtgtctttggcatctgggg
cccaggcaccctggtcaccgtctcctcagggcaacctaaggctccatcagtcttcccactggccccctgctgcggggacacac
ccagctccacggtgaccctgggctgcctggtcaaaggctacctcccggagccagtgaccgtgacctggaactcgggcaccctc
accaatggggtacgcaccttcccgtccgtccggcagtcctcaggcctctactcgctgagcagcgtggtgagcgtgacctcaag
cagccagcccgtcacctgcaacgtggcccacccagccaccaacaccaaagtggacaagaccgttgcgccctcgacatgcagca
agcccacgtgcccaccccctgaactcctggggggaccgtctgtcttcatcttccccccaaaacccaaggacaccctcatgatc
tcacgcacccccgaggtcacatgcgtggtggtggacgtgagccaggatgaccccgaggtgcagttcacatggtacataaacaa
cgagcaggtgcgcaccgcccggccgccgctacgggagcagcagttcaacagcacgatccgcgtggtcagcaccctccccatcg
cgcaccaggactggctgaggggcaaggagttcaagtgcaaagtccacaacaaggcactcccggcccccatcgagaaaaccatc
tccaaagccagagggcagcccctggagccgaaggtctacaccatgggccctccccgggaggagctgagcagcaggtcggtcag
cctggcctgcatgatcaacggcttctacccttccgacatctcggtggagtgggagaagaacgggaaggcagaggacaactaca
agaccacgccggccgtgctggacagcgacggctcctacttcctctacagcaagctctcagtgcccacgagtgagtggcagcgg
ggcgacgtcttcacctgctccgtgatgcacgaggccttgcacaaccactacacgcagaagtccatctcccgctctccgggtaa
atga
SEQ ID NO: 12
Exemplary variable domain and CH1 domain, hinge region, and CH2 and CH3 domains
of Second Heavy Chain-Rabbit Antibody (amino acid)
Q S L E E S G G R L V T P G T P L T L T C T V S G I D L T S Y Y M T W V R Q G P G K
G L E W I G I I S T D G S A S Y A N W A K G R F T I S K T S T T V D L K I T S P T T
D D T A T Y F C A R A Y D T Y G S G Y D G V F G I W G P G T L V T V S S G Q P K A P
S V F P L A P C C G D T P S S T V T L G C L V K G Y L P E P V T V T W N S G T L T N
G V R T F P S V R Q S S G L Y S L S S V V S V T S S S Q P V T C N V A H P A T N T K
V D K T V A P S T C S K P T C P P P E L L G G P S V F I F P P K P K D T L M I S R T
P E V T C V V V D V S Q D D P E V Q F T W Y I N N E Q V R T A R P P L R E Q Q F N S
T I R V V S T L P I A H Q D W L R G K E F K C K V H N K A L P A P I E K T I S K A R
G Q P L E P K V Y T M G P P R E E L S S R S V S L A C M I N G F Y P S D I S V E W E
K N G K A E D N Y K T T P A V L D S D G S Y F L Y S K L S V P T S E W Q R G D V F T
C S V M H E A L H N H Y T Q K S I S R S P G K -
SEQ ID NO: 13
Exemplary variable domain and CH1 domain, hinge region, and CH2 and CH3 domains
of Second Heavy Chain-Llama Antibody (nucleotide)
gctcaggtgcagctggtggagtctgggggaggcttggtgcagcctggggggtctctgaggctctcctgtgtagcctctggaaa
catgttcggcattaacacgatgggctggttccgcgcggctccagggaaagagcgtcagtttgtagcagctgataatttcagtg
gtggcaggaagaatattgacttctccgtgaggggtcgattcaccatgtcaagagacgccgccagcaacacgatatatctacaa
atgaacagcctgaaagttgaggacacggccgtttattactgcgcagcgggtgcgaatacgatgatgattgtggcggacaatta
cgaatactggggtcaggggacccaggtcaccgtctcctcagaacccaagacaccaaaaaagcttccacagcctcagccgcagc
cccagccacaaccacaacccaatcctacaacagaatccaagtgtcccaaatgtccagcccctgagctcctgggagggccdcag
tcttcatcttccccccgaaacccaaggacgtcctctccatttctgggaggcccgaggtcacgtgcgttgtggtagacgtgggc
caggaagaccccgaggtcagtttcaactggtacattgatggcgctgaggtgcgaacggccaacacgaggccaaaagaggaaca
gttcaacagcacgtaccgcgtggtcagcgtcctgcccatccagcaccaggactggctgacggggaaggaattcaagtgcaagg
tcaacaacaaagctctcccggcccccatcgagaagaccatctccaaggccaaagggcagacccgggagccgcaggtgtacgcc
ctggccccacaccgggaagagctggccaaggacaccgtgagcgtaacctgcctggtcaaaggcttctacccacctgatatcaa
cgttgagtggcagaggaacggtcagccggagtcagagggcacctacgccaccacgccaccccagctggacaacgacgggacct
acttcctctacagcaagctctcggtgggaaagaacacgtggcagcggggagaaaccttcacctgtgtggtgatgcatgaggcc
ctgtga
SEQ ID NO: 14
Exemplary variable domain and CH1 domain, hinge region, and CH2 and CH3 domains
of Second Heavy Chain-Llama Antibody (amino acid)
A Q V Q L V E S G G G L V Q P G G S L R L S C V A S G N M F G I N T M G W F R A A P
G K E R Q F V A A D N F S G G R K N I D F S V R G R F T M S R D A A S N T I Y L Q M
N S L K V E D T A V Y Y C A A G A N T M M I V A D N Y E Y W G Q G T Q V T V S S E P
K T P K K L P Q P Q P Q P Q P Q P Q P N P T T E S K C P K C P A P E L L G G P S V F
I F P P K P K D V L S I S G R P E V T C V V V D V G Q E D P E V S F N W Y I D G A E
V R T A N T R P K E E Q F N S T Y R V V S V L P I Q H Q D W L T G K E F K C K V N N
K A L P A P I E K T I S K A K G Q T R E P Q V Y A L A P H R E E L A K D T V S V T C
L V K G F Y P P D I N V E W Q R N G Q P E S E G T Y A T T P P Q L D N D G T Y F L Y
S K L S V G K N T W Q R G E T F T C V V M H E A L -
SEQ ID NO: 15
Exemplary Construct Containing Variations of C-terminus (and corresponding 3′ DNA
sequences) of Second Single Heavy Chain-his-tag fused to End of CH3 domain
(nucleotide):
gctcaggtgcagctggtggagtctgggggaggcttggtgcagcctggggggtctctgaggctctcctgtgtagcctctggaaa
catgttcggcattaacacgatgggctggttccgcgcggctccagggaaagagcgtcagtttgtagcagctgataatttcagtg
gtggcaggaagaatattgacttctccgtgaggggtcgattcaccatgtcaagagacgccgccagcaacacgatatatctacaa
atgaacagcctgaaagttgaggacacggccgtttattactgcgcagcgggtgcgaatacgatgatgattgtggcggacaatta
cgaatactggggtcaggggacccaggtcaccgtctcctcagaacccaagacaccaaaaaagcttccacagcctcagccgcagc
cccagccacaaccacaacccaatcctacaacagaatccaagtgtcccaaatgtccagcccctgagctcctgggagggccdcag
tcttcatcttccccccgaaacccaaggacgtcctctccatttctgggaggcccgaggtcacgtgcgttgtggtagacgtgggc
caggaagaccccgaggtcagtttcaactggtacattgatggcgctgaggtgcgaacggccaacacgaggccaaaagaggaaca
gttcaacagcacgtaccgcgtggtcagcgtcctgcccatccagcaccaggactggctgacggggaaggaattcaagtgcaagg
tcaacaacaaagctctcccggcccccatcgagaagaccatctccaaggccaaagggcagacccgggagccgcaggtgtacgcc
ctggccccacaccgggaagagctggccaaggacaccgtgagcgtaacctgcctggtcaaaggcttctacccacctgatatcaa
cgttgagtggcagaggaacggtcagccggagtcagagggcacctacgccaccacgccaccccagctggacaacgacgggacct
acttcctctacagcaagctctcggtgggaaagaacacgtggcagcggggagaaaccttcacctgtgtggtgatgcatgaggcc
ctggttccctctagagggcccttcgaaggtaagcctatccctaaccctctcctcggtctcgattctacgcgtaccggtcatca
tcaccatcaccatcaccatggaggacagtga
SEQ ID NO: 16
Exemplary Construct Containing Variations of C-terminus (and corresponding 3′ DNA
sequences) of Second Single Heavy Chain-his-tag fused to End of CH3 domain (amino
acid):
A Q V Q L V E S G G G L V Q P G G S L R L S C V A S G N M F G I N T M G W F R A A P
G K E R Q F V A A D N F S G G R K N I D F S V R G R F T M S R D A A S N T I Y L Q M
N S L K V E D T A V Y Y C A A G A N T M M I V A D N Y E Y W G Q G T Q V T V S S E P
K T P K K L P Q P Q P Q P Q P Q P Q P N P T T E S K C P K C P A P E L L G G P S V F
I F P P K P K D V L S I S G R P E V T C V V V D V G Q E D P E V S F N W Y I D G A E
V R T A N T R P K E E Q F N S T Y R V V S V L P I Q H Q D W L T G K E F K C K V N N
K A L P A P I E K T I S K A K G Q T R E P Q V Y A L A P H R E E L A K D T V S V T C
L V K G F Y P P D I N V E W Q R N G Q P E S E G T Y A T T P P Q L D N D G T Y F L Y
S K L S V G K N T W Q R G E T F T C V V M H E A L V P S R G P F E G K P I P N P L L
G L D S T R T G H H H H H H H H G G Q -
SEQ ID NO: 17
Exemplary Construct Containing Variations of C-terminus (and corresponding 3′ DNA
sequences) of Second Single Heavy Chain-his-tag fused to CH1 domain (nucleotide)
gtgcagctggtggagtctgggggaggcttggtgcagcctggggggtctctgaggctctcctgtgtagcctctggaaacatgtt
cggcattaacacgatgggctggttccgcgcggctccagggaaagagcgtcagtttgtagcagctgataatttcagtggtggca
ggaagaatattgacttctccgtgaggggtcgattcaccatgtcaagagacgccgccagcaacacgatatatctacaaatgaac
agcctgaaagttgaggacacggccgtttattactgcgcagcgggtgcgaatacgatgatgattgtggcggacaattacgaata
ctggggtcaggggacccaggtcaccgtctcctcagaacccaagacaccaaaaaagcttgatatcacgcgtaccggtcatcatc
accatcaccatcaccatggaggacagtgaagatcccccgacctcgacctctggctaataaaggaa
SEQ ID NO: 18
Exemplary Construct Containing Variations of C-terminus (and corresponding 3′ DNA
sequences) of Second Single Heavy Chain-his-tag fused to CH1 domain (amino acid)
V Q L V E S G G G L V Q P G G S L R L S C V A S G N M F G I N T M G W F R A A P G K
E R Q F V A A D N F S G G R K N I D F S V R G R F T M S R D A A S N T I Y L Q M N S
L K V E D T A V Y Y C A A G A N T M M I V A D N Y E Y W G Q G T Q V T V S S E P K T
P K K L D I T R T G H H H H H H H H G G Q -
SEQ ID NO: 19
Heavy Chain Construct 1 (nucleotide) containing heavy chain sequence (Antibody 1)
linked and in-frame with second heavy chain sequence (Antibody 1). Bold,
underlined, and italicized DNA sequences are restriction sites used for subcloning
to build the constructs (nucleotide) [symbol]
gacgctcaccatggagactgggctgcgctggcttctcctggtcgctgtgctcaaaggtgtccagtgtcagtcggtgg
aggagtccgggggtcgcctggtcacgcctgggacacccctgacactcacctgcacagtctctggattctccctcaataccaat
gcaataaggtgggtccgccaggctccagggaaggggctggaatggatcggagccattgatgctagtggtaacacatactacgc
gagctgggcgaaaggccgattcaccatctccaaaacctcgaccacggtggatctgaaaatcaccagtccgacaaccgaggaca
cggccacctatttctgtaacagatttaatactgccaacatctggggcccaggcaccctggtcaccgtctcctcagggcaacct
aaggctccatcagtcttcccactggccccctgctgcggggacacacccagctccacggtgaccctgggctgcctggtcaaagg
ctacctcccggagccagtgaccgtgacctggaactcgggcaccctcaccaatggggtacgcaccttcccgtccgtccggcagt
cctcaggcctctactcgctgagcagcgtggtgagcgtgacctcaagcagccagcccgtcacctgcaacgtggcccacccagcc
accaacaccaaagtggacaagaccgttgcgccctcgacatgcagcaagcccatgtgcccaccccctgaactcctgg [symbol]
cctcaaccccaaccacaaccgcaacctcagccccagccacagccgcagccgcagccacaaccccagcctcaaccacaacccca
gcctcaacctcag [symbol] cagtcggtggaggagtccgggggtcgcctggtcacgcctgggacacccctgacactcacctgca
cagtctctggattctccctcaataccaatgcaataaggtgggtccgccaggctccagggaaggggctggaatggatcggagcc
attgatgctagtggtaacacatactacgcgagctgggcgaaaggccgattcaccatctccaaaacctcgaccacggtggatct
gaaaatcaccagtccgacaaccgaggacacggccacctatttctgtaacagatttaatactgccaacatctggggcccaggca
ccctggtcaccgtctcctcagggcaacctaaggctccatcagtcttcccactggccccctgctgcggggacacacccagctcc
acggtgaccctgggctgcctggtcaaaggctacctcccggagccagtgaccgtgacctggaactcgggcaccctcaccaatgg
ggtacgcaccttcccgtccgtccggcagtcctcaggcctctactcgctgagcagcgtggtgagcgtgacctcaagcagccagc
ccgtcacctgcaacgtggcccacccagccaccaacaccaaagtggacaagaccgttgcgccctcgacatgcagcaagcccacg
tgcccaccccctgaactcctggggggaccgtctgtcttcatcttccccccaaaacccaaggacaccctcatgatctcacgcac
ccccgaggtcacatgcgtggtggtggacgtgagccaggatgaccccgaggtgcagttcacatggtacataaacaacgagcagg
tgcgcaccgcccggccgccgctacgggagcagcagttcaacagcacgatccgcgtggtcagcaccctccccatcgcgcaccag
gactggctgaggggcaaggagttcaagtgcaaagtccacaacaaggcactcccggcccccatcgagaaaaccatctccaaagc
cagagggcagcccctggagccgaaggtctacaccatgggccctccccgggaggagctgagcagcaggtcggtcagcctggcct
gcatgatcaacggatctacccttccgacatctcggtggagtgggagaagaacgggaaggcagaggacaactacaagaccacgc
cggccgtgctggacagcgacggctcctacttcctctacagcaagctctcagtgcccacgagtgagtggcagcggggcgacgta
tcacctgctccgtgatgcacgaggccttgcacaaccactacacgcagaagtccatctcccgctctccgggtaaatga [symbol]
SEQ ID NO: 20
Heavy Chain Construct 1 (amino acid) containing heavy chain sequence (Antibody 1)
linked and in-frame with second heavy chain sequence (Antibody 1). Italicized amino
acid sequence is signal peptide. Underlined amino acid sequences are two variable
domains in tandem of Antibody 1. Bold amino acid sequence is PQ Linker.
M E T G L R W L L L V A V L K G V Q C Q S V E E S G G R L V T P G T P L T L T C T V
S G F S L N T N A I R W V R Q A P G K G L E W I G A I D A S G N T Y Y A S W A K G R
F T I S K T S T T V D L K I T S P T T E D T A T Y F C N R F N T A N I W G P G T L V
T V S S G Q P K A P S V F P L A P C C G D T P S S T V T L G C L V K G Y L P E P V T
V T W N S G T L T N G V R T F P S V R Q S S G L Y S L S S V V S V T S S S Q P V T C
N V A H P A T N T K V D K T V A P S T C S K P M C P P P E L L G G R P Q P Q P Q P Q
P Q P Q P Q P Q P Q P Q P Q P Q P Q P Q P Q P Q S R Q S V E E S G G R L V T P G T P
L T L T C T V S G F S L N T N A I R W V R Q A P G K G L E W I G A I D A S G N T Y Y
A S W A K G R F T I S K T S T T V D L K I T S P T T E D T A T Y F C N R F N T A N I
W G P G T L V T V S S G Q P K A P S V F P L A P C C G D T P S S T V T L G C L V K G
Y L P E P V T V T W N S G T L T N G V R T F P S V R Q S S G L Y S L S S V V S V T S
S S Q P V T C N V A H P A T N T K V D K T V A P S T C S K P T C P P P E L L G G P S
V F I F P P K P K D T L M I S R T P E V T C V V V D V S Q D D P E V Q F T W Y I N N
E Q V R T A R P P L R E Q Q F N S T I R V V S T L P I A H Q D W L R G K E F K C K V
H N K A L P A P I E K T I S K A R G Q P L E P K V Y T M G P P R E E L S S R S V S L
A C M I N G F Y P S D I S V E W E K N G K A E D N Y K T T P A V L D S D G S Y F L Y
S K L S V P T S E W Q R G D V F T C S V M H E A L H N H Y T Q K S I S R S P G K -
SEQ ID NO: 21
Heavy Chain Construct 1 (amino acid) containing heavy chain sequence (Antibody 1)
linked and in-frame with second heavy chain sequence (Antibody 1)-without signal
peptide. Underlined amino acid sequences are two variable domains in tandem of
Antibody 1. Bold amino acid sequence is PQ Linker.
Q S V E E S G G R L V T P G T P L T L T C T V S G F S L N T N A I R W V R Q A P G K
G L E W I G A I D A S G N T Y Y A S W A K G R F T I S K T S T T V D L K I T S P T T
E D T A T Y F C N R F N T A N I W G P G T L V T V S S G Q P K A P S V F P L A P C C
G D T P S S T V T L G C L V K G Y L P E P V T V T W N S G T L T N G V R T F P S V R
Q S S G L Y S L S S V V S V T S S S Q P V T C N V A H P A T N T K V D K T V A P S T
C S K P M C P P P E L L G G R P Q P Q P Q P Q P Q P Q P Q P Q P Q P Q P Q P Q P Q P
Q P Q P Q S R Q S V E E S G L N T N A I R W V R Q A P G K G L E W I G A I D A S G N
T Y Y A S W A K G R F T I S K T S T T V D L K I T S P T T E D T A T Y F C N R F N T
A N I W G P G T L V T V S S G Q P K A P S V F P L A P C C G D T P S S T V T L G C L
V K G Y L P E P V T V T W N S G T L T N G V R T F P S V R Q S S G L Y S L S S V V S
V T S S S Q P V T C N V A H P A T N T K V D K T V A P S T C S K P T C P P P E L L G
G P S V F I F P P K P K D T L M I S R T P E V T C V V V D V S Q D D P E V Q F T W Y
I N N E Q V R T A R P P L R E Q Q F N S T I R V V S T L P I A H Q D W L R G K E F K
C K V H N K A L P A P I E K T I S K A R G Q P L E P K V Y T M G P P R E E L S S R S
V S L A C M I N G F Y P S D I S V E W E K N G K A E D N Y K T T P A V L D S D G S Y
F L Y S K L S V P T S E W Q R G D V F T C S V M H E A L H N H Y T Q K S I S R S P G
K -
SEQ ID NO: 22
Heavy Chain Construct 2 (nucleotide) containing heavy chain sequence (Antibody 2)
linked and in-frame with second heavy chain sequence (Antibody 2). Bold,
underlined, and italicized DNA sequences are restriction sites used for subcloning
to build the constructs (nucleotide) [symbol]
gacgctcaccatggagactgggctgcgctggcttctcctggtcgctgtgctcaaaggtgtccagtgtcagtcgctgg
aggagtccgggggtcgcctggtctcgcctgggacacccctgacactcacctgcaccgcctctggattctccctcagtagcaat
gcaatgggctgggtccgccaggctccaggggaggggctggaatacatcggaatcattaggcctggtggtagcacatactacgc
gagctgggcgaaaggccgattcaccatctccaaaacctcgtcgaccacggtggatctgaaaatgaccagtctgacaaccgagg
acacggccacctatttctgtgccagagcctgggacatctggggcccaggcaccctggtcaccgtctccttagggcaacctaag
gctccatcagtcttcccactggccccctgctgcggggacacacccagctccacggtgaccctgggctgcctggtcaaaggcta
cctcccggagccagtgaccgtgacctggaactcgggcaccctcaccaatggggtacgcaccttcccgtccgtccggcagtcct
caggcctctactcgctgagcagcgtggtgagcgtgacctcaagcagccagcccgtcacctgcaacgtggcccacccagccacc
aacaccaaagtggacaagaccgttgcgccctcgacatgcagcaagcccacgtgcccaccccctgaactcctgg [symbol] ct
caaccccaaccacaaccgcaacctcagccccagccacagccgcagccgcagccacaaccccagcctcaaccacaaccccagcc
tcaacctcag [symbol] cagtcgctggaggagtccgggggtcgcctggtctcgcctgggacacccctgacactcacctgcaccg
cctctggattctccctcagtagcaatgcaatgggctgggtccgccaggctccaggggaggggctggaatacatcggaatcatt
aggcctggtggtagcacatactacgcgagctgggcgaaaggccgattcaccatctccaaaacctcgtcgaccacggtggatct
gaaaatgaccagtctgacaaccgaggacacggccacctatttctgtgccagagcctgggacatctggggcccaggcaccctgg
tcaccgtctccttagggcaacctaaggctccatcagtcttcccactggccccctgctgcggggacacacccagaccacggtga
ccagggctgcctggtcaaaggctacctcccggagccagtgaccgtgacctggaactcgggcaccacaccaatggggtacgcac
cttcccgtccgtccggcagtcctcaggcctctactcgctgagcagcgtggtgagcgtgacctcaagcagccagcccgtcacct
gcaacgtggcccacccagccaccaacaccaaagtggacaagaccgttgcgccacgacatgcagcaagcccacgtgcccacccc
ctgaactcctggggggaccgtagtatcatcttccccccaaaacccaaggacaccacatgatacacgcacccccgaggtcacat
gcgtggtggtggacgtgagccaggatgaccccgaggtgcagttcacatggtacataaacaacgagcaggtgcgcaccgcccgg
ccgccgctacgggagcagcagttcaacagcacgatccgcgtggtcagcaccaccccatcgcgcaccaggactggctgaggggc
aaggagttcaagtgcaaagtccacaacaaggcactcccggcccccatcgagaaaaccatctccaaagccagagggcagcccct
ggagccgaaggtctacaccatgggccaccccgggaggagagagcagcaggtcggtcagcctggcctgcatgatcaacggatct
accatccgacatctcggtggagtgggagaagaacgggaaggcagaggacaactacaagaccacgccggccgtgctggacagcg
acggctcctacttcctctacagcaagactcagtgcccacgagtgagtggcagcggggcgacgtcttcacctgaccgtgatgca
cgaggccttgcacaaccactacacgcagaagtccatacccgctaccgggtaaatga [symbol]
SEQ ID NO: 23
Italicized amino acid sequence is signal peptide. Underlined amino acid sequences
are two variable domains in tandem of Antibody 2. Bold amino acid sequence is PQ
Linker.
M E T G L R W L L L V A V L K G V Q C Q S L E E S G G R L V S P G T P L T L T C T A
S G F S L S S N A M G W V R Q A P G E G L E Y I G I I R P G G S T Y Y A S W A K G R
F T I S K T S S T T V D L K M T S L T T E D T A T Y F C A R A W D I W G P G T L V T
V S L G Q P K A P S V F P L A P C C G D T P S S T V T L G C L V K G Y L P E P V T V
T W N S G T L T N G V R T F P S V R Q S S G L Y S L S S V V S V T S S S Q P V T C N
V A H P A T N T K V D K T V A P S T C S K P T C P P P E L L G G R P Q P Q P Q P Q P
Q P Q P Q P Q P Q P Q P Q P Q P Q P Q P Q P Q S R Q S L E E S G G R L V S P G T P L
T L T C T A S G F S L S S N A M G W V R Q A P G E G L E Y I G I I R P G G S T Y Y A
S W A K G R F T I S K T S S T T V D L K M T S L T T E D T A T Y F C A R A W D I W G
P G T L V T V S L G Q P K A P S V F P L A P C C G D T P S S T V T L G C L V K G Y L
P E P V T V T W N S G T L T N G V R T F P S V R Q S S G L Y S L S S V V S V T S S S
Q P V T C N V A H P A T N T K V D K T V A P S T C S K P T C P P P E L L G G P S V F
I F P P K P K D T L M I S R T P E V T C V V V D V S Q D D P E V Q F T W Y I N N E Q
V R T A R P P L R E Q Q F N S T I R V V S T L P I A H Q D W L R G K E F K C K V H N
K A L P A P I E K T I S K A R G Q P L E P K V Y T M G P P R E E L S S R S V S L A C
M I N G F Y P S D I S V E W E K N G K A E D N Y K T T P A V L D S D G S Y F L Y S K
L S V P T S E W Q R G D V F T C S V M H E A L H N H Y T Q K S I S R S P G K -
SEQ ID NO: 24
Heavy Chain Construct 2 (amino acid) containing heavy chain sequence (Antibody 2)
linked and in-frame with second heavy chain sequence (Antibody 2)-without signal
peptide. Underlined amino acid sequences are two variable domains in tandem of
Antibody 2. Bold amino acid sequence is PQ Linker.
Q S L E E S G G R L V S P G T P L T L T C T A S G F S L S S N A M G W V R Q A P G E
G L E Y I G I I R P G G S T Y Y A S W A K G R F T I S K T S S T T V D L K M T S L T
T E D T A T Y F C A R A W D I W G P G T L V T V S L G Q P K A P S V F P L A P C C G
D T P S S T V T L G C L V K G Y L P E P V T V T W N S G T L T N G V R T F P S V R Q
S S G L Y S L S S V V S V T S S S Q P V T C N V A H P A T N T K V D K T V A P S T C
S K P T C P P P E L L G G R P Q P Q P Q P Q P Q P Q P Q P Q P Q P Q P Q P Q P Q P Q
P Q P Q S R Q S L E E S G G R L V S P G T P L T L T C T A S G F S L S S N A M G W V
R Q A P G E G L E Y I G I I R P G G S T Y Y A S W A K G R F T I S K T S S T T V D L
K M T S L T T E D T A T Y F C A R A W D I W G P G T L V T V S L G Q P K A P S V F P
L A P C C G D T P S S T V T L G C L V K G Y L P E P V T V T W N S G T L T N G V R T
F P S V R Q S S G L Y S L S S V V S V T S S S Q P V T C N V A H P A T N T K V D K T
V A P S T C S K P T C P P P E L L G G P S V F I F P P K P K D T L M I S R T P E V T
C V V V D V S Q D D P E V Q F T W Y I N N E Q V R T A R P P L R E Q Q F N S T I R V
V S T L P I A H Q D W L R G K E F K C K V H N K A L P A P I E K T I S K A R G Q P L
E P K V Y T M G P P R E E L S S R S V S L A C M I N G F Y P S D I S V E W E K N G K
A E D N Y K T T P A V L D S D G S Y F L Y S K L S V P T S E W Q R G D V F T C S V M
H E A L H N H Y T Q K S I S R S P G K -
SEQ ID NO: 25
Heavy Chain Construct 3 (nucleotide) containing heavy chain sequence (Antibody 1)
linked and in-frame with second heavy chain sequence (Antibody 2). Bold,
underlined, and italicized DNA sequences are restriction sites used for subcloning
to build the constructs (nucleotide) [symbol]
gacgctcaccatggagactgggctgcgctggcttctcctggtcgctgtgctcaaaggtgtccagtgtcagtcggtgg
aggagtccgggggtcgcctggtcacgcctgggacacccctgacactcacctgcacagtctctggattctccctcaataccaat
gcaataaggtgggtccgccaggctccagggaaggggctggaatggatcggagccattgatgctagtggtaacacatactacgc
gagctgggcgaaaggccgattcaccatctccaaaacctcgaccacggtggatctgaaaatcaccagtccgacaaccgaggaca
cggccacctatttctgtaacagatttaatactgccaacatctggggcccaggcaccctggtcaccgtctcctcagggcaacct
aaggctccatcagtcttcccactggccccctgctgcggggacacacccagctccacggtgaccctgggctgcctggtcaaagg
ctacctcccggagccagtgaccgtgacctggaactcgggcaccctcaccaatggggtacgcaccttcccgtccgtccggcagt
cctcaggcctctactcgctgagcagcgtggtgagcgtgacctcaagcagccagcccgtcacctgcaacgtggcccacccagcc
accaacaccaaagtggacaagaccgttgcgccctcgacatgcagcaagcccatgtgcccaccccctgaactcctgg [symbol]
cctcaaccccaaccacaaccgcaacctcagccccagccacagccgcagccgcagccacaaccccagcctcaaccacaacccca
gcctcaacctcag [symbol] cagtcgctggaggagtccgggggtcgcctggtctcgcctgggacacccctgacactcacctgca
ccgcctctggattctccctcagtagcaatgcaatgggctgggtccgccaggctccaggggaggggctggaatacatcggaatc
attaggcctggtggtagcacatactacgcgagctgggcgaaaggccgattcaccatctccaaaacctcgtcgaccacggtgga
tctgaaaatgaccagtctgacaaccgaggacacggccacctatttctgtgccagagcctgggacatctggggcccaggcaccc
tggtcaccgtctccttagggcaacctaaggctccatcagtcttcccactggccccctgctgcggggacacacccagctccacg
gtgaccctgggctgcctggtcaaaggctacctcccggagccagtgaccgtgacctggaactcgggcaccctcaccaatggggt
acgcaccttcccgtccgtccggcagtcctcaggcctctactcgctgagcagcgtggtgagcgtgacctcaagcagccagcccg
tcacctgcaacgtggcccacccagccaccaacaccaaagtggacaagaccgttgcgccctcgacatgcagcaagcccacgtgc
ccaccccctgaactcctggggggaccgtctgtcttcatcttccccccaaaacccaaggacaccctcatgatctcacgcacccc
cgaggtcacatgcgtggtggtggacgtgagccaggatgaccccgaggtgcagttcacatggtacataaacaacgagcaggtgc
gcaccgcccggccgccgctacgggagcagcagttcaacagcacgatccgcgtggtcagcaccctccccatcgcgcaccaggac
tggctgaggggcaaggagttcaagtgcaaagtccacaacaaggcactcccggcccccatcgagaaaaccatctccaaagccag
agggcagcccctggagccgaaggtctacaccatgggccctccccgggaggagctgagcagcaggtcggtcagcctggcctgca
tgatcaacggatctacccttccgacatctcggtggagtgggagaagaacgggaaggcagaggacaactacaagaccacgccgg
ccgtgctggacagcgacggctcctacttcctctacagcaagctctcagtgcccacgagtgagtggcagcggggcgacgtcttc
acctgaccgtgatgcacgaggccttgcacaaccactacacgcagaagtccatctcccgctaccgggtaaatga [symbol]
SEQ ID NO: 26
Heavy Chain Construct 3 (amino acid) containing heavy chain sequence (Antibody 1)
linked and in-frame with second heavy chain sequence (Antibody 2). Italicized amino
acid sequence is signal peptide. Underlined amino acid sequences are two variable
domains in tandem of Antibody 1 and Antibody 2, respectively. Bold amino acid
sequence is PQ Linker.
M E T G L R W L L L V A V L K G V Q C Q S V E E S G G R L V T P G T P L T L T C T V
S G F S L N T N A I R W V R Q A P G K G L E W I G A I D A S G N T Y Y A S W A K G R
F T I S K T S T T V D L K I T S P T T E D T A T Y F C N R F N T A N I W G P G T L V
T V S S G Q P K A P S V F P L A P C C G D T P S S T V T L G C L V K G Y L P E P V T
V T W N S G T L T N G V R T F P S V R Q S S G L Y S L S S V V S V T S S S Q P V T C
N V A H P A T N T K V D K T V A P S T C S K P M C P P P E L L G G R P Q P Q P Q P Q
P Q P Q P Q P Q P Q P Q P Q P Q P Q P Q P Q P Q S R Q S L E E S G G R L V S P G T P
L T L T C T A S G F S L S S N A M G W V R Q A P G E G L E Y I G I I R P G G S T Y Y
A S W A K G R F T I S K T S S T T V D L K M T S L T T E D T A T Y F C A R A W D I W
G P G T L V T V S L G Q P K A P S V F P L A P C C G D T P S S T V T L G C L V K G Y
L P E P V T V T W N S G T L T N G V R T F P S V R Q S S G L Y S L S S V V S V T S S
S Q P V T C N V A H P A T N T K V D K T V A P S T C S K P T C P P P E L L G G P S V
F I F P P K P K D T L M I S R T P E V T C V V V D V S Q D D P E V Q F T W Y I N N E
Q V R T A R P P L R E Q Q F N S T I R V V S T L P I A H Q D W L R G K E F K C K V H
N K A L P A P I E K T I S K A R G Q P L E P K V Y T M G P P R E E L S S R S V S L A
C M I N G F Y P S D I S V E W E K N G K A E D N Y K T T P A V L D S D G S Y F L Y S
K L S V P T S E W Q R G D V F T C S V M H E A L H N H Y T Q K S I S R S P G K -
SEQ ID NO: 27
Heavy Chain Construct 3 (amino acid) containing heavy chain sequence (Antibody 1)
linked and in-frame with second heavy chain sequence (Antibody 2)-without signal
peptide. Underlined amino acid sequences are two variable domains in tandem of
Antibody 1 and Antibody 2, respectively. Bold amino acid sequence is PQ Linker.
Q S V E E S G G R L V T P G T P L T L T C T V S G F S L N T N A I R W V R Q A P G K
G L E W I G A I D A S G N T Y Y A S W A K G R F T I S K T S T T V D L K I T S P T T
E D T A T Y F C N R F N T A N I W G P G T L V T V S S G Q P K A P S V F P L A P C C
G D T P S S T V T L G C L V K G Y L P E P V T V T W N S G T L T N G V R T F P S V R
Q S S G L Y S L S S V V S V T S S S Q P V T C N V A H P A T N T K V D K T V A P S T
C S K P M C P P P E L L G G R P Q P Q P Q P Q P Q P Q P Q P Q P Q P Q P Q P Q P Q P
Q P Q P Q S R Q S L E E S G G R L V S P G T P L T L T C T A S G F S L S S N A M G W
V R Q A P G E G L E Y I G I I R P G G S T Y Y A S W A K G R F T I S K T S S T T V D
L K M T S L T T E D T A T Y F C A R A W D I W G P G T L V T V S L G Q P K A P S V F
P L A P C C G D T P S S T V T L G C L V K G Y L P E P V T V T W N S G T L T N G V R
T F P S V R Q S S G L Y S L S S V V S V T S S S Q P V T C N V A H P A T N T K V D K
T V A P S T C S K P T C P P P E L L G G P S V F I F P P K P K D T L M I S R T P E V
T C V V V D V S Q D D P E V Q F T W Y I N N E Q V R T A R P P L R E Q Q F N S T I R
V V S T L P I A H Q D W L R G K E F K C K V H N K A L P A P I E K T I S K A R G Q P
L E P K V Y T M G P P R E E L S S R S V S L A C M I N G F Y P S D I S V E W E K N G
K A E D N Y K T T P A V L D S D G S Y F L Y S K L S V P T S E W Q R G D V F T C S V
M H E A L H N H Y T Q K S I S R S P G K -
SEQ ID NO: 28
Heavy Chain Construct 4 (nucleotide) containing heavy chain sequence (Antibody 2)
linked and in-frame with second heavy chain sequence (Antibody 1). Bold,
underlined, and italicized DNA sequences are restriction sites used for subcloning
to build the constructs (nucleotide) [symbol]
gacgctcaccatggagactgggctgcgctggcttctcctggtcgctgtgctcaaaggtgtccagtgtcagtcgctgg
aggagtccgggggtcgcctggtctcgcctgggacacccctgacactcacctgcaccgcctctggattctccctcagtagcaat
gcaatgggctgggtccgccaggctccaggggaggggctggaatacatcggaatcattaggcctggtggtagcacatactacgc
gagctgggcgaaaggccgattcaccatctccaaaacctcgtcgaccacggtggatctgaaaatgaccagtctgacaaccgagg
acacggccacctatttctgtgccagagcctgggacatctggggcccaggcaccctggtcaccgtctccttagggcaacctaag
gctccatcagtcttcccactggccccctgctgcggggacacacccagctccacggtgaccctgggctgcctggtcaaaggcta
cctcccggagccagtgaccgtgacctggaactcgggcaccctcaccaatggggtacgcaccttcccgtccgtccggcagtcct
caggcctctactcgctgagcagcgtggtgagcgtgacctcaagcagccagcccgtcacctgcaacgtggcccacccagccacc
aacaccaaagtggacaagaccgttgcgccctcgacatgcagcaagcccacgtgcccaccccctgaactcctgg [symbol] ct
caaccccaaccacaaccgcaacctcagccccagccacagccgcagccgcagccacaaccccagcctcaaccacaaccccagcc
tcaacctcag [symbol] cagtcggtggaggagtccgggggtcgcctggtcacgcctgggacacccctgacactcacctgcacag
tctctggattctccctcaataccaatgcaataaggtgggtccgccaggctccagggaaggggctggaatggatcggagccatt
gatgctagtggtaacacatactacgcgagctgggcgaaaggccgattcaccatctccaaaacctcgaccacggtggatctgaa
aatcaccagtccgacaaccgaggacacggccacctatttctgtaacagatttaatactgccaacatctggggcccaggcaccc
tggtcaccgtctcctcagggcaacctaaggctccatcagtcttcccactggccccctgctgcggggacacacccagctccacg
gtgaccctgggctgcctggtcaaaggctacctcccggagccagtgaccgtgacctggaactcgggcaccctcaccaatggggt
acgcaccttcccgtccgtccggcagtcctcaggcctctactcgctgagcagcgtggtgagcgtgacctcaagcagccagcccg
tcacctgcaacgtggcccacccagccaccaacaccaaagtggacaagaccgttgcgccctcgacatgcagcaagcccacgtgc
ccaccccctgaactcctggggggaccgtctgtcttcatcttccccccaaaacccaaggacaccctcatgatctcacgcacccc
cgaggtcacatgcgtggtggtggacgtgagccaggatgaccccgaggtgcagttcacatggtacataaacaacgagcaggtgc
gcaccgcccggccgccgctacgggagcagcagttcaacagcacgatccgcgtggtcagcaccctccccatcgcgcaccaggac
tggctgaggggcaaggagttcaagtgcaaagtccacaacaaggcactcccggcccccatcgagaaaaccatctccaaagccag
agggcagcccctggagccgaaggtctacaccatgggccctccccgggaggagctgagcagcaggtcggtcagcctggcctgca
tgatcaacggatctacccttccgacatctcggtggagtgggagaagaacgggaaggcagaggacaactacaagaccacgccgg
ccgtgctggacagcgacggctcctacttcctctacagcaagctctcagtgcccacgagtgagtggcagcggggcgacgtatca
cctgctccgtgatgcacgaggccttgcacaaccactacacgcagaagtccatctcccgctctccgggtaaatga [symbol]
SEQ ID NO: 29
Heavy Chain Construct 4 (amino acid) containing heavy chain sequence (Antibody 2)
linked and in-frame with second heavy chain sequence (Antibody 1). Italicized amino
acid sequence is signal peptide. Underlined amino acid sequences are two variable
domains in tandem of Antibody 2 and Antibody 1, respectively. Bold amino acid
sequence is PQ Linker.
M E T G L R W L L L V A V L K G V Q C Q S L E E S G G R L V S P G T P L T L T C T A
S G F S L S S N A M G W V R Q A P G E G L E Y I G I I R P G G S T Y Y A S W A K G R
F T I S K T S S T T V D L K M T S L T T E D T A T Y F C A R A W D I W G P G T L V T
V S L G Q P K A P S V F P L A P C C G D T P S S T V T L G C L V K G Y L P E P V T V
T W N S G T L T N G V R T F P S V R Q S S G L Y S L S S V V S V T S S S Q P V T C N
V A H P A T N T K V D K T V A P S T C S K P T C P P P E L L G G R P Q P Q P Q P Q P
Q P Q P Q P Q P Q P Q P Q P Q P Q P Q P Q P Q S R Q S V E E S G G R L V T P G T P L
T L T C T V S G F S L N T N A I R W V R Q A P G K G L E W I G A I D A S G N T Y Y A
S W A K G R F T I S K T S T T V D L K I T S P T T E D T A T Y F C N R F N T A N I W
G P G T L V T V S S G Q P K A P S V F P L A P C C G D T P S S T V T L G C L V K G Y
L P E P V T V T W N S G T L T N G V R T F P S V R Q S S G L Y S L S S V V S V T S S
S Q P V T C N V A H P A T N T K V D K T V A P S T C S K P T C P P P E L L G G P S V
F I F P P K P K D T L M I S R T P E V T C V V V D V S Q D D P E V Q F T W Y I N N E
Q V R T A R P P L R E Q Q F N S T I R V V S T L P I A H Q D W L R G K E F K C K V H
N K A L P A P I E K T I S K A R G Q P L E P K V Y T M G P P R E E L S S R S V S L A
C M I N G F Y P S D I S V E W E K N G K A E D N Y K T T P A V L D S D G S Y F L Y S
K L S V P T S E W Q R G D V F T C S V M H E A L H N H Y T Q K S I S R S P G K -
SEQ ID NO: 30
Heavy Chain Construct 4 (amino acid) containing heavy chain sequence (Antibody 2)
linked and in-frame with second heavy chain sequence (Antibody 1)-without signal
peptide. Underlined amino acid sequences are two variable domains in tandem of
Antibody 2 and Antibody 1, respectively. Bold amino acid sequence is PQ Linker.
Q S L E E S G G R L V S P G T P L T L T C T A S G F S L S S N A M G W V R Q A P G E
G L E Y I G I I R P G G S T Y Y A S W A K G R F T I S K T S S T T V D L K M T S L T
T E D T A T Y F C A R A W D I W G P G T L V T V S L G Q P K A P S V F P L A P C C G
D T P S S T V T L G C L V K G Y L P E P V T V T W N S G T L T N G V R T F P S V R Q
S S G L Y S L S S V V S V T S S S Q P V T C N V A H P A T N T K V D K T V A P S T C
S K P T C P P P E L L G G R P Q P Q P Q P Q P Q P Q P Q P Q P Q P Q P Q P Q P Q P Q
P Q P Q S R Q S V E E S G G R L V T P G T P L T L T C T V S G F S L N T N A I R W V
R Q A P G K G L E W I G A I D A S G N T Y Y A S W A K G R F T I S K T S T T V D L K
I T S P T T E D T A T Y F C N R F N T A N I W G P G T L V T V S S G Q P K A P S V F
P L A P C C G D T P S S T V T L G C L V K G Y L P E P V T V T W N S G T L T N G V R
T F P S V R Q S S G L Y S L S S V V S V T S S S Q P V T C N V A H P A T N T K V D K
T V A P S T C S K P T C P P P E L L G G P S V F I F P P K P K D T L M I S R T P E V
T C V V V D V S Q D D P E V Q F T W Y I N N E Q V R T A R P P L R E Q Q F N S T I R
V V S T L P I A H Q D W L R G K E F K C K V H N K A L P A P I E K T I S K A R G Q P
L E P K V Y T M G P P R E E L S S R S V S L A C M I N G F Y P S D I S V E W E K N G
K A E D N Y K T T P A V L D S D G S Y F L Y S K L S V P T S E W Q R G D V F T C S V
M H E A L H N H Y T Q K S I S R S P G K -
SEQ ID NO: 31
Heavy Chain Construct 5 (nucleotide) containing heavy chain sequence (Antibody 3)
linked and in-frame with second heavy chain sequence (Antibody 3). Bold,
underlined, and italicized DNA sequences are restriction sites used for subcloning
to build the constructs (nucleotide) [symbol]
aggactgagcacagacgactcaccatggagctggggctgagctgggtggtcctggctgctctactacaaggtgtcca
ggctgaggtgcagctggtggagtctgggggaggcttggtgcagcctggggggtctctgaggctctcctgtgtagcctctggaa
acatgttcggcattaacacgatgggctggttccgcgcggctccagggaaagagcgtcagtttgtagcagctgataatttcagt
ggtggcaggaagaatattgacttctccgtgaggggtcgattcaccatgtcaagagacgccgccagcaacacgatatatctaca
aatgaacagcctgaaagttgaggacacggccgtttattactgcgcagcgggtgcgaatacgatgatgattgtggcggacaatt
acgaatactggggtcaggggacccaggtcaccgtctcctcagaacccaagacaccaaaaa [symbol] [symbol] ctcaaccccaaccac
aaccgcaacctcagccccagccacagccgcagccgcagccacaaccccagcctcaaccacaaccccagcctcaacctcag [symbol]
gctcaggtgcagctggtggagtctgggggaggcttggtgcagcctggggggtctctgaggctctcctgtgtagcctc
tggaaacatgttcggcattaacacgatgggctggttccgcgcggctccagggaaagagcgtcagtttgtagcagctgataatt
tcagtggtggcaggaagaatattgacttctccgtgaggggtcgattcaccatgtcaagagacgccgccagcaacacgatatat
ctacaaatgaacagcctgaaagttgaggacacggccgtttattactgcgcagcgggtgcgaatacgatgatgattgtggcgga
caattacgaatactggggtcaggggacccaggtcaccgtctcctcagaacccaagacaccaaaa [symbol] ccacagcctcagc
cgcagccccagccacaaccacaacccaatcctacaacagaatccaagtgtcccaaatgtccagcccctgagctcctgggaggg
ccctcagtcttcatcttccccccgaaacccaaggacgtcctctccatttctgggaggcccgaggtcacgtgcgttgtggtaga
cgtgggccaggaagaccccgaggtcagtttcaactggtacattgatggcgctgaggtgcgaacggccaacacgaggccaaaag
aggaacagttcaacagcacgtaccgcgtggtcagcgtcctgcccatccagcaccaggactggctgacggggaaggaattcaag
tgcaaggtcaacaacaaagctctcccggcccccatcgagaagaccatctccaaggccaaagggcagacccgggagccgcaggt
gtacgccctggccccacaccgggaagagctggccaaggacaccgtgagcgtaacctgcctggtcaaaggatctacccacctga
tatcaacgttgagtggcagaggaacggtcagccggagtcagagggcacctacgccaccacgccaccccagctggacaacgacg
ggacctacttcctctacagcaagctctcggtgggaaagaacacgtggcagcggggagaaaccttcacctgtgtggtgatgcat
gaggccctg [symbol] agcaccggtcaccatcaccaccatcaccaccacgggtga
SEQ ID NO: 32
Heavy Chain Construct 5 (amino acid) containing heavy chain sequence (Antibody 3)
linked and in-frame with second heavy chain sequence (Antibody 3). Italicized
amino acid sequence is signal peptide. Underlined amino acid sequences are two
variable domains in tandem of Antibody 3. Bold amino acid sequence is PQ Linker.
M E L G L S W V V L A A L L Q G V Q A E V Q L V E S G G G L V Q P G G S L R L S C V
A S G N M F G I N T M G W F R A A P G K E R Q F V A A D N F S G G R K N I D F S V R
G R F T M S R D A A S N T I Y L Q M N S L K V E D T A V Y Y C A A G A N T M M I V A
D N Y E Y W G Q G T Q V T V S S E P K T P K S G R P Q P Q P Q P Q P Q P Q P Q P Q P
Q P Q P Q P Q P Q P Q P Q P Q S R A Q V Q L V E S G G G L V Q P G G S L R L S C V A
S G N M F G I N T M G W F R A A P G K E R Q F V A A D N F S G G R K N I D F S V R G
R F T M S R D A A S N T I Y L Q M N S L K V E D T A V Y Y C A A G A N T M M I V A D
N Y E Y W G Q G T Q V T V S S E P K T P K K L P Q P Q P Q P Q P Q P Q P N P T T E S
K C P K C P A P E L L G G P S V F I F P P K P K D V L S I S G R P E V T C V V V D V
G Q E D P E V S F N W Y I D G A E V R T A N T R P K E E Q F N S T Y R V V S V L P I
Q H Q D W L T G K E F K C K V N N K A L P A P I E K T I S K A K G Q T R E P Q V Y A
L A P H R E E L A K D T V S V T C L V K G F Y P P D I N V E W Q R N G Q P E S E G T
Y A T T P P Q L D N D G T Y F L Y S K L S V G K N T W Q R G E T F T C V V M H E A L
G S S T G H H H H H H H H G -
SEQ ID NO: 33
Heavy Chain Construct 5 (amino acid) containing heavy chain sequence (Antibody 3)
linked and in-frame with second heavy chain sequence (Antibody 3)-without signal
peptide. Underlined amino acid sequences are two variable domains in tandem of
Antibody 3. Bold amino acid sequence is PQ Linker.
V Q L V E S G G G L V Q P G G S L R L S C V A S G N M F G I N T M G W F R A A P G K
E R Q F V A A D N F S G G R K N I D F S V R G R F T M S R D A A S N T I Y L Q M N S
L K V E D T A V Y Y C A A G A N T M M I V A D N Y E Y W G Q G T Q V T V S S E P K T
P K S G R P Q P Q P Q P Q P Q P Q P Q P Q P Q P Q P Q P Q P Q P Q P Q P Q S R A Q V
Q L V E S G G G L V Q P G G S L R L S C V A S G N M F G I N T M G W F R A A P G K E
R Q F V A A D N F S G G R K N I D F S V R G R F T M S R D A A S N T I Y L Q M N S L
K V E D T A V Y Y C A A G A N T M M I V A D N Y E Y W G Q G T Q V T V S S E P K T P
K K L P Q P Q P Q P Q P Q P Q P N P T T E S K C P K C P A P E L L G G P S V F I F P
P K P K D V L S I S G R P E V T C V V V D V G Q E D P E V S F N W Y I D G A E V R T
A N T R P K E E Q F N S T Y R V V S V L P I Q H Q D W L T G K E F K C K V N N K A L
P A P I E K T I S K A K G Q T R E P Q V Y A L A P H R E E L A K D T V S V T C L V K
G F Y P P D I N V E W Q R N G Q P E S E G T Y A T T P P Q L D N D G T Y F L Y S K L
S V G K N T W Q R G E T F T C V V M H E A L G S S T G H H H H H H H H G -
SEQ ID NO: 34
Heavy Chain Variable Region of Antibody 1
Q S V E E S G G R L V T P G T P L T L T C T V S G F S L N T N A I R W V R Q A P G K
G L E W I G A I D A S G N T Y Y A S W A K G R F T I S K T S T T V D L I T S P T T E
D T A T Y F C N R F N T A N I W G P G
SEQ ID NO: 35
Heavy Chain Variable Region of Antibody 2
Q S L E E S G G R L V S P G T P L T L T C T A S G F S L S S N A M G W V R Q A P G E
G L E Y I G I I R P G G S T Y Y A S W A K G R F T I S K T S S T T V D L K M T S L T
T E D T A T Y F C A R A W D I W G P G
SEQ ID NO: 36
Heavy Chain Variable Region of Antibody 3
V Q L V E S G G G L V Q P G G S L R L S C V A S G N M F G I N T M G W F R A A P G K
E R Q F V A A D N F S G G R K N I D F S V R G R F T M S R D A A S N T I Y L Q M N S
L K V E D T A V Y Y C A A G A N T M M I V A D N Y E Y W G Q G
SEQ ID NO: 37
Light Chain Construct 1-full length Antibody 1 (Rabbit) (nucleotide)
atggacacgagggcccccactcagctgctggggctcctgctgctctggctcccaggtgccacatttgcgcaagtgctgaccca
gactgcatcgcccgtgtctgcagctgtgggaggcacagtcaccatcagttgccagtccagtcagagtgttyataataccaata
acttatcctggtatcagcagaaaccarggcagcctcccaagcgcctgatctattctgcatccactctggaatctggggtccca
tcgcggttcaaaggcagtggatctgggacacagttcactctcaccatcagcgacctggagtgtgacgatgctgccacttacta
ctgtgcaggcggttatagtgatattaatgctttcggcggagggaccgaggtggtcgtcaaaggtgatccagttgcacctactg
tcctcatatcccaccatctgctgatcttgtggcaactggaacagtcaccatcgtgtgtgtggcgaataaatactttcccgatg
tcaccgtcacctgggaggtggatggcaccacccaaacaactggcatcgagaacagtaaaacaccgcagaattctgcagattgt
acctacaacctcagcagcactctgacactgaccagcacacagtacaacagccacaaagagtacacctgcaaggtgacccaggg
cacgacctcagtcgtccagagcttcaataggggtgactgttag
SEQ ID NO: 38
Light Chain Construct 1-full length Antibody 1 (Rabbit) (amino acid)
M D T R A P T Q L L G L L L L W L P G A T F A Q V L T Q T A S P V S A A V G G T V T
I S C Q S S Q S V X N T N N L S W Y Q Q K P X Q P P K R L I Y S A S T L E S G V P S
R F K G S G S G T Q F T L T I S D L E C D D A A T Y Y C A G G Y S D I N A F G G G T
E V V V K G D P V A P T V L I F P P S A D L V A T G T V T I V C V A N K Y F P D V T
V T W E V D G T T Q T T G I E N S K T P Q N S A D C T Y N L S S T L T L T S T Q Y N
S H K E Y T C K V T Q G T T S V V Q S F N R G D C -
S E Q SEQ ID NO: 39
Light Chain Construct 2-full length Antibody 2 (Rabbit) (nucleotide)
atggacacgagggcccccactcagctgctgggactcctgctgctctggctcccaggtgccagatgtgcattcgaattgaccca
gactccatcctccgtggaggcagctgtgggaggcacagtcaccatcaggtgccaggccagtcagagcattagcaaccaactat
cctggtatcagcagaaaccagggcagcctcccaagcgcctgatctacagggcatccactctggcatctggggtcccagcgcgt
ttcaaaggcagtggatctggtacagcgttcactctcaccatcagcgacctggagtgtgccgatggtgccacttactactgtca
aagtttttattatactgatgttattgattataatgctttcggcggagggaccgaggtggtggtcacaggtgatccagttgcac
ctactgtcctcatcttcccaccagctgctgatcaggtggcaactggaacagtcaccatcgtgtgtgtggcgaataaatacttt
cccgatgtcaccgtcacctgggaggtggatggcaccacccaaacaactggcatcgagaacagtaaaacaccgcagaattctgc
agattgtacctacaacctcagcagcactctgacactgaccagcacacagtacaacagccacaaagagtacacctgcaaggtga
cccagggcacgacctcagtcgtccagagcttcaataggggtgactgttag
SEQ ID NO: 40
Light Chain Construct 2-full length Antibody 2 (Rabbit) (amino acid)
M D T R A P T Q L L G L L L L W L P G A R C A F E L T Q T P S S V E A A V G G T V T
I R C Q A S Q S I S N Q L S W Y Q Q K P G Q P P K R L I Y R A S T L A S G V P A R F
K G S G S G T A F T L T I S D L E C A D G A T Y Y C Q S F Y Y T D V I D Y N A F G G
G T E V V V T G D P V A P T V L I F P P A A D Q V A T G T V T I V C V A N K Y F P D
V T V T W E V D G T T Q T T G I E N S K T P Q N S A D C T Y N L S S T L T L T S T Q
Y N S H K E Y T C K V T Q G T T S V V Q S F N R G D C-

1. A monovalent heavy chain binding protein comprising, in amino to carboxyl terminal order, (a) a first heavy chain variable domain and all or a portion of a CH1 domain linked to (b) a second heavy chain variable domain and all or a portion of a constant region, wherein the first variable domain and CH1 domain are linked to the second variable domain by a linker comprising the amino acid sequence set forth in SEQ ID NO:10.

2. A monovalent heavy chain binding protein comprising, in amino to carboxyl terminal order, (a) a first heavy chain variable domain and all or a portion of a CH1 domain linked to (b) a second heavy chain variable domain and all or a portion of a constant region, wherein the monovalent heavy chain binding protein further comprises a tag linked to the carboxyl terminus of the CH3 domain.

3. The monovalent heavy chain binding protein of claim 1, further comprising a tag.

4. The monovalent heavy chain binding protein of claim 1, wherein the first and second variable domains bind to the same epitope and/or comprise identical amino acid sequences.

5. (canceled)

6. The monovalent heavy chain binding protein of claim 1, wherein the first and second variable domains bind to two different epitopes and/or comprise different amino acid sequences.

7.-9. (canceled)

10. The monovalent heavy chain binding protein of claim 3, wherein the tag is selected from the group consisting of a polyhistidine, CBP, FLAG, GST, Hemaglutinin antigen (HA), HBH, MBP, c-myc, polyhistidine S-tag, SUMO, TAP, TRX, and V5 tag.

11. (canceled)

12. The monovalent heavy chain binding protein of claim 1, wherein the first and second heavy chain variable domains are each paired with a light chain.

13. The monovalent heavy chain binding protein of claim 2, wherein the first and second heavy chain variable domains are each paired with a light chain.

14. A divalent heavy chain binding protein comprising two monovalent heavy chain binding proteins according to claim 1.

15. A divalent heavy chain binding protein comprising two monovalent heavy chain binding proteins according to claim 2.

16. A divalent heavy chain binding protein comprising two monovalent heavy chain binding proteins according to claim 12.

17. A divalent heavy chain binding protein comprising two monovalent heavy chain binding proteins according to claim 13.

18. The monovalent heavy chain binding protein of claim 1, wherein:
(a) the variable domains are derived from a rabbit, mouse, rat, human, goat, chicken, shark, llama, or other camelid species;
(b) the variable domains are derived from a monoclonal antibody;
(c) the binding affinity of the binding protein is synergistically increased compared to the binding affinity of the same binding protein without the first variable domain and CH1 domain; and/or
(d) wherein the constant region is an IgG1, IgG2, IgG3, IgG4, IgM, IgA1, IgA2, IgAsec, IgD, or IgE isotype.

19.-21. (canceled)

22. A composition comprising the monovalent binding protein of claim 1 and a carrier.

23. A kit comprising:
a) the monovalent heavy chain binding protein of claim 1; and
b) instructions for use.

24. A method of detecting the presence or absence of a target molecule of interest in a biological sample comprising (A) contacting a sample with the monovalent heavy chain binding protein of claim 1, wherein the binding protein specifically binds the target molecule of interest, and (B) detecting the presence or absence of at least one complex comprising the binding protein and target molecule of interest.

25. A method of diagnosing a patient as having a disease characterized by a target molecule of interest, comprising (A) contacting a sample obtained from the patient with the monovalent binding protein of claim 1, wherein the binding protein binds the target molecule of interest, and (B) diagnosing the patient as having the disease based on detection of at least one complex comprising the binding protein and target molecule of interest.

26. A method of treating a patient having a disease by administering an effective amount of the monovalent binding protein of claim 1, thereby treating the disease.

27. (canceled)

28. The method of claim 26, wherein the disease is:
(a) a cancer selected from the group consisting of melanoma (e.g., metastatic malignant melanoma), renal cancer (e.g. clear cell carcinoma), prostate cancer (e.g. hormone refractory prostate adenocarcinoma), pancreatic adenocarcinoma, breast cancer, colon cancer, lung cancer (e.g. non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies;
(b) an inflammatory or autoimmune disorder is selected from the group consisting of Crohn's disease, ulcerative colitis, inflammatory bowel disease, inflammatory fibrosis, scleroderma, lung fibrosis, and cirrhosis, rheumatoid arthritis (RA), osteoarthritis, osteoporosis, asthma (including allergic asthma), allergies, chronic obstructive pulmonary disease (COPD), multiple sclerosis, psoriasis, uveitis, graft versus host disease (GVHD), juvenile early-onset Type I diabetes, transplant rejection, SLE, and Sjögren's syndrome; or
(c) an infectious disease selected from the group consisting of human immunodeficiency viruses, hepatitis viruses class A, B and C, Eppstein Barr virus, human cytomegalovirus, human papilloma viruses, and herpes viruses.

29.-38. (canceled)

39. An isolated nucleic acid encoding the monovalent or divalent heavy chain binding protein of claim 1.

40. An expression vector comprising the isolated nucleic acid of claim 39.

41. A host cell comprising the expression vector of claim 40.

42. A method of producing a monovalent binding protein comprising:
a. culturing the host cell of claim 41 in culture medium under conditions wherein the nucleic acid sequence is expressed, thereby producing the binding protein; and
b. recovering the binding protein from the host cell or culture medium.

43.-45. (canceled)


SEQUENCE LISTING (40 sequences)

SEQ ID NO: 1 - DNA, 57 bp, Oryctolagus cuniculus
  CDS (1)..(57)
atggagactgggctgcgctggcttctcctggtcgctgtgctcaaaggtgtccagtgt

SEQ ID NO: 2 - protein, 19 aa, Oryctolagus cuniculus
METGLRWLLLVAVLKGVQC

SEQ ID NO: 3 - DNA, 60 bp, Lama glama
  CDS (1)..(60)
atggagctggggctgagctgggtggtcctggctgctctactacaaggtgtccaggctgag

SEQ ID NO: 4 - protein, 20 aa, Lama glama
MELGLSWVVLAALLQGVQAE

SEQ ID NO: 5 - DNA, 693 bp, Oryctolagus cuniculus
  CDS (1)..(693)
cagtcgctggaggagtccgggggtcgcctggtcacgcctgggacacccctgacactcacctgcacagtctctggaatcgacctcacttcctactacatgacctgggtccgccagggtccagggaaggggctggaatggatcggcatcattagtactgatggtagcgcatcctacgcgaactgggcgaaaggccgattcaccatctccaaaacctcgaccacggtggatctgaaaatcaccagtccgacaaccgacgacacggccacctatttttgtgccagagcttacgatacttatggttcgggttatgatggtgtctttggcatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctg

SEQ ID NO: 6 - protein, 231 aa, Oryctolagus cuniculus
QSLEESGGRLVTPGTPLTLTCTVSGIDLTSYYMTWVRQGPGKGLEWIGIISTDGSASYANWAKGRFTISKTSTTVDLKITSPTTDDTATYFCARAYDTYGSGYDGVFGIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELL

SEQ ID NO: 7 - DNA, 366 bp, Lama glama
  CDS (1)..(366)
gtgcagctggtggagtctgggggaggcttggtgcggcctggggggtctctgagactcatctgtgaagcctttggaagcatcttcagcgtgaataccatgggctggtaccgccagaccccggggaagcagcgggagttggtcgcaattatgactagtgaaggtacgacaaattatgcagatttcgtgaagggccgattcaccatctccagagacaacgccaaggacacagtgtatctacaaatgaacagcctgaattctgaggacacggccgtctattattgtctcttgaaattgccatttacccttaaggagtattggggccaggggacccaggtcaccgtctccgcagaacccaagacaccaaaa

SEQ ID NO: 8 - protein, 122 aa, Lama glama
VQLVESGGGLVRPGGSLRLICEAFGSIFSVNTMGWYRQTPGKQRELVAIMTSEGTTNYADFVKGRFTISRDNAKDTVYLQMNSLNSEDTAVYYCLLKLPFTLKEYWGQGTQVTVSAEPKTPK

SEQ ID NO: 9 - DNA, 96 bp, Artificial Sequence
  Description of Artificial Sequence Synthetic oligonucleotide
  CDS (1)..(96)
cctcaaccccaaccacaaccgcaacctcagccccagccacagccgcagccgcagccacaaccccagcctcaaccacaaccccagcctcaacctcag

SEQ ID NO: 10 - protein, 32 aa, Artificial Sequence
  Description of Artificial Sequence Synthetic polypeptide
PQPQPQPQPQPQPQPQPQPQPQPQPQPQPQPQ

SEQ ID NO: 11 - DNA, 1332 bp, Oryctolagus cuniculus
  CDS (1)..(1332)
cagtcgctggaggagtccgggggtcgcctggtcacgcctgggacacccctgacactcacctgcacagtctctggaatcgacctcacttcctactacatgacctgggtccgccagggtccagggaaggggctggaatggatcggcatcattagtactgatggtagcgcatcctacgcgaactgggcgaaaggccgattcaccatctccaaaacctcgaccacggtggatctgaaaatcaccagtccgacaaccgacgacacggccacctatttttgtgccagagcttacgatacttatggttcgggttatgatggtgtctttggcatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 12 - protein, 443 aa, Oryctolagus cuniculus
QSLEESGGRLVTPGTPLTLTCTVSGIDLTSYYMTWVRQGPGKGLEWIGIISTDGSASYANWAKGRFTISKTSTTVDLKITSPTTDDTATYFCARAYDTYGSGYDGVFGIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 13 - DNA, 1086 bp, Lama glama
  CDS (1)..(1086)
gctcaggtgcagctggtggagtctgggggaggcttggtgcagcctggggggtctctgaggctctcctgtgtagcctctggaaacatgttcggcattaacacgatgggctggttccgcgcggctccagggaaagagcgtcagtttgtagcagctgataatttcagtggtggcaggaagaatattgacttctccgtgaggggtcgattcaccatgtcaagagacgccgccagcaacacgatatatctacaaatgaacagcctgaaagttgaggacacggccgtttattactgcgcagcgggtgcgaatacgatgatgattgtggcggacaattacgaatactggggtcaggggacccaggtcaccgtctcctcagaacccaagacaccaaaaaagcttccacagcctcagccgcagccccagccacaaccacaacccaatcctacaacagaatccaagtgtcccaaatgtccagcccctgagctcctgggagggccctcagtcttcatcttccccccgaaacccaaggacgtcctctccatttctgggaggcccgaggtcacgtgcgttgtggtagacgtgggccaggaagaccccgaggtcagtttcaactggtacattgatggcgctgaggtgcgaacggccaacacgaggccaaaagaggaacagttcaacagcacgtaccgcgtggtcagcgtcctgcccatccagcaccaggactggctgacggggaaggaattcaagtgcaaggtcaacaacaaagctctcccggcccccatcgagaagaccatctccaaggccaaagggcagacccgggagccgcaggtgtacgccctggccccacaccgggaagagctggccaaggacaccgtgagcgtaacctgcctggtcaaaggcttctacccacctgatatcaacgttgagtggcagaggaacggtcagccggagtcagagggcacctacgccaccacgccaccccagctggacaacgacgggacctacttcctctacagcaagctctcggtgggaaagaacacgtggcagcggggagaaaccttcacctgtgtggtgatgcatgaggccctgtga

SEQ ID NO: 14 - protein, 361 aa, Lama glama
AQVQLVESGGGLVQPGGSLRLSCVASGNMFGINTMGWFRAAPGKERQFVAADNFSGGRKNIDFSVRGRFTMSRDAASNTIYLQMNSLKVEDTAVYYCAAGANTMMIVADNYEYWGQGTQVTVSSEPKTPKKLPQPQPQPQPQPQPNPTTESKCPKCPAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYALAPHREELAKDTVSVTCLVKGFYPPDINVEWQRNGQPESEGTYATTPPQLDNDGTYFLYSKLSVGKNTWQRGETFTCVVMHEAL

SEQ ID NO: 15 - DNA, 1194 bp, Artificial Sequence
  Description of Artificial Sequence Synthetic polynucleotide
  CDS (1)..(1194)
gctcaggtgcagctggtggagtctgggggaggcttggtgcagcctggggggtctctgaggctctcctgtgtagcctctggaaacatgttcggcattaacacgatgggctggttccgcgcggctccagggaaagagcgtcagtttgtagcagctgataatttcagtggtggcaggaagaatattgacttctccgtgaggggtcgattcaccatgtcaagagacgccgccagcaacacgatatatctacaaatgaacagcctgaaagttgaggacacggccgtttattactgcgcagcgggtgcgaatacgatgatgattgtggcggacaattacgaatactggggtcaggggacccaggtcaccgtctcctcagaacccaagacaccaaaaaagcttccacagcctcagccgcagccccagccacaaccacaacccaatcctacaacagaatccaagtgtcccaaatgtccagcccctgagctcctgggagggccctcagtcttcatcttccccccgaaacccaaggacgtcctctccatttctgggaggcccgaggtcacgtgcgttgtggtagacgtgggccaggaagaccccgaggtcagtttcaactggtacattgatggcgctgaggtgcgaacggccaacacgaggccaaaagaggaacagttcaacagcacgtaccgcgtggtcagcgtcctgcccatccagcaccaggactggctgacggggaaggaattcaagtgcaaggtcaacaacaaagctctcccggcccccatcgagaagaccatctccaaggccaaagggcagacccgggagccgcaggtgtacgccctggccccacaccgggaagagctggccaaggacaccgtgagcgtaacctgcctggtcaaaggcttctacccacctgatatcaacgttgagtggcagaggaacggtcagccggagtcagagggcacctacgccaccacgccaccccagctggacaacgacgggacctacttcctctacagcaagctctcggtgggaaagaacacgtggcagcggggagaaaccttcacctgtgtggtgatgcatgaggccctggttccctctagagggcccttcgaaggtaagcctatccctaaccctctcctcggtctcgattctacgcgtaccggtcatcatcaccatcaccatcaccatggaggacagtga

SEQ ID NO: 16 - protein, 397 aa, Artificial Sequence
  Description of Artificial Sequence Synthetic polypeptide
AQVQLVESGGGLVQPGGSLRLSCVASGNMFGINTMGWFRAAPGKERQFVAADNFSGGRKNIDFSVRGRFTMSRDAASNTIYLQMNSLKVEDTAVYYCAAGANTMMIVADNYEYWGQGTQVTVSSEPKTPKKLPQPQPQPQPQPQPNPTTESKCPKCPAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYALAPHREELAKDTVSVTCLVKGFYPPDINVEWQRNGQPESEGTYATTPPQLDNDGTYFLYSKLSVGKNTWQRGETFTCVVMHEALVPSRGPFEGKPIPNPLLGLDSTRTGHHHHHHHHGGQ

SEQ ID NO: 17 - DNA, 480 bp, Artificial Sequence
  Description of Artificial Sequence Synthetic polynucleotide
  CDS (1)..(441)
gtgcagctggtggagtctgggggaggcttggtgcagcctggggggtctctgaggctctcctgtgtagcctctggaaacatgttcggcattaacacgatgggctggttccgcgcggctccagggaaagagcgtcagtttgtagcagctgataatttcagtggtggcaggaagaatattgacttctccgtgaggggtcgattcaccatgtcaagagacgccgccagcaacacgatatatctacaaatgaacagcctgaaagttgaggacacggccgtttattactgcgcagcgggtgcgaatacgatgatgattgtggcggacaattacgaatactggggtcaggggacccaggtcaccgtctcctcagaacccaagacaccaaaaaagcttgatatcacgcgtaccggtcatcatcaccatcaccatcaccatggaggacagtgaagatcccccgacctcgacctctggctaataaaggaa

SEQ ID NO: 18 - protein, 147 aa, Artificial Sequence
  Description of Artificial Sequence Synthetic polypeptide
VQLVESGGGLVQPGGSLRLSCVASGNMFGINTMGWFRAAPGKERQFVAADNFSGGRKNIDFSVRGRFTMSRDAASNTIYLQMNSLKVEDTAVYYCAAGANTMMIVADNYEYWGQGTQVTVSSEPKTPKKLDITRTGHHHHHHHHGGQ

SEQ ID NO: 19 - DNA, 2161 bp, Artificial Sequence
  Description of Artificial Sequence Synthetic polynucleotide
  CDS (17)..(2152)
gctagcgacgctcaccatggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcaataccaatgcaataaggtgggtccgccaggctccagggaaggggctggaatggatcggagccattgatgctagtggtaacacatactacgcgagctgggcgaaaggccgattcaccatctccaaaacctcgaccacggtggatctgaaaatcaccagtccgacaaccgaggacacggccacctatttctgtaacagatttaatactgccaacatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctgggcggccgccctcaaccccaaccacaaccgcaacctcagccccagccacagccgcagccgcagccacaaccccagcctcaaccacaaccccagcctcaacctcagtctagacagtcggtggaggagtccgggggtcgcctggtcacgcctgggacacccctgacactcacctgcacagtctctggattctccctcaataccaatgcaataaggtgggtccgccaggctccagggaaggggctggaatggatcggagccattgatgctagtggtaacacatactacgcgagctgggcgaaaggccgattcaccatctccaaaacctcgaccacggtggatctgaaaatcaccagtccgacaaccgaggacacggccacctatttctgtaacagatttaatactgccaacatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatgaggatcc

SEQ ID NO: 20 - protein, 712 aa, Artificial Sequence
  Description of Artificial Sequence Synthetic polypeptide
METGLRWLLLVAVLKGVQCQSVEESGGRLVTPGTPLTLTCTVSGFSLNTNAIRWVRQAPGKGLEWIGAIDASGNTYYASWAKGRFTISKTSTTVDLKITSPTTEDTATYFCNRFNTANIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGRPQPQPQPQPQPQPQPQPQPQPQPQPQPQPQPQSRQSVEESGGRLVTPGTPLTLTCTVSGFSLNTNAIRWVRQAPGKGLEWIGAIDASGNTYYASWAKGRFTISKTSTTVDLKITSPTTEDTATYFCNRFNTANIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 21 - protein, 673 aa, Artificial Sequence
  Description of Artificial Sequence Synthetic polypeptide
QSVEESGGRLVTPGTPLTLTCTVSGFSLNTNAIRWVRQAPGKGLEWIGAIDASGNTYYASWAKGRFTISKTSTTVDLKITSPTTEDTATYFCNRFNTANIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGRPQPQPQPQPQPQPQPQPQPQPQPQPQPQPQPQSRQSVEESGLNTNAIRWVRQAPGKGLEWIGAIDASGNTYYASWAKGRFTISKTSTTVDLKITSPTTEDTATYFCNRFNTANIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 22 - DNA, 2155 bp, Artificial Sequence
  Description of Artificial Sequence Synthetic polynucleotide
  CDS (17)..(2146)
gctagcgacgctcaccatggagactgggctgcgctggcttctcctggtcgctgtgctcaaaggtgtccagtgtcagtcgctggaggagtccgggggtcgcctggtctcgcctgggacacccctgacactcacctgcaccgcctctggattctccctcagtagcaatgcaatgggctgggtccgccaggctccaggggaggggctggaatacatcggaatcattaggcctggtggtagcacatactacgcgagctgggcgaaaggccgattcaccatctccaaaacctcgtcgaccacggtggatctgaaaatgaccagtctgacaaccgaggacacggccacctatttctgtgccagagcctgggacatctggggcccaggcaccctggtcaccgtctccttagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctgggcggccgccctcaaccccaaccacaaccgcaacctcagccccagccacagccgcagccgcagccacaaccccagcctcaaccacaaccccagcctcaacctcagtctagacagtcgctggaggagtccgggggtcgcctggtctcgcctgggacacccctgacactcacctgcaccgcctctggattctccctcagtagcaatgcaatgggctgggtccgccaggctccaggggaggggctggaatacatcggaatcattaggcctggtggtagcacatactacgcgagctgggcgaaaggccgattcaccatctccaaaacctcgtcgaccacggtggatctgaaaatgaccagtctgacaaccgaggacacggccacctatttctgtgccagagcctgggacatctggggcccaggcaccctggtcaccgtctccttagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatgaggatcc

SEQ ID NO: 23 - protein, 710 aa, Artificial Sequence
  Description of Artificial Sequence Synthetic polypeptide
METGLRWLLLVAVLKGVQCQSLEESGGRLVSPGTPLTLTCTASGFSLSSNAMGWVRQAPGEGLEYIGIIRPGGSTYYASWAKGRFTISKTSSTTVDLKMTSLTTEDTATYFCARAWDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGRPQPQPQPQPQPQPQPQPQPQPQPQPQPQPQPQSRQSLEESGGRLVSPGTPLTLTCTASGFSLSSNAMGWVRQAPGEGLEYIGIIRPGGSTYYASWAKGRFTISKTSSTTVDLKMTSLTTEDTATYFCARAWDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 24 - protein, 691 aa, Artificial Sequence
  Description of Artificial Sequence Synthetic polypeptide
QSLEESGGRLVSPGTPLTLTCTASGFSLSSNAMGWVRQAPGEGLEYIGIIRPGGSTYYASWAKGRFTISKTSSTTVDLKMTSLTTEDTATYFCARAWDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGRPQPQPQPQPQPQPQPQPQPQPQPQPQPQPQPQSRQSLEESGGRLVSPGTPLTLTCTASGFSLSSNAMGWVRQAPGEGLEYIGIIRPGGSTYYASWAKGRFTISKTSSTTVDLKMTSLTTEDTATYFCARAWDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 25 - DNA, 2158 bp, Artificial Sequence
  Description of Artificial Sequence Synthetic polynucleotide
  CDS (17)..(2149)
gctagcgacgctcaccatggagactgggctgcgctggcttctcctggtcgctgtgctcaaaggtgtccagtgtcagtcggtggaggagtccgggggtcgcctggtcacgcctgggacacccctgacactcacctgcacagtctctggattctccctcaataccaatgcaataaggtgggtccgccaggctccagggaaggggctggaatggatcggagccattgatgctagtggtaacacatactacgcgagctgggcgaaaggccgattcaccatctccaaaacctcgaccacggtggatctgaaaatcaccagtccgacaaccgaggacacggccacctatttctgtaacagatttaatactgccaacatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccatgtgcccaccccctgaactcctgggcggccgccctcaaccccaaccacaaccgcaacctcagccccagccacagccgcagccgcagccacaaccccagcctcaaccacaaccccagcctcaacctcagtctagacagtcgctggaggagtccgggggtcgcctggtctcgcctgggacacccctgacactcacctgcaccgcctctggattctccctcagtagcaatgcaatgggctgggtccgccaggctccaggggaggggctggaatacatcggaatcattaggcctggtggtagcacatactacgcgagctgggcgaaaggccgattcaccatctccaaaacctcgtcgaccacggtggatctgaaaatgaccagtctgacaaccgaggacacggccacctatttctgtgccagagcctgggacatctggggcccaggcaccctggtcaccgtctccttagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatgaggatcc

SEQ ID NO: 26 - protein, 711 aa, Artificial Sequence
  Description of Artificial Sequence Synthetic polypeptide
METGLRWLLLVAVLKGVQCQSVEESGGRLVTPGTPLTLTCTVSGFSLNTNAIRWVRQAPGKGLEWIGAIDASGNTYYASWAKGRFTISKTSTTVDLKITSPTTEDTATYFCNRFNTANIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGRPQPQPQPQPQPQPQPQPQPQPQPQPQPQPQPQSRQSLEESGGRLVSPGTPLTLTCTASGFSLSSNAMGWVRQAPGEGLEYIGIIRPGGSTYYASWAKGRFTISKTSSTTVDLKMTSLTTEDTATYFCARAWDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 27 - protein, 692 aa, Artificial Sequence
  Description of Artificial Sequence Synthetic polypeptide
QSVEESGGRLVTPGTPLTLTCTVSGFSLNTNAIRWVRQAPGKGLEWIGAIDASGNTYYASWAKGRFTISKTSTTVDLKITSPTTEDTATYFCNRFNTANIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPMCPPPELLGGRPQPQPQPQPQPQPQPQPQPQPQPQPQPQPQPQSRQSLEESGGRLVSPGTPLTLTCTASGFSLSSNAMGWVRQAPGEGLEYIGIIRPGGSTYYASWAKGRFTISKTSSTTVDLKMTSLTTEDTATYFCARAWDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 28 - DNA, 2158 bp, Artificial Sequence
  Description of Artificial Sequence Synthetic polynucleotide
  CDS (17)..(2149)
gctagcgacgctcaccatggagactgggctgcgctggcttctcctggtcgctgtgctcaaaggtgtccagtgtcagtcgctggaggagtccgggggtcgcctggtctcgcctgggacacccctgacactcacctgcaccgcctctggattctccctcagtagcaatgcaatgggctgggtccgccaggctccaggggaggggctggaatacatcggaatcattaggcctggtggtagcacatactacgcgagctgggcgaaaggccgattcaccatctccaaaacctcgtcgaccacggtggatctgaaaatgaccagtctgacaaccgaggacacggccacctatttctgtgccagagcctgggacatctggggcccaggcaccctggtcaccgtctccttagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctgggcggccgccctcaaccccaaccacaaccgcaacctcagccccagccacagccgcagccgcagccacaaccccagcctcaaccacaaccccagcctcaacctcagtctagacagtcggtggaggagtccgggggtcgcctggtcacgcctgggacacccctgacactcacctgcacagtctctggattctccctcaataccaatgcaataaggtgggtccgccaggctccagggaaggggctggaatggatcggagccattgatgctagtggtaacacatactacgcgagctgggcgaaaggccgattcaccatctccaaaacctcgaccacggtggatctgaaaatcaccagtccgacaaccgaggacacggccacctatttctgtaacagatttaatactgccaacatctggggcccaggcaccctggtcaccgtctcctcagggcaacctaaggctccatcagtcttcccactggccccctgctgcggggacacacccagctccacggtgaccctgggctgcctggtcaaaggctacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctggcctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatgaggatcc

SEQ ID NO: 29 - protein, 711 aa, Artificial Sequence
  Description of Artificial Sequence Synthetic polypeptide
METGLRWLLLVAVLKGVQCQSLEESGGRLVSPGTPLTLTCTASGFSLSSNAMGWVRQAPGEGLEYIGIIRPGGSTYYASWAKGRFTISKTSSTTVDLKMTSLTTEDTATYFCARAWDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGRPQPQPQPQPQPQPQPQPQPQPQPQPQPQPQPQSRQSVEESGGRLVTPGTPLTLTCTVSGFSLNTNAIRWVRQAPGKGLEWIGAIDASGNTYYASWAKGRFTISKTSTTVDLKITSPTTEDTATYFCNRFNTANIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 30 - protein, 692 aa, Artificial Sequence
  Description of Artificial Sequence Synthetic polypeptide
QSLEESGGRLVSPGTPLTLTCTASGFSLSSNAMGWVRQAPGEGLEYIGIIRPGGSTYYASWAKGRFTISKTSSTTVDLKMTSLTTEDTATYFCARAWDIWGPGTLVTVSLGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGRPQPQPQPQPQPQPQPQPQPQPQPQPQPQPQPQSRQSVEESGGRLVTPGTPLTLTCTVSGFSLNTNAIRWVRQAPGKGLEWIGAIDASGNTYYASWAKGRFTISKTSTTVDLKITSPTTEDTATYFCNRFNTANIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLACMINGFYPSDISVEWEKNGKAEDNYKTTPAVLDSDGSYFLYSKLSVPTSEWQRGDVFTCSVMHEALHNHYTQKSISRSPGK

SEQ ID NO: 31 - DNA, 1713 bp, Artificial Sequence
  Description of Artificial Sequence Synthetic polynucleotide
  CDS (31)..(1710)
gctagcaggactgagcacagacgactcaccatggagctggggctgagctgggtggtcctggctgctctactacaaggtgtccaggctgaggtgcagctggtggagtctgggggaggcttggtgcagcctggggggtctctgaggctctcctgtgtagcctctggaaacatgttcggcattaacacgatgggctggttccgcgcggctccagggaaagagcgtcagtttgtagcagctgataatttcagtggtggcaggaagaatattgacttctccgtgaggggtcgattcaccatgtcaagagacgccgccagcaacacgatatatctacaaatgaacagcctgaaagttgaggacacggccgtttattactgcgcagcgggtgcgaatacgatgatgattgtggcggacaattacgaatactggggtcaggggacccaggtcaccgtctcctcagaacccaagacaccaaaaagcggccgccctcaaccccaaccacaaccgcaacctcagccccagccacagccgcagccgcagccacaaccccagcctcaaccacaaccccagcctcaacctcagtctagagctcaggtgcagctggtggagtctgggggaggcttggtgcagcctggggggtctctgaggctctcctgtgtagcctctggaaacatgttcggcattaacacgatgggctggttccgcgcggctccagggaaagagcgtcagtttgtagcagctgataatttcagtggtggcaggaagaatattgacttctccgtgaggggtcgattcaccatgtcaagagacgccgccagcaacacgatatatctacaaatgaacagcctgaaagttgaggacacggccgtttattactgcgcagcgggtgcgaatacgatgatgattgtggcggacaattacgaatactggggtcaggggacccaggtcaccgtctcctcagaacccaagacaccaaaaaagcttccacagcctcagccgcagccccagccacaaccacaacccaatcctacaacagaatccaagtgtcccaaatgtccagcccctgagctcctgggagggccctcagtcttcatcttccccccgaaacccaaggacgtcctctccatttctgggaggcccgaggtcacgtgcgttgtggtagacgtgggccaggaagaccccgaggtcagtttcaactggtacattgatggcgctgaggtgcgaacggccaacacgaggccaaaagaggaacagttcaacagcacgtaccgcgtggtcagcgtcctgcccatccagcaccaggactggctgacggggaaggaattcaagtgcaaggtcaacaacaaagctctcccggcccccatcgagaagaccatctccaaggccaaagggcagacccgggagccgcaggtgtacgccctggccccacaccgggaagagctggccaaggacaccgtgagcgtaacctgcctggtcaaaggcttctacccacctgatatcaacgttgagtggcagaggaacggtcagccggagtcagagggcacctacgccaccacgccaccccagctggacaacgacgggacctacttcctctacagcaagctctcggtgggaaagaacacgtggcagcggggagaaaccttcacctgtgtggtgatgcatgaggccctgggatccagcaccggtcaccatcaccaccatcaccaccacgggtga

SEQ ID NO: 32 - protein, 560 aa, Artificial Sequence
  Description of Artificial Sequence Synthetic polypeptide
MELGLSWVVLAALLQGVQAEVQLVESGGGLVQPGGSLRLSCVASGNMFGINTMGWFRAAPGKERQFVAADNFSGGRKNIDFSVRGRFTMSRDAASNTIYLQMNSLKVEDTAVYYCAAGANTMMIVADNYEYWGQGTQVTVSSEPKTPKSGRPQPQPQPQPQPQPQPQPQPQPQPQPQPQPQPQSRAQVQLVESGGGLVQPGGSLRLSCVASGNMFGINTMGWFRAAPGKERQFVAADNFSGGRKNIDFSVRGRFTMSRDAASNTIYLQMNSLKVEDTAVYYCAAGANTMMIVADNYEYWGQGTQVTVSSEPKTPKKLPQPQPQPQPQPQPNPTTESKCPKCPAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYALAPHREELAKDTVSVTCLVKGFYPPDINVEWQRNGQPESEGTYATTPPQLDNDGTYFLYSKLSVGKNTWQRGETFTCVVMHEALGSSTGHHHHHHHHG

SEQ ID NO: 33 - protein, 540 aa, Artificial Sequence
  Description of Artificial Sequence Synthetic polypeptide
VQLVESGGGLVQPGGSLRLSCVASGNMFGINTMGWFRAAPGKERQFVAADNFSGGRKNIDFSVRGRFTMSRDAASNTIYLQMNSLKVEDTAVYYCAAGANTMMIVADNYEYWGQGTQVTVSSEPKTPKSGRPQPQPQPQPQPQPQPQPQPQPQPQPQPQPQPQSRAQVQLVESGGGLVQPGGSLRLSCVASGNMFGINTMGWFRAAPGKERQFVAADNFSGGRKNIDFSVRGRFTMSRDAASNTIYLQMNSLKVEDTAVYYCAAGANTMMIVADNYEYWGQGTQVTVSSEPKTPKKLPQPQPQPQPQPQPNPTTESKCPKCPAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYALAPHREELAKDTVSVTCLVKGFYPPDINVEWQRNGQPESEGTYATTPPQLDNDGTYFLYSKLSVGKNTWQRGETFTCVVMHEALGSSTGHHHHHHHHG

SEQ ID NO: 34 - protein, 103 aa, Artificial Sequence
  Description of Artificial Sequence Synthetic polypeptide
QSVEESGGRLVTPGTPLTLTCTVSGFSLNTNAIRWVRQAPGKGLEWIGAIDASGNTYYASWAKGRFTISKTSTTVDLITSPTTEDTATYFCNRFNTANIWGPG

SEQ ID NO: 35 - protein, 103 aa, Artificial Sequence
  Description of Artificial Sequence Synthetic polypeptide
QSLEESGGRLVSPGTPLTLTCTASGFSLSSNAMGWVRQAPGEGLEYIGIIRPGGSTYYASWAKGRFTISKTSSTTVDLKMTSLTTEDTATYFCARAWDIWGPG

SEQ ID NO: 36 - protein, 115 aa, Artificial Sequence
  Description of Artificial Sequence Synthetic polypeptide
VQLVESGGGLVQPGGSLRLSCVASGNMFGINTMGWFRAAPGKERQFVAADNFSGGRKNIDFSVRGRFTMSRDAASNTIYLQMNSLKVEDTAVYYCAAGANTMMIVADNYEYWGQG

SEQ ID NO: 37 - DNA, 708 bp, Artificial Sequence
  Description of Artificial Sequence Synthetic polynucleotide
  CDS (1)..(705)
atggacacgagggcccccactcagctgctggggctcctgctgctctggctcccaggtgccacatttgcgcaagtgctgacccagactgcatcgcccgtgtctgcagctgtgggaggcacagtcaccatcagttgccagtccagtcagagtgttyataataccaataacttatcctggtatcagcagaaaccarggcagcctcccaagcgcctgatctattctgcatccactctggaatctggggtcccatcgcggttcaaaggcagtggatctgggacacagttcactctcaccatcagcgacctggagtgtgacgatgctgccacttactactgtgcaggcggttatagtgatattaatgctttcggcggagggaccgaggtggtcgtcaaaggtgatccagttgcacctactgtcctcatcttcccaccatctgctgatcttgtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 38 - protein, 235 aa, Artificial Sequence
  Description of Artificial Sequence Synthetic polypeptide
  MOD_RES (52)..(52): Any amino acid
  MOD_RES (65)..(65): Any amino acid
MDTRAPTQLLGLLLLWLPGATFAQVLTQTASPVSAAVGGTVTISCQSSQSVXNTNNLSWYQQKPXQPPKRLIYSASTLESGVPSRFKGSGSGTQFTLTISDLECDDAATYYCAGGYSDINAFGGGTEVVVKGDPVAPTVLIFPPSADLVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 39 - DNA, 714 bp, Artificial Sequence
  Description of Artificial Sequence Synthetic polynucleotide
  CDS (1)..(711)
atggacacgagggcccccactcagctgctgggactcctgctgctctggctcccaggtgccagatgtgcattcgaattgacccagactccatcctccgtggaggcagctgtgggaggcacagtcaccatcaggtgccaggccagtcagagcattagcaaccaactatcctggtatcagcagaaaccagggcagcctcccaagcgcctgatctacagggcatccactctggcatctggggtcccagcgcgtttcaaaggcagtggatctggtacagcgttcactctcaccatcagcgacctggagtgtgccgatggtgccacttactactgtcaaagtttttattatactgatgttattgattataatgctttcggcggagggaccgaggtggtggtcacaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 40 - protein, 237 aa, Artificial Sequence
  Description of Artificial Sequence Synthetic polypeptide
MDTRAPTQLLGLLLLWLPGARCAFELTQTPSSVEAAVGGTVTIRCQASQSISNQLSWYQQKPGQPPKRLIYRASTLASGVPARFKGSGSGTAFTLTISDLECADGATYYCQSFYYTDVIDYNAFGGGTEVVVTGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC